SEQUENCE LISTING (201 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 5 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 9 - protein, 5 aa, synthetic construct
GGGGS

SEQ ID NO: 10 - protein, 10 aa, synthetic construct
GGGGSGGGGS

SEQ ID NO: 11 - protein, 15 aa, synthetic construct
GGGGSGGGGSGGGGS

SEQ ID NO: 12 - protein, 20 aa, synthetic construct
GGGGSGGGGSGGGGSGGGGS

SEQ ID NO: 13 - protein, 25 aa, synthetic construct
GGGGSGGGGSGGGGSGGGGSGGGGS

SEQ ID NO: 14 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 15 - protein, 301 aa, Macaca fascicularis
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 16 - protein, 120 aa, synthetic construct [sequence omitted]

SEQ ID NO: 17 - protein, 5 aa, synthetic construct
SYTMH

SEQ ID NO: 18 - protein, 17 aa, synthetic construct
YINPRSGYTHYNQKLKD

SEQ ID NO: 19 - protein, 11 aa, synthetic construct
SAYYDYDGFAY

SEQ ID NO: 20 - protein, 106 aa, synthetic construct [sequence omitted]

SEQ ID NO: 21 - protein, 10 aa, synthetic construct
SASSSVSYMN

SEQ ID NO: 22 - protein, 7 aa, synthetic construct
DTSKLAS

SEQ ID NO: 23 - protein, 9 aa, synthetic construct
QQWSSNPPT

SEQ ID NO: 24 - protein, 125 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 25 - protein, 5 aa, synthetic construct
TYAMN

SEQ ID NO: 26 - protein, 19 aa, synthetic construct
RIRSKYNNYATYYADSVKD

SEQ ID NO: 27 - protein, 14 aa, synthetic construct
HANFGAGYVSWFAH

SEQ ID NO: 28 - protein, 109 aa, synthetic construct
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 29 - protein, 14 aa, synthetic construct
GSSTGAVTTSNYAN

SEQ ID NO: 30 - protein, 7 aa, synthetic construct
GTDKRAP

SEQ ID NO: 31 - protein, 9 aa, synthetic construct
ALWYSNHWV

SEQ ID NO: 32 - protein, 109 aa, synthetic construct
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 33 - protein, 9 aa, synthetic construct
ALWYSDLWV

SEQ ID NO: 34 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 35 - protein, 5 aa, synthetic construct
DYYMH

SEQ ID NO: 36 - protein, 17 aa, synthetic construct
WIDLENANTIYDAKFQG

SEQ ID NO: 37 - protein, 10 aa, synthetic construct
DAYGRYFYDV

SEQ ID NO: 38 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 39 - protein, 17 aa, synthetic construct
KSSQSLLNARTGKNYLA

SEQ ID NO: 40 - protein, 7 aa, synthetic construct
WASTRES

SEQ ID NO: 41 - protein, 8 aa, synthetic construct
KQSYSRRT

SEQ ID NO: 42 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 43 - protein, 17 aa, synthetic construct
WIDLENANTVYDAKFQG

SEQ ID NO: 44 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 45 - protein, 8 aa, synthetic construct
KQSYFRRT

SEQ ID NO: 46 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSS

SEQ ID NO: 47 - protein, 10 aa, synthetic construct
DAYGQYFYDV

SEQ ID NO: 48 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 49 - protein, 8 aa, synthetic construct
TQSYFRRT

SEQ ID NO: 50 - protein, 125 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 51 - protein, 14 aa, synthetic construct
HGNFGNSYVSWFAY

SEQ ID NO: 52 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 53 - protein, 14 aa, synthetic construct
RSSTGAVTTSNYAN

SEQ ID NO: 54 - protein, 7 aa, synthetic construct
GTNKRAP

SEQ ID NO: 55 - protein, 9 aa, synthetic construct
ALWYSNLWV

SEQ ID NO: 56 - protein, 118 aa, synthetic construct
QVQLQQSGAELVKPGASVKISCKASGYTFSNYWIEWVKQRPGLGLEWIGDILPGSGTTNYNEKFKGKATFTADTSSNTAYMQLSSLTSEDSAVYYCAGYGNYLWFTYWGQGTLVTVSA

SEQ ID NO: 57 - protein, 10 aa, synthetic construct
GYTFSNYWIE

SEQ ID NO: 58 - protein, 17 aa, synthetic construct
DILPGSGTTNYNEKFKG

SEQ ID NO: 59 - protein, 9 aa, synthetic construct
YGNYLWFTY

SEQ ID NO: 60 - protein, 107 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIK

SEQ ID NO: 61 - protein, 11 aa, synthetic construct
HASQNINVWLS

SEQ ID NO: 62 - protein, 7 aa, synthetic construct
KASNLHT

SEQ ID NO: 63 - protein, 9 aa, synthetic construct
QQGQSYPYT

SEQ ID NO: 64 - protein, 122 aa, synthetic construct
QAYLQQSGAEVVRSGASVKMSCKAAGYTFTSYNMHWVKQTPGQGLEWIGYIYPGNGGTNYNQKFKGKATLTADTSSTTAYMQISSLTSEDSAVYFCARGASMITTGAWFAYWGQGTLVTVSA

SEQ ID NO: 65 - protein, 10 aa, synthetic construct
GYTFTSYNMH

SEQ ID NO: 66 - protein, 17 aa, synthetic construct
YIYPGNGGTNYNQKFKG

SEQ ID NO: 67 - protein, 13 aa, synthetic construct
GASMITTGAWFAY

SEQ ID NO: 68 - protein, 111 aa, synthetic construct
NIVLTQSPASLAVSLGQRATISCRASESLDTYGNSFMYWYQQKPGQPPKLLIYLASSLESGVPARFSGSGSRTDFTLTIDPVEADDAATYYCHQNHEDPWTFGGGTKLEIK

SEQ ID NO: 69 - protein, 15 aa, synthetic construct
RASESLDTYGNSFMY

SEQ ID NO: 70 - protein, 7 aa, synthetic construct
LASSLES

SEQ ID NO: 71 - protein, 9 aa, synthetic construct
HQNHEDPWT

SEQ ID NO: 72 - protein, 119 aa, synthetic construct
EVQLQQSGTVLARPGASVKMSCKASGYTFTRYWMHWVKQRPGQDLEWIGGFYPGNSDTRYNQKFKDKAKLTAVTSTSTAYMELSSLTNEDSAVYYCTRGDRHDEGFAYWGQGTLVTVSA

SEQ ID NO: 73 - protein, 10 aa, synthetic construct
GYTFTRYWMH

SEQ ID NO: 74 - protein, 17 aa, synthetic construct
GFYPGNSDTRYNQKFKD

SEQ ID NO: 75 - protein, 10 aa, synthetic construct
GDRHDEGFAY

SEQ ID NO: 76 - protein, 106 aa, synthetic construct
DIQMTQSPASLSASVGETVTITCRASGNIHNYLAWYQQKQGKSPQLLVYNAKTLPDGVPSRFSGSGSGTQYSLKINSLQPEDFGTYYCQNIWTNYTFGGGTKLEIK

SEQ ID NO: 77 - protein, 11 aa, synthetic construct
RASGNIHNYLA

SEQ ID NO: 78 - protein, 7 aa, synthetic construct
NAKTLPD

SEQ ID NO: 79 - protein, 8 aa, synthetic construct
QNIWTNYT

SEQ ID NO: 80 - protein, 123 aa, synthetic construct
QIQLVQSGPELKKPGETVKISCKASGYTFTNYGMNWVKQAPGEDLKWMGWLNTNTGEPTYAEEFKGRFAFSLETSASTAYLQINNLRNEDTATYFCARWYYGSSDNSYAMDYWGQGTSVTVSS

SEQ ID NO: 81 - protein, 10 aa, synthetic construct
GYTFTNYGMN

SEQ ID NO: 82 - protein, 17 aa, synthetic construct
WLNTNTGEPTYAEEFKG

SEQ ID NO: 83 - protein, 14 aa, synthetic construct
WYYGSSDNSYAMDY

SEQ ID NO: 84 - protein, 107 aa, synthetic construct
ETIVTQFPASLSMAIGEKVTIRCITSTDIDDDMNWYQQRPGEPPKLLISEGNTLRPGVPSRFSSSGYGTDFVFTFENMLSEDVADYYCLQSDSLPYTVGGGTRLEIK

SEQ ID NO: 85 - protein, 11 aa, synthetic construct
ITSTDIDDDMN

SEQ ID NO: 86 - protein, 7 aa, synthetic construct
EGNTLRP

SEQ ID NO: 87 - protein, 9 aa, synthetic construct
LQSDSLPYT

SEQ ID NO: 88 - protein, 117 aa, synthetic construct
QVQLQQSGAELVRPGTSVKMSCKAAGYTFTNHWIEWVKQRPGHGLEWIGDIFPGGGYTDYNEKFKGKATLTADTSSSTAYMQLSSLTSEDSAIYYCARGRDLAMDYWGQGTSVTVSS

SEQ ID NO: 89 - protein, 10 aa, synthetic construct
GYTFTNHWIE

SEQ ID NO: 90 - protein, 17 aa, synthetic construct
DIFPGGGYTDYNEKFKG

SEQ ID NO: 91 - protein, 8 aa, synthetic construct
GRDLAMDY

SEQ ID NO: 92 - protein, 107 aa, synthetic construct
DIQMNQSPSSLSASLGDTITITCHASQNINVWLGWYQQKPGNIPKLLIYKASNLHTGVPSRFSGSGSGTGFTLTISSLQPEDIATYYCQQGQSYPYTFGGGTKLEIK

SEQ ID NO: 93 - protein, 11 aa, synthetic construct
HASQNINVWLG

SEQ ID NO: 94 - protein, 7 aa, synthetic construct
KASNLHT

SEQ ID NO: 95 - protein, 9 aa, synthetic construct
QQGQSYPYT

SEQ ID NO: 96 - protein, 117 aa, synthetic construct
EVQLQQSGAEFVRPGASVKLSCTASGFNIKDSLMHWVKQRPEQGLEWIGWIDPEDDKTKYAPKFQDEATLTADTSSNTAYLHLSSLTSEDTAIYYCARRFLRGFAYWGQGTLVTVSA

SEQ ID NO: 97 - protein, 10 aa, synthetic construct
GFNIKDSLMH

SEQ ID NO: 98 - protein, 17 aa, synthetic construct
WIDPEDDKTKYAPKFQD

SEQ ID NO: 99 - protein, 8 aa, synthetic construct
RFLRGFAY

SEQ ID NO: 100 - protein, 113 aa, synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPYTFGGGTKLEIK

SEQ ID NO: 101 - protein, 17 aa, synthetic construct
KSSQSLLNSGNQKNYLA

SEQ ID NO: 102 - protein, 7 aa, synthetic construct
GASTRES

SEQ ID NO: 103 - protein, 9 aa, synthetic construct
QNDHSYPYT

SEQ ID NO: 104 - protein, 116 aa, synthetic construct
QVQLQQPGPELVKPVASVKISCKTSDYAFSSSWMNWVKQRPGKGLEWIGWIYPGDGDTNYNGKFKGKATLTADKSSSTAYMQLSSLTSEDSAVYFCARGRWGMDNWGQGTSVTVSS

SEQ ID NO: 105 - protein, 10 aa, synthetic construct
DYAFSSSWMN

SEQ ID NO: 106 - protein, 17 aa, synthetic construct
WIYPGDGDTNYNGKFKG

SEQ ID NO: 107 - protein, 7 aa, synthetic construct
GRWGMDN

SEQ ID NO: 108 - protein, 107 aa, synthetic construct
DIVMTQSHKFMSTSVGDRVSITCKASQNVVTAVAWYQQKPGQSPKLLIYWASSRHTGVPDRFTGSGSGTDFTLTISNVQSEDLADYFCQQYSNYPFTFGSGTKLEIK

SEQ ID NO: 109 - protein, 11 aa, synthetic construct
KASQNVVTAVA

SEQ ID NO: 110 - protein, 7 aa, synthetic construct
WASSRHT

SEQ ID NO: 111 - protein, 9 aa, synthetic construct
QQYSNYPFT

SEQ ID NO: 112 - protein, 116 aa, synthetic construct
QVPLHQSGPELIKPGASLKISCKASDSAFNNSWMNWVRQRPGKGLEWIGWIYPGDDDTKYNGKFMGKATLTADKSSGTAYMQLSSLTSEDSAVYFCARGRWGMDYWGQGTSVTVSS

SEQ ID NO: 113 - protein, 10 aa, synthetic construct
DSAFNNSWMN

SEQ ID NO: 114 - protein, 17 aa, synthetic construct
WIYPGDDDTKYNGKFMG

SEQ ID NO: 115 - protein, 7 aa, synthetic construct
GRWGMDY

SEQ ID NO: 116 - protein, 107 aa, synthetic construct
DIVMTQFHNFMSTSVGDRVNINCKASQNVVTAVAWYQQKPGQSPKLLIYWASTRHTGVPDRFTGSGSGTDFTLTISNVQSEDLADYFCQQYSNYPFTFGSGTKLEIK

SEQ ID NO: 117 - protein, 11 aa, synthetic construct
KASQNVVTAVA

SEQ ID NO: 118 - protein, 7 aa, synthetic construct
WASTRHT

SEQ ID NO: 119 - protein, 9 aa, synthetic construct
QQYSNYPFT

SEQ ID NO: 120 - protein, 116 aa, synthetic construct
QVPLQQSGPELVKPGASVKISCKASDSAFNNSWTIWVRQRPGKGLEWIGWIYPGDDDTKYNGKFMGKATLTADKSSGTAYMQLSSLTSEDSAVYFCARGRWGMDYWGQGTSVTVSS

SEQ ID NO: 121 - protein, 10 aa, synthetic construct
DSAFNNSWTI

SEQ ID NO: 122 - protein, 17 aa, synthetic construct
WIYPGDDDTKYNGKFMG

SEQ ID NO: 123 - protein, 7 aa, synthetic construct
GRWGMDY

SEQ ID NO: 124 - protein, 107 aa, synthetic construct
DIVMTQSRKFMSTSVGDRVSISCKASQNVVTAVAWYQQKPGQSPKLLIYWASTRHTGVPDRFTGSGSGTDFTLTISNVQSEDLADYFCQQYSNYPFTFGSGTELEIK

SEQ ID NO: 125 - protein, 11 aa, synthetic construct
KASQNVVTAVA

SEQ ID NO: 126 - protein, 7 aa, synthetic construct
WASTRHT

SEQ ID NO: 127 - protein, 9 aa, synthetic construct
QQYSNYPFT

SEQ ID NO: 128 - protein, 122 aa, synthetic construct
QVQLVQSGAEVKKPGSSVKVSCKASGYTFTSYNMHWVRQAPGQGLEWMGYIYPGNGGTNYNQKFKGRVTITADESTSTAYMELSSLRSEDTAVYYCARGASMITTGAWFAYWGQGTLVTVSS

SEQ ID NO: 129 - protein, 122 aa, synthetic construct
QVQLVQSGAEVKKPGSSVKVSCKASGYTFTSYNMHWVRQAPGQGLEWMGYIYPGNGGTNYNQKFKGRVTITADTSTSTAYMELSSLRSEDTAVYYCARGASMITTGAWFAYWGQGTLVTVSS

SEQ ID NO: 130 - protein, 122 aa, synthetic construct
QVQLVQSGAEVKKPGSSVKVSCKASGYTFTSYNMHWVRQAPGQGLEWMGYIYPGSGGTNYNQKFKGRVTITADESTSTAYMELSSLRSEDTAVYYCARGASMITTGAWFAYWGQGTLVTVSS

SEQ ID NO: 131 - protein, 122 aa, synthetic construct
QVQLVQSGAEVKKPGSSVKVSCKASGYTFTSYNMHWVRQAPGQGLEWMGYIYPGNGGTSYNQKFKGRVTITADESTSTAYMELSSLRSEDTAVYYCARGASMITTGAWFAYWGQGTLVTVSS

SEQ ID NO: 132 - protein, 122 aa, synthetic construct
QVQLVQSGAEVKKPGSSVKVSCKASGYTFTSYNMHWVRQAPGQGLEWMGYIYPGSGGTSYNQKFKGRVTITADESTSTAYMELSSLRSEDTAVYYCARGASMITTGAWFAYWGQGTLVTVSS

SEQ ID NO: 133 - protein, 111 aa, synthetic construct
DIQMTQSPSSVSASVGDRVTITCRASESLDTYGNSFMYWYQQKPGKAPKLLIYLASSLESGVPSRFSGSGSGTDFTLTISSLQPEDFATYYCHQNHEDPWTFGQGTKVEIK

SEQ ID NO: 134 - protein, 17 aa, synthetic construct
YIYPGSGGTNYNQKFKG

SEQ ID NO: 135 - protein, 17 aa, synthetic construct
YIYPGSGGTSYNQKFKG

SEQ ID NO: 136 - protein, 117 aa, synthetic construct
QVQLVQSGAEVKKPGSSVKVSCKASGYTFTNHWIEWVRQAPGQGLEWMGDIFPGGGYTDYNEKFKGRVTITADESTSTAYMELSSLRSEDTAVYYCARGRDLAMDYWGQGTLVTVSS

SEQ ID NO: 137 - protein, 117 aa, synthetic construct
QVQLVQSGAEVKKPGSSVKVSCKASGYTFTNHWIEWVRQAPGQGLEWIGDIFPGGGYTDYNEKFKGRATITADESTSTAYMELSSLRSEDTAVYYCARGRDLAMDYWGQGTLVTVSS

SEQ ID NO: 138 - protein, 107 aa, synthetic construct
DIQMTQSPSSLSASVGDRVTITCHASQNINVWLGWYQQKPGKAPKLLIYKASNLHTGVPSRFSGSGSGTDFTLTISSLQPEDFATYYCQQGQSYPYTFGQGTKVEIK

SEQ ID NO: 139 - protein, 107 aa, synthetic construct
DIQMTQSPSSLSASVGDRVTITCRASQNINVWLGWYQQKPGKAPKLLIYKASNLHTGVPSRFSGSGSGTDFTLTISSLQPEDFATYYCQQGQSYPYTFGQGTKVEIK

SEQ ID NO: 140 - protein, 107 aa, synthetic construct
DIQMTQSPSSLSASVGDRVTITCSASQNINVWLGWYQQKPGKAPKLLIYKASNLHTGVPSRFSGSGSGTDFTLTISSLQPEDFATYYCQQGQSYPYTFGQGTKVEIK

SEQ ID NO: 141 - protein, 107 aa, synthetic construct
DIQMTQSPSSLSASVGDRVTITCGASQNINVWLGWYQQKPGKAPKLLIYKASNLHTGVPSRFSGSGSGTDFTLTISSLQPEDFATYYCQQGQSYPYTFGQGTKVEIK

SEQ ID NO: 142 - protein, 107 aa, synthetic construct
DIQMTQSPSSLSASVGDRVTITCEASQNINVWLGWYQQKPGKAPKLLIYKASNLHTGVPSRFSGSGSGTDFTLTISSLQPEDFATYYCQQGQSYPYTFGQGTKVEIK

SEQ ID NO: 143 - protein, 11 aa, synthetic construct
RASQNINVWLG

SEQ ID NO: 144 - protein, 11 aa, synthetic construct
SASQNINVWLG

SEQ ID NO: 145 - protein, 11 aa, synthetic construct
GASQNINVWLG

SEQ ID NO: 146 - protein, 11 aa, synthetic construct
EASQNINVWLG

SEQ ID NO: 147 - protein, 116 aa, synthetic construct
QITLKESGPTLVKPTQTLTLTCTASGFDFSRSWMNWIRQPPGKALEWLAEINPDSSTINYTTSLKDRFTISRDTSKNQVVLTMTNMDPVDTATYYCARYGNWFPYWGQGTLVTVSS

SEQ ID NO: 148 - protein, 116 aa, synthetic construct
EVQLLESGGGLVQPGGSLRLSCAASGFDFSRSWMNWVRQAPGKGLEWVSEINPDSSTINYATSLKDRFTISRDNSKNTLYLQMNSLRAEDTAVYYCARYGNWFPYWGQGTLVTVSS

SEQ ID NO: 149 - protein, 116 aa, synthetic construct
QLQLQESGPGLVKPSETLSLTCTASGFDFSRSWMNWIRQPPGKGLEWIGEINPDSSTINYATSLKDRFTISRDTSKNQLSLKLSSVTAADTAVYYCARYGNWFPYWGQGTLVTVSS

SEQ ID NO: 150 - protein, 107 aa, synthetic construct
AIQLTQSPSSLSASVGDRVTITCKASQNVDTNVAWYQQKPGKAPKALIYSASYRYSGVPSRFSGSGSGTDFTLTISSLQPEDFATYYCQQYDSYPLTFGQGTKVEIK

SEQ ID NO: 151 - protein, 107 aa, synthetic construct
DIQMTQSPSSLSASVGDRVTITCKASQNVDTNVAWYQQKPGKAPKALIYSASYRYSGVPSRFSGSGSGTDFTLTISSLQPEDFATYYCQQYDSYPLTFGQGTKVEIK

SEQ ID NO: 152 - protein, 107 aa, synthetic construct
DIQMTQSPSSLSASVGDRVTITCRASQNVDTNVAWYQQKPGKAPKALIYSASYRYSGVPSRFSGSGSGTDFTLTISSLQPEDFATYYCQQYDSYPLTFGQGTKVEIK

SEQ ID NO: 153 - protein, 107 aa, synthetic construct
DIQMTQSPSSLSASVGDRVTITCSASQNVDTNVAWYQQKPGKAPKALIYSASYRYSGVPSRFSGSGSGTDFTLTISSLQPEDFATYYCQQYDSYPLTFGQGTKVEIK

SEQ ID NO: 154 - protein, 107 aa, synthetic construct
DIQMTQSPSSLSASVGDRVTITCGASQNVDTNVAWYQQKPGKAPKALIYSASYRYSGVPSRFSGSGSGTDFTLTISSLQPEDFATYYCQQYDSYPLTFGQGTKVEIK

SEQ ID NO: 155 - protein, 107 aa, synthetic construct
DIQMTQSPSSLSASVGDRVTITCEASQNVDTNVAWYQQKPGKAPKALIYSASYRYSGVPSRFSGSGSGTDFTLTISSLQPEDFATYYCQQYDSYPLTFGQGTKVEIK

SEQ ID NO: 156 - protein, 119 aa, synthetic construct
EVQLVESGGGLVQPGGSLRLSCAVSGFSLISYGVHWVRQAPGKGLEWLGVIWRGGSTDYNAAFMSRLTISKDNSKNTVYLQMNSLRAEDTAVYYCAKTLITTGYAMDYWGQGTLVTVSS

SEQ ID NO: 157 - protein, 119 aa, synthetic construct
QVQLQESGPGLVKPSETLSLTCTVSGFSLISYGVHWVRQPPGKGLEWLGVIWRGGSTDYNAAFMSRLTISKDTSKNQVSLKLSSVTAADTAVYYCAKTLITTGYAMDYWGQGTLVTVSS

SEQ ID NO: 158 - protein, 119 aa, synthetic construct
QVQLQESGPGLVKPSDTLSLTCAVSGFSLISYGVHWVRQPPGKGLEWLGVIWRGGSTDYNAAFMSRLTMSKDTSKNQVSLKLSSVTAVDTAVYYCAKTLITTGYAMDYWGQGTLVTVSS

SEQ ID NO: 159 - protein, 106 aa, synthetic construct
DIQMTQSPSSLSASVGDRVTITCKASEDIYNRLAWYQQKPGKAPKLLISGATSLETGVPSRFSGSGSGTDYTLTISSLQPEDFATYYCQQYWSTPTFGQGTKVEIK

SEQ ID NO: 160 - protein, 106 aa, synthetic construct
DIQMTQSPSSLSASVGDRVTITCKASEDIYNRLAWYQQKPGKAPKLLISGATSLETGVPSRFSGSGSGTDYTFTISSLQPEDIATYYCQQYWSTPTFGQGTKVEIK

SEQ ID NO: 161 - protein, 106 aa, synthetic construct
EIVLTQSPDFQSVTPKEKVTITCKASEDIYNRLAWYQQKPDQSPKLLISGATSLETGVPSRFSGSGSGTDYTLTINSLEAEDAATYYCQQYWSTPTFGQGTKVEIK

SEQ ID NO: 162 - protein, 485 aa, synthetic construct
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 214 aa, synthetic construct
DIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 445 aa, synthetic construct
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 165 - protein, 585 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 166 - protein, 241 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIK

SEQ ID NO: 167 - protein, 393 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 168 - protein, 393 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 169 - protein, 993 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 170 - protein, 993 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 171 - protein, 249 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 172 - protein, 1001 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 173 - protein, 249 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 174 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 175 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 176 - protein, 1001 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 177 - protein, 1001 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 178 - protein, 249 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 179 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 180 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 181 - protein, 1001 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 182 - protein, 1001 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 183 - protein, 246 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 184 - protein, 398 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 185 - protein, 398 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 186 - protein, 846 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 187 - protein, 998 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 188 - protein, 246 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 189 - protein, 398 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 190 - protein, 398 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 191 - protein, 998 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 192 - protein, 998 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 193 - protein, 246 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 194 - protein, 398 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 195 - protein, 398 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 196 - protein, 998 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 197 - protein, 998 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 198 - protein, 575 aa, synthetic construct
QVQLVQSGAEVKKPGSSVKVSCKASGYTFTSYNMHWVRQAPGQGLEWMGYIYPGNGGTNYNQKFKGRVTITADESTSTAYMELSSLRSEDTAVYYCARGASMITTGAWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 199 - protein, 218 aa, synthetic construct
DIQMTQSPSSVSASVGDRVTITCRASESLDTYGNSFMYWYQQKPGKAPKLLIYLASSLESGVPSRFSGSGSGTDFTLTISSLQPEDFATYYCHQNHEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 401 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 201 - protein, 17 aa, synthetic construct
YIYPGNGGTSYNQKFKG

(19) United States
(12) Patent Application Publication
(10) Pub. No.: US 2026/0070999 A1
(43) Pub. Date: Mar. 12, 2026

(54) METHODS OF TREATING AUTOIMMUNE DISORDERS USING MULTIMERIC ANTI-CD38/ANTI-CD3 ANTIBODIES

(71) Applicant: IGM Biosciences, Inc., Mountain View, CA (US)

(72) Inventors: Carrie SAULSBERY, West Chester, PA (US); Mary Beth HARLER, Mountain View, CA (US)

(21) Appl. No.: 19/106,184

(22) PCT Filed: Dec. 21, 2023
(86) PCT No.: PCT/US2023/085568

§ 371 Date: Feb. 24, 2025

Related U.S. Application Data
(60) Provisional application No. 63/476,891, filed on Dec. 22, 2022.
(60) Provisional application No. 63/488,047, filed on Mar. 2, 2023.

(51) Int. Cl. C07K 16/28

(52) U.S. Cl. CPC C07K 16/2896; C07K 16/2809; C07K 2317/31; C07K 2317/622; C07K 2317/73

(57) ABSTRACT

This disclosure provides methods of treating an autoimmune disorder comprising administering to a subject in need of treatment an effective amount of a multimeric bispecific anti-CD38/anti-CD3 antibody comprising five bivalent binding units and a modified J chain. For example, the autoimmune disorder is, in some embodiments, a disorder wherein CD38-expressing cells contribute to chronic inflammation in the subject.

### CROSS-REFERENCE TO RELATED APPLICATIONS

This application claims the benefit of U.S. Provisional Patent Application No. 63/476,891, filed 22 Dec. 2022 and U.S. Provisional Patent Application No. 63/488,047, filed 2 Mar. 2023, both of which are incorporated herein by reference in their entireties.

### SEQUENCE LISTING

The instant application contains a Sequence Listing which has been submitted electronically in XML format and is hereby incorporated by reference in its entirety. The XML copy was created on 21 Dec. 2023, is named 064WO1-Sequence-Listing.xml, and is 202 kilobytes in size.

### BACKGROUND

Autoimmune disorders are characterized by exaggerated immune responses and/or loss of immune tolerance for self-antigens. Such disorders have traditionally been treated with immunosuppressive agents. The anti-CD20 antibody, rituximab, which reduces the number of CD20+B cells resulting in a reduction in pathogenic autoantibodies and leads to improvement in clinical outcomes, has been approved by the United States Food and Drug Administration (US FDA) for treating the autoimmune disorders: rheumatoid arthritis (RA), granulomatosis with polyangiitis (GPA), microscopic polyangiitis (MPA), pemphigus vulgaris (PV), and has been used for a variety of other autoimmune disorders off-label. See, e.g., RITUXAN® Prescribing Information, Revised December 2021 and Randall, Aust Prescr 39:131-34 (2016). However, rituximab does not result in substantial depletion of B cells in secondary lymphoid organs, which limits its effectiveness. See Kamburova, American Journal of Transplantation 13:1503-11 (2013). Other approved therapies targeting B-cell related autoimmune disorders include the anti-B-lymphocyte stimulator (BlyS) antibody, belimumab (sold under the tradename, BENLYSTA®, US FDA approved for treating systemic lupus erythematosus (SLE) (see BENLYSTA® Prescribing Information, revised July 2022), infliximab (sold under the tradename, REMICADE®), US FDA approved for treating rheumatoid arthritis (see REMICADE® Prescribing Information, revised October 2021), adalimumab (sold under the tradename, HUMIRA®), US FDA approved for treating rheumatoid arthritis (see HUMIRA® Prescribing Information, revised February 2021), and etanercept (sold under the tradename, ENBREL®), US FDA approved for treating rheumatoid arthritis (See ENBREL® Prescribing Information, revised December 2012).

The glycoprotein, CD38, is expressed in a variety of tumor cells, such as various hematologic malignancies including chronic lymphocytic leukemia (CLL), multiple myeloma (MM), Hodgkin's lymphoma (HL), diffuse large B-cell lymphoma (DLBCL), peripheral T cell lymphoma (PTCL), and various solid tumors, including prostate cancer, non-small cell lung cancer (NSCLC), squamous cell carcinoma of the head and neck (SCCHN), ovarian cancer, and liver cancer, and is therefore considered a potential target for directed therapeutics (Martin et al., Cells 8:1522 (2019)).

Two anti-CD38 IgG antibodies, daratumumab and isatuximab, have been approved for the treatment of multiple myeloma (MM), the second most common blood cancer, representing ten percent of all blood cancers. Despite the development of these and other treatments, MM remains incurable with only a forty percent 5-year survival rate. Most MM patients become resistant to treatment over the course of therapy (Marzo et al., Oncotarget 7 (37): 60698-711 (2016)).

CD38 likewise affects inflammation, regulating, e.g., transmigration and chemotaxis of neutrophils and monocytes to sites of inflammation, phagocytosis, and cytokine release during microbial infections. See, e.g., Piedra-Quintero et al., Front. Immunol 11:597959, doi: 10.3389/fimmu.2020.597959 (2020). Substantial evidence also implicates CD38 as having a role in the development of autoimmune diseases, including rheumatoid arthritis (RA), multiple sclerosis (MS), and systemic lupus erythematosus (SLE). Id. Daratumumab is currently under clinical investigation for the treatment of certain autoimmune diseases including idiopathic thrombocytopeniaurpura (ITP) (Clinicaltrials.gov identifier NCT04703621), primary immune thrombocytopenia (Clinicaltrials.gov identifier NCT05562882), lupus nephritis (Clinicaltrials.gov identifier NCT04868838), and autoimmune antibody mediated hemolytic anemia (Clinicaltrials.gov identifier NCT05004259). Isatuximab is currently under clinical investigation for the treatment of certain autoimmune diseases including, for example, warm autoimmune hemolytic anemia (Clinicaltrials.gov identifier NCT04661033).

Antibodies that can multimerize, such as IgA and IgM antibodies, have emerged as promising drug candidates in the fields of, e.g., immuno-oncology, autoimmunity, inflammation, and infectious diseases, allowing for improved specificity, improved avidity, and the ability to bind to multiple binding targets. See, e.g., U.S. Pat. Nos. 9,951,134, 9,938,347, 10,570,191, 10,604,559, 10,618,978, 10,787,520, 10,899,835, and 11,639,389, U.S. Patent Application Publication No. 2019-0338041, and PCT Application Publication No. WO 2023/150677, the contents of which are incorporated herein by reference in their entireties.

There remains a need for improved autoimmune and inflammatory disorder therapies that can effectively reduce immune cell populations, e.g., B cell populations, with minimal toxicity. This disclosure addresses these and other needs.

### SUMMARY

Provided herein is a method of treating an autoimmune disorder comprising administering to a subject in need of treatment an effective amount of a multimeric bispecific anti-CD38/anti-CD3 antibody. The multimeric bispecific anti-CD38/anti-CD3 antibody comprises five bivalent binding units and a modified J chain. Each binding unit comprises two IgM heavy chains, each comprising a heavy chain variable region (VH) and an IgM constant region or multimerizing fragment or variant thereof, and two light chains, each comprising a light chain variable region (VL) and a light chain constant region. The VH comprises three immunoglobulin complementarity determining regions: HCDR1, HCDR2, and HCDR3, and the VL comprises three immunoglobulin complementarity determining regions: LCDR1, LCDR2, and LCDR3, where the HCDR1, HCDR2, HCDR3, LCDR1, LCDR2, and LCDR3 comprise, respectively, the amino acid sequences SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 201, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95, where the modified J chain comprises a J chain or functional fragment or variant thereof indirectly or directly fused to an scFv molecule that specifically binds to CD3.

In some embodiments, the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71.

In some further embodiments, the VH and VL each comprise, respectively, an amino acid sequence that is at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142.

In some embodiments, the VH and VL each comprise, respectively, an amino acid sequence that is at least 80%, 85%, 90%, 95%, or 100% identical to the amino acid sequence of SEQ ID NO: 128 and SEQ ID NO: 133. In some embodiments, the VH and VL comprise, respectively, the amino acid sequence of SEQ ID NO: 128 and SEQ ID NO: 133.

In yet some additional embodiments, the IgM heavy chain constant region or multimerizing fragment or variant thereof comprises a Cμ4 region and a μ tailpiece region. In some embodiments, the IgM heavy chain constant region or multimerizing fragment or variant thereof further comprises a Cμ1 region, a Cμ2 region, Cμ3 region, or any combination thereof. In some embodiments, the IgM heavy chain constant region or multimerizing fragment or variant thereof is a human IgM constant region. In some embodiments, the heavy chain comprises the amino acid sequence SEQ ID NO: 198 and the light chain comprises the amino acid sequence SEQ ID NO: 199.

Also provided herein is a method of treating an autoimmune disorder comprising administering to a subject in need of treatment an effective amount of a multimeric bispecific anti-CD38/anti-CD3 antibody, which comprises five bivalent binding units and a modified J chain, where each binding unit comprises two IgM heavy chains, each comprising a heavy chain variable region (VH) and an IgM constant region or multimerizing fragment or variant thereof, and two light chains, each comprising a light chain variable region (VL) and a light chain constant region, where the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161, and where the modified J chain comprises a J chain or functional fragment or variant thereof indirectly or directly fused to an scFv molecule that specifically binds to CD3.

In some embodiments, the scFv molecule that specifically binds to CD3 comprises a heavy chain variable region (VH) and a light chain variable region (VL), where the VH comprises VH complementarity-determining regions VHCDR1, VHCDR2, and VHCDR3 and the VL comprises VL complementarity-determining regions VLCDR1, VLCDR2, and VLCDR3, where the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49. In some embodiments, the VHCDR1, VHCDR2, VHCDR3, VLCDR1, VLCDR2, and VLCDR3 comprise, respectively, the amino acid sequences SEQ ID NO: 25, SEQ ID NO: 26, SEQ ID NO: 27, SEQ ID NO: 29, SEQ ID NO: 30, and SEQ ID NO: 31.

In some additional embodiments, the VH and VL of the scFv each comprise, respectively, an amino acid sequence that is at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; SEQ ID NO: 46 and SEQ ID NO: 48; or SEQ ID NO: 50 and SEQ ID NO: 52. In some embodiments, the scFv comprises the VH and VL amino acid sequences SEQ ID NO: 24 and SEQ ID NO: 28, respectively. In some embodiments, the scFv comprises the amino acid sequence of SEQ ID NO: 166, SEQ ID NO: 171, SEQ ID NO: 173, SEQ ID NO: 178, SEQ ID NO: 183, SEQ ID NO: 188, or SEQ ID NO: 193. In some embodiments, the scFv comprises the amino acid sequence of SEQ ID NO: 173.

In yet some further embodiments, the modified J chain comprises the native human J chain amino acid sequence SEQ ID NO: 7. In some embodiments, the modified J chain comprises SEQ ID NO: 167, SEQ ID NO: 174, SEQ ID NO: 179, SEQ ID NO: 184, SEQ ID NO: 189, or SEQ ID NO: 194. In some embodiments, the modified J chain further comprises human serum albumin indirectly or directly fused to the J chain or functional fragment or variant thereof. In some embodiments, the modified J chain comprises SEQ ID NO: 169, SEQ ID NO: 176, SEQ ID NO: 181, SEQ ID NO: 186, SEQ ID NO: 191, or SEQ ID NO: 196.

In some additional embodiments, the modified J chain or functional fragment or variant thereof comprises an alanine (A) substitution at the amino acid position corresponding to amino acid Y102 of SEQ ID NO: 7. In some embodiments, the modified J chain or functional fragment or variant thereof comprises the amino acid sequence SEQ ID NO: 8. In some embodiments, the modified J chain comprises SEQ ID NO: 168, SEQ ID NO: 175, SEQ ID NO: 180, SEQ ID NO: 185, SEQ ID NO: 190, or SEQ ID NO: 195. In some embodiments, the modified J chain comprises SEQ ID NO: 175. In some embodiments, the modified J chain further comprises human serum albumin indirectly or directly fused to the J chain or functional fragment or variant thereof. In some embodiments, the modified J chain comprises SEQ ID NO: 170, SEQ ID NO: 177, SEQ ID NO: 182, SEQ ID NO: 187, SEQ ID NO: 192, or SEQ ID NO: 197.

In some particular embodiments, the IgM heavy chains each comprise the amino acid sequence SEQ ID NO: 198, the light chains each comprise the amino acid sequence SEQ ID NO: 199, and the modified J chain comprises the amino acid sequence SEQ ID NO: 175.

In some embodiments, the autoimmune disorder is a disorder where CD38-expressing cells contribute to chronic inflammation in the subject. In some embodiments, the autoimmune disorder is systemic lupus erythematosus (SLE), antiphospholipid syndrome (APS), idiopathic thrombocytopeniaurpura (ITP), warm autoimmune hemolytic anemia (wAIHA), multiple sclerosis (MS), myasthenia gravis (MG), pemphigus vulgaris (PV), anti-neutrophil cytoplasmic autoantibody (ANCA) associated vasculitis (AAV), thyroid eye disease (TED), membranous glomerulonephritis (MGN), Neuromyelitis optica (NMO), Guillain-Barré syndrome (GBS), chronic inflammatory demyelinating polyradiculoneuropathy (CIDP), IgA nephropathy, Goodpasture's syndrome, granulomatosis with polyangiitis (GPA), microscopic polyangiitis (MPA), Sjögren syndrome, Behcet's disease, alopecia areata, immunoglobulin G4-related disease (IgG4-RD), phospholipase A2 receptor-associated membranous nephropathy (PLA2R MN), myositis, type 1 diabetes, systemic sclerosis, or rheumatoid arthritis (RA).

In some embodiments, the autoimmune disorder is SLE. In some embodiments, the SLE comprises lupus nephritis (LN). In some embodiments, the autoimmune disorder is RA. In some embodiments, the autoimmune disorder is MG.

In some further embodiments, the subject had previously been treated with a biologic autoimmune disorder treatment. In some embodiments, the subject had previously been treated with an anti-CD20 antibody, anti-B-lymphocyte stimulator (BlyS) antibody, or a TNF inhibitor. In some embodiments, the autoimmune disorder is SLE, and the subject had previously been treated with belimumab. In some embodiments, the autoimmune disorder is MS, and the subject had previously been treated with rituximab. In some embodiments, the autoimmune disorder is RA, and the subject had previously been treated with infliximab, adalimumab, or etanercept.

In some embodiments, the administration of the multimeric bispecific anti-CD38/anti-CD3 antibody comprises intravenous infusion. In some other embodiments, the administration of the multimeric bispecific anti-CD38/anti-CD3 antibody comprises subcutaneous injection. In some embodiments, the subject is a human.

### BRIEF DESCRIPTION OF THE DRAWINGS/FIGURES

FIG. 1 shows T cell fratricide levels for in vitro treatment with exemplary antibody, IgM B-2, or CD38×CD3 IgG for CD4+ and CD8+ T cells as described in Example 2.

FIG. 2 shows the percentage of CD38+CD19+B cell killing after treatment with various concentrations of the exemplary antibody, IgM B-2, in a T cell dependent cellular cytotoxicity (TDCC) assay on peripheral blood mononuclear cells (PBMCs) from one healthy donor, two biologic naïve systemic lupus erythematosus (SLE) donors, one biologic experienced SLE donor, and one rheumatoid arthritis (RA) donor as described in Example 3.

FIGS. 3A-3D show the concentration of IFNγ (FIG. 3A), IL-2 (FIG. 3B), IL-6 (FIG. 3C), and TNFα (FIG. 3D) in the supernatants of healthy (closed circle), SLE biologic naïve (closed square and open circle), SLE biologic experienced (open square), and RA (triangle) PBMCs following 72 hour treatment with IgM-B-2 as described in Example 3.

FIGS. 4A-4D show the number of activated CD4+ T cells (CD19-CD3+CD4+CD69+) (FIG. 4A), CD8+ T cells (CD19-CD3+CD8+CD69+) (FIG. 4B), peripheral NK cells (CD19-CD56^{bright}) (FIG. 4C), and hematic NK cells (CD19-CD56^{dim}) (FIG. 4D) in peripheral blood mononuclear cells (PBMCs) from one healthy donor (closed circle), two biologic naïve systemic lupus erythematosus (SLE) donors (closed square and open circle), one biologic experienced SLE donor (open square), and one rheumatoid arthritis (RA) donor (triangle) after treatment with IgM-B-2 as described in Example 3.

FIG. 5 shows the percent of killing of CD138+CD319+ plasma cells in bone marrow mononuclear cells isolated from four healthy donors after treatment with various concentrations of IgM B-2 in a T cell dependent cellular cytotoxicity (TDCC) assay as described in Example 6.

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term, “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, P-S (ed.), 2nd ed., 2002, CRC Press; The Dictionary of Cell and Molecular Biology, Lacki, J. M. (ed.) 5th ed., 2013, Academic Press; and the Oxford Dictionary of Biochemistry and Molecular Biology, Cammack, R., et al., (eds.) 2d ed., 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 2 or more, 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine. Asparagine (N)-linked glycans are described in more detail elsewhere in this disclosure.

An “isolated” polypeptide or a fragment, variant, or derivative thereof is intended to refer to a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or a grammatical variant thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptide which retains at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide, include fragments as described above, and also polypeptides with altered amino acid sequences due to amino acid substitutions, deletions, and/or insertions. In certain embodiments, variants can be non-naturally occurring. Non-naturally occurring variants can be produced using art-known mutagenesis techniques. Variant polypeptides can comprise conservative or non-conservative amino acid substitutions, deletions, and/or additions. Derivatives are polypeptides that have been altered to possess and/or exhibit additional features not found on the original polypeptide. Examples include fusion proteins. As used herein, a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody as described in detail herein that retains antigen-specific binding. In certain embodiments, a “binding molecule” is an antibody as provided herein.

As used herein, the terms “binding domain” or “antigen-binding domain” (which can be used interchangeably) refer to a region of a binding molecule, e.g., an antibody, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule,” e.g., an “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Greenfield, E. A. (ed.), Antibodies: A Laboratory Manual, Second Edition, Cold Spring Harbor Laboratory Press, 2014). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four or eight complete heavy chains and four or eight complete light chains and includes a J chain or functional fragment or variant thereof, and/or a secretory component, or an IgM binding molecule, e.g., an IgM antibody or IgM-like antibody, that includes ten or twelve complete heavy chains and ten or twelve complete light chains and optionally includes a J chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG1, IgG2, IgG3, IgG4, IgAQ1, IgA2, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

“Binding unit” is used herein to refer to the portion of a binding molecule, e.g., an antibody, which corresponds to a standard immunoglobulin structure, e.g., an “H2L2” immunoglobulin structure of two heavy chains and two light chains, or, for certain heavy chain-only antibodies an “H2” immunoglobulin structure. In certain embodiments, e.g., where the binding molecule is a bivalent IgG antibody, the terms “binding molecule” and “binding unit” are equivalent. Such a binding molecule is also referred to herein as “monomeric.” In other embodiments, e.g., where the binding molecule is a “multimeric binding molecule,” e.g., a dimeric or tetrameric IgA antibody or a pentameric or hexameric IgM antibody, the binding molecule comprises two or more “binding units”-two in the case of an IgA dimer, four in the case of an IgA tetramer, five in the case of an IgM pentamer, or six in the case of an IgM hexamer. A binding unit need not include full-length antibody heavy and light chains, but will typically be bivalent, i.e., will include two “antigen-binding domains,” as defined above. As used herein, certain examples of a binding molecule provided in this disclosure are “dimeric,” and include two bivalent binding units that include IgA constant regions or multimerizing fragments thereof. Certain examples of a binding molecule provided in this disclosure are “pentameric” or “hexameric,” and include five or six bivalent binding units that include IgM constant regions or multimerizing fragments or variants thereof. A binding molecule, e.g., an antibody comprising two or more, e.g., two, five, or six binding units, is referred to herein as “multimeric.”

The term “J chain” as used herein refers to the J chain of IgM or IgA antibodies of any animal species, any functional fragment thereof, derivative thereof, and/or variant thereof, including a mature human J chain, the amino acid sequence of which is presented as SEQ ID NO: 7. Various J chain variants and modified J chain derivatives are available, e.g., in U.S. Pat. No. 10,899,835. As persons of ordinary skill in the art will recognize, “a functional fragment” or “a functional variant” includes those fragments and variants that can associate with IgM heavy chain constant regions to form a pentameric IgM antibody, or associate with IgA heavy chain constant regions to form a dimeric or tetrameric IgA antibody.

The term “modified J chain” is used herein to refer to a derivative of a J chain polypeptide comprising a heterologous moiety, e.g., a heterologous polypeptide, e.g., an extraneous binding domain or functional domain introduced into or attached to the J chain sequence. The introduction can be achieved by any means, including direct or indirect fusion of the heterologous polypeptide or other moiety or by attachment through a peptide or chemical linker. The term “modified human J chain” encompasses, without limitation, a native human J chain comprising the amino acid sequence of SEQ ID NO: 7 or a functional fragment thereof, or functional variant thereof, modified by the introduction of a heterologous moiety, e.g., a heterologous polypeptide, e.g., an extraneous binding domain. In certain embodiments, the heterologous moiety does not interfere with efficient polymerization of IgM into a pentamer or IgA into a multimer, e.g., a dimer or tetramer, and binding of such polymers to a target. Exemplary modified J chains can be found, e.g., in U.S. Pat. Nos. 9,951,134, 10,400,038, and 10,618,978, and 11,639,389, each of which is incorporated herein by reference in its entirety.

The terms “valency,” “bivalent,” “multivalent” and grammatical equivalents, refer to the number of binding domains, e.g., antigen-binding domains in a given antibody or in a given binding unit. As such, the terms “bivalent”, “tetravalent”, and “hexavalent” in reference to a given antibody, denote the presence of two antigen-binding domains, four antigen-binding domains, and six antigen-binding domains, respectively. A typical IgM antibody, where each binding unit is bivalent, can have 10 or 12 valencies. A bivalent or multivalent binding molecule, e.g., antibody, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by an antibody, such as an scFv fragment. A target can be, e.g., a polypeptide, a nucleic acid, a carbohydrate, a lipid, or other molecule. Moreover, a “target” can, for example, be a cell, an organ, or an organism that comprises an epitope that can be bound by an antibody, such as an scFv fragment. As used herein, a “target antigen” is a target molecule, e.g., a polypeptide, a nucleic acid, a carbohydrate, a lipid, or other molecule that can be bound by an antibody, such as an scFv fragment as provided herein. In certain embodiments a target antigen can appear on the surface of a cell, e.g., a tumor cell. A “tumor-specific antigen” as used herein is a protein or other cell surface target antigen that is unique to tumor cells, at least at later stages of development of the organism. As used herein, a “tumor-associated antigen” is a protein or other cell surface target antigen that is not necessarily unique to tumor cells but is typically expressed much more abundantly and/or at higher density on tumor cells than on normal, healthy cells.

Both the light and heavy chains of antibodies, such as an scFv fragment, are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains comprise the carboxy-terminus of the heavy and light chain, respectively.

A “full length IgM antibody heavy chain” is a polypeptide that includes, in an N-terminal to C terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a μ tailpiece.

As used herein, “IgM-like antibody” refers to a binding molecule that includes at least a multimerizing fragment or variant of an IgM heavy chain constant region that retains the ability to form hexamers or pentamers, e.g., in association with a J chain. An IgM-like antibody typically includes at least the Cμ4 and IgM tail-piece (tp) domains of an IgM constant region but can include heavy chain constant region domains from other antibody isotypes, e.g., IgG, from the same species or from a different species. An IgM-like antibody can likewise be an antibody fragment in which one or more constant region domains are deleted, as long as the IgM-like antibody is capable of multimerizing into a hexamer and/or a pentamer. Thus, an IgM-like antibody can be, e.g., a hybrid IgM/IgG antibody or can be a “multimerizing fragment” of an IgM antibody.

As indicated above, variable region(s) allow an antibody such as an scFv fragment to selectively recognize and specifically bind epitopes on antigens. That is, the VL domain and VH domain, or VHH domain, or subset of the complementarity determining regions (CDRs), of an antibody combine to form the antigen-binding domain. More specifically, an antigen-binding domain can be defined by three CDRs on each of the VH and VL chains. Certain antibodies form larger structures. For example, an IgA can form a molecule that includes two or four H2L2 binding units and a J chain covalently attached via disulfide bonds, which can be further associated with a secretory component; an IgM can form a pentameric or hexameric molecule that includes five or six H2L2 binding units and optionally a J chain covalently attached via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Other overlapping CDR definitions can be found, e.g., in Al-Lazikani B. et al., J. Mol. Biol. 273:927-948 (1997); MacCallum et al., J. Mol. Biol. 262:732-745 (1996); Abhinandan and Martin, Mol. Immunol. 45:3832-3839 (2008); Lefranc M. P. et al., Dev. Comp. Immunol. 27:55-77 (2003); and Honegger and Plückthun, J. Mol. Biol. 309:657-670 (2001), which are incorporated herein by reference in their entireties. Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res. 36: W503-508, 2008). Application of any particular definition (or other definitions known to those of ordinary skill in the art) to refer to a CDR of an antibody or variant thereof is intended to be within the scope of the term as defined and used herein, unless otherwise indicated. The appropriate amino acids which encompass the “Kabat” and “Chothia” CDRs as defined by the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J chain, T cell Receptors for Antigen, T cell Surface Antigens, B-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine(S) (SEQ ID NO: 1) or glycine (G) (SEQ ID NO: 2)):

Sequential (SEQ ID NO: 1 or SEQ ID NO: 2)/KABAT numbering key for IgM heavy chain

| | |
|---|---|
| 1/127 | GSASAPTLFP LVSCENSPSD TSSVAVGCLA QDFLPDSITF |
| | SWKYKNNSDI |
| 51/176 | SSTRGFPSVL RGGKYAATSQ VLLPSKDVMQ GTDEHVVCKV |
| | QHPNGNKEKN |
| 101/226 | VPLPVIAELP PKVSVFVPPR DGFFGNPRKS KLICQATGFS |
| | PRQIQVSWLR |
| 151/274 | EGKQVGSGVT TDQVQAEAKE SGPTTYKVTS TLTIKESDWL |
| | XQSMFTCRVD |
| 201/324 | HRGLTFQQNA SSMCVPDQDT AIRVFAIPPS FASIFLTKST |
| | KLTCLVTDLT |
| 251/374 | TYDSVTISWT RQNGEAVKTH TNISESHPNA TFSAVGEASI |
| | CEDDWNSGER |
| 301/424 | FTCTVTHTDL PSPLKQTISR PKGVALHRPD VYLLPPAREQ |
| | LNLRESATIT |
| 351/474 | CLVTGFSPAD VFVQWMQRGQ PLSPEKYVTS APMPEPQAPG |
| | RYFAHSILTV |
| 401/524 | SEEEWNTGET YTCVVAHEAL PNRVTERTVD KSTGKPTLYN |
| | VSLVMSDTAG |
| 451/574 | TCY |

Binding molecules, e.g., antibodies include, but are not limited to, polyclonal, monoclonal, human, humanized, or chimeric antibodies, single chain antibodies, epitope-binding fragments, e.g., Fab, Fab′ and F(ab′) 2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody, binds to an epitope via its antigen-binding domain, and that the binding entails some complementarity between the antigen-binding domain and the epitope. According to this definition, a binding molecule, e.g., an antibody is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

As used herein, the term “affinity” refers to a measure of the strength of the binding of an individual epitope with one or more antigen-binding domains, e.g., of an immunoglobulin molecule. See, e.g., Greenfield, E. A. (ed.), Antibodies: A Laboratory Manual, Second Edition, Cold Spring Harbor Laboratory Press, 2014. As used herein, the term “avidity” refers to the overall stability of the complex between a population of antigen-binding domains and an antigen. Avidity is related to both the affinity of individual antigen-binding domains in the population with specific epitopes and to the valencies of the immunoglobulins and the antigen. For example, the interaction between a bivalent monoclonal antibody and an antigen with a highly repeating epitope structure, such as a polymer, is considered to be one of high avidity. An interaction between a bivalent monoclonal antibody with a receptor present at a high density on a cell surface is also considered to be of high avidity.

Binding molecules, e.g., antibodies as disclosed herein, can also be described or specified in terms of their cross-reactivity. As used herein, the term “cross-reactivity” refers to the ability of a binding molecule, e.g., an antibody specific for one antigen, to react with a second antigen and is a measure of relatedness between two different antigenic substances. Thus, a binding molecule is cross-reactive if it binds to an epitope other than the one that induced its formation. The cross-reactive epitope generally contains many of the same complementary structural features as the inducing epitope, and in some cases, can actually fit better than the original.

“Antigen-binding antibody fragments” including single-chain antibodies or other antigen-binding domains can exist alone or in combination with one or more of the following: a hinge region, CH1, CH2, CH3, or CH4 domains, J chain, or secretory component. Also included are antigen-binding fragments that can include any combination of variable region(s) with one or more of a hinge region, CH1, CH2, CH3, or CH4 domains, a J chain, or a secretory component. Binding molecules, e.g., antibodies, can be from any animal origin including birds and mammals. The antibodies can be, e.g., human, murine, donkey, rabbit, goat, guinea pig, camel, llama, horse, or chicken antibodies. For example, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins, as described infra and, for example in, U.S. Pat. No. 5,939,598 by Kucherlapati et al. According to embodiments of the present disclosure, an IgM antibody as provided herein can include an antigen-binding fragment of an antibody, e.g., a scFv fragment, so long as the IgM antibody is able to form a multimer, e.g., a hexamer or a pentamer as used herein such a fragment comprises a “multimerizing fragment.”

As used herein, the term “heavy chain subunit” includes amino acid sequences derived from an immunoglobulin heavy chain of a binding molecule, e.g., an antibody. A binding molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody can include without limitation, in addition to a VH domain, a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, can include, in addition to a VH domain, a CH3 domain and a CH4 domain; or a CH3 domain, a CH4 domain, and a J chain. Further, a binding molecule, e.g., an antibody for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody or IgM-like antibody as provided herein comprises sufficient portions of an IgM heavy chain constant region to allow the IgM antibody or IgM-like antibody to form a multimer, e.g., a hexamer or a pentamer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Ck or CA) domain.

Binding molecules, e.g., antibodies, can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody that has antigen-binding domains for two or more different epitopes within a single binding molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which any portion of the antibody, e.g., a variable domain, constant region, and/or J chain is recombined with a heterologous antibody portion, and/or is altered by at least partial replacement of one or more amino acids. For example, entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of a different class, e.g., from an antibody of a different species. An engineered antibody in which one or more “donor” CDRs from a non-human antibody of known specificity are grafted into a human heavy or light chain framework region is referred to herein as a “humanized antibody.” In certain instances, not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial-and-error testing, to obtain a functional engineered or humanized antibody.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the “potency” of a binding molecule or a subunit thereof, e.g., an antibody or an individual binding domain of an antibody, is a measure of the molecule's activity expressed in terms of the amount required to produce a desired effect, either in vitro or in vivo. For example, a bivalent antibody specific for CD38 can have a specific potency for killing 50% of CD38-expressing cells in an in vitro assay, expressed as an EC50 measured either by mass/volume (e.g., in μg/ml) or as a molar concentration. Two different anti-CD38 antibodies can have vastly different potencies, meaning the EC50s of the molecules in a given assay differ significantly. Potency can also refer to the amount of a binding molecule required to achieve a desired result in a subject treated with the molecule, e.g., the amount of the molecule required to slow or stop progression of an autoimmune disorder or to reduce or remove signs or symptoms of an autoimmune disorder.

The use of a generic name of a biologic therapeutic is understood to include any and all biosimilars bearing the generic name, unless specifically stated otherwise. For example, use of the term “rituximab” would include e.g., rituximab, rituximab-pvvr, and rituximab-abbs.

Terms such as “treating” or “treatment” or “to treat” or “alleviating” or “to alleviate” refer to therapeutic measures that cure, slow down, lessen symptoms of, lessen the severity of symptoms of, and/or halt or slow the progression of an existing diagnosed pathologic condition or disorder. Terms such as “prevent,” “prevention,” “avoid,” “deterrence,” “prophylactic,” and the like refer to prophylactic or preventative measures that prevent the development of an undiagnosed targeted pathologic condition or disorder. Thus, “those in need of treatment” can include those already with the disorder.

By “subject” or “individual” or “animal” or “patient” or “mammal,” is meant any mammalian subject. In certain embodiments the subject is a subject for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

As used herein, the term “autoimmune disorder” refers to a disease or disorder wherein the subject's immune system pathogenically responds to one or more self-antigens as foreign antigens. Autoimmune disorders typically result in damage of otherwise healthy tissues and/or chronic inflammation.

### CD38 Target

CD38, also known as ADP-ribosyl cyclase/cyclic ADP-ribose hydrolase 1, is a single-pass type II membrane protein. It has several known functions, including as an ADP-ribosyl cyclase, a cyclic ADP-ribosyl (cADPr) hydrolase, and non-canonical receptor. The amino acid sequence of human CD38 is provided herein as SEQ ID NO: 14, and the cynomolgus monkey CD38 amino acid sequence is provided as SEQ ID NO: 15 (about 93% identical to human CD38). Human CD38 has a cytoplasmic domain from amino acids 1-21 of SEQ ID NO: 14, a transmembrane domain from amino acids 22-42 of SEQ ID NO: 14, and an extracellular domain from amino acids 43-300 of SEQ ID NO: 14.

CD38 is expressed in a variety of hematopoietic and non-hematopoietic cells at varying levels. CD38 is expressed on either the cell surface or in intracellular compartments. In immune cells, CD38 is expressed, e.g., in B cells (including B cell precursors, germinal center B cells, and plasma cells), macrophages, dendritic cells, natural killer (NK) cells, T cells, neutrophils, and monocytes. In immune cells, CD38 can possess many roles from modulating cell differentiation to modulating effector functions during inflammation. (Piedra-Quintero, Z. L., et al., Front. Immunol. 2020, 11:597959, doi: 10.3389/fimmu.2020.597959).

As described herein, the disclosure provides, among other things, a method of treating an autoimmune or inflammatory disorder in a subject in need thereof, the method comprising administering to the subject a multimeric bispecific antibody targeting CD38, in addition to possessing an antigen binding domain for CD3, i.e., a multimeric bispecific anti-CD38/anti-CD3 antibody.

### Methods of Treating an Autoimmune or Inflammatory Disorder

Providing new immunotherapies remains a challenge, in particular, designing an anti-CD38×CD3 immunotherapy, since CD38 is expressed on T cells, which are meant to be stimulated by a T cell engager to kill the target cell. While killing of CD38-expressing B cells is desired for treating autoimmune diseases characterized by production of autoantibodies, binding to CD38-expressing T cells can also cause T cells to undergo self-killing or “fratricide,” thereby reducing the effectiveness of the T cell engager therapy. Thus, providing an improved anti-CD38×CD3 immunotherapy remains a challenge for at least the reason provided above. The methods described herein are believed to address the above-noted challenge, among other things.

Provided herein is a method of treating an autoimmune or inflammatory disorder in a subject, e.g., an autoimmune or inflammatory disorder in a patient in need of treatment, the method comprising administering to the subject a multimeric bispecific anti-CD38/anti-CD3 antibody, such as a multimeric bispecific anti-CD38/anti-CD3 antibody disclosed herein. In some embodiments, the multimeric bispecific anti-CD38/anti-CD3 antibody is a multimeric antibody comprising five bivalent binding units and a modified J chain. Exemplary multimeric bispecific anti-CD38/anti-CD3 are provided in this disclosure and in PCT Publication No. WO 2023/150677A2, which is incorporated herein by reference in its entirety.

In some embodiments, the method comprises administering to the subject a therapeutically effective amount of a multimeric bispecific anti-CD38/anti-CD3 antibody as provided herein. By “therapeutically effective dose or amount” or “effective amount” is intended an amount of a multimeric bispecific anti-CD38/anti-CD3 antibody that when administered brings about a positive therapeutic response with respect to treatment of subject.

Effective doses of compositions for treatment of a disease or disorder vary depending upon many different factors, including means of administration, target site, physiological state of the subject, whether the subject is human or an animal, other medications administered, and whether administration is prophylactic or therapeutic. Usually, the subject is a human in need of treatment, but non-human mammals including transgenic mammals can also be treated.

In some embodiments, the autoimmune disorder is a disorder where B lineage cells produce pathogenic autoantibodies and/or contribute to chronic inflammation in the subject. In some embodiments, the autoimmune disorder is systemic lupus erythematosus (SLE), antiphospholipid syndrome (APS), idiopathic thrombocytopenia purpura (ITP), warm autoimmune hemolytic anemia (wAIHA), multiple sclerosis (MS), myasthenia gravis (MG), pemphigus vulgaris (PV), anti-neutrophil cytoplasmic autoantibody (ANCA) associated vasculitis (AAV), thyroid eye disease (TED), membranous glomerulonephritis (MGN), neuromyelitis optica (NMO), Guillain-Barré syndrome (GBS), chronic inflammatory demyelinating polyradiculoneuropathy (CIDP), IgA nephropathy, Goodpasture's syndrome, granulomatosis with polyangiitis (GPA), microscopic polyangiitis (MPA), Sjögren syndrome, Behcet's disease, alopecia areata, immunoglobulin G4-related disease (IgG4-RD), phospholipase A2 receptor-associated membranous nephropathy (PLA2R MN), myositis, type 1 diabetes, systemic sclerosis, or rheumatoid arthritis (RA). In some embodiments, the autoimmune disorder is systemic lupus erythematosus (SLE), antiphospholipid syndrome (APS), idiopathic thrombocytopenia purpura (ITP), warm autoimmune hemolytic anemia (wAIHA), multiple sclerosis (MS), myasthenia gravis (MG), pemphigus vulgaris (PV), anti-neutrophil cytoplasmic autoantibody (ANCA) associated vasculitis (AAV), thyroid eye disease (TED), membranous glomerulonephritis (MGN), Neuromyelitis optica (NMO), Guillain-Barré syndrome (GBS), chronic inflammatory demyelinating polyradiculoneuropathy (CIDP), IgA nephropathy, Goodpasture's syndrome, rheumatoid arthritis (RA), or scleroderma. In some more particular embodiments, the autoimmune disorder is systemic lupus erythematosus (SLE). In some further embodiments, the SLE comprises lupus nephritis (LN). In some additional embodiments, the autoimmune disorder is RA. In yet some other embodiments, the autoimmune disorder is MG.

Systemic lupus erythematosus (SLE) is systemic autoimmune disorder typically manifesting in immune system activation, the presence of autoantibodies against double-stranded DNA and inflammation of various organs. Lupus nephritis (LN) is the manifestation of SLE in the kidney. Methods of diagnosing SLE and LN are known, such as those methods described in Fava, A., et al., J Autoimmun. 96:1-13 (2019).

Antiphospholipid syndrome (APS) is an autoimmune disorder characterized by persistent antiphospholipid antibody that leads to thrombosis. Methods of diagnosing APS are known, such as those methods described in Garcia, D., et al., N Engl J Med 378:2010-2021 (2018).

Idiopathic thrombocytopenia purpura (ITP) is an autoimmune disorder where the immune system attacks platelets. ITP is typically characterized by a decrease in platelet counts and bleeding complications. Standard of care therapy is typically administration of a corticosteroid, although other therapy options are available. See Fillitz, M., et al., Memo—Magazine of European Medical Oncology 14:350-354 (2021).

Warm autoimmune hemolytic anemia (wAIHA) is an autoimmune disorder characterized by the destruction of red blood cells (RBCs) by “warm” antibodies. Warm antibodies are polyclonal immunoglobulins that maximally bind RBCs at 37° C. Methods of diagnosing wAIHA are known, such as those methods described in Jager, U., et al., 2019, Blood Reviews, doi: 10.1016/j.blre.2019.100648.

Multiple sclerosis (MS) is an autoimmune disorder that is characterized by demyelination and axonal transection, which can result in physical disability and cognitive impairment. Methods of diagnosing MS are known, such as those methods described in McGinley, M. P., et al., 2021, JAMA, 325 (8): 765-779.

Myasthenia gravis (MG) is characterized by autoantibodies against the neuromuscular junction. Methods of diagnosing MS are known, such as those methods described in Rousseff, 2021, J Clin Med, 10 (1736): 1-16.

Pemphigus vulgaris (PV) is characterized by autoantibodies against desmogleins 1 and 3, which typically result in skin lesions and lesions on the oral mucosa. Methods of diagnosing PV are known, such as those methods described in Popescu, et al., 2019, Experimental Therapeutic Medicine, 18 (6): 5056-5060.

Anti-neutrophil cytoplasmic autoantibody (ANCA) associated vasculitis (AAV) is characterized by autoantibodies against neutrophils and monocytes, which results in inflammation of blood vessels. Methods of diagnosing AAV are known, such as those methods described in Guchelaar, et al., Autoimmunity Reviews 20 (1): 102716 (2021).

Thyroid eye disease (TED) is believed to be caused by autoantibodies directed against receptors on the extraocular muscles (EOMs) and soft tissues of the orbit. Methods of diagnosing TED are known, such as those methods described in Manjandavida, F. P., et al., Kerala J Ophthalmol 32:10-26 (2020).

Membranous glomerulonephritis (MGN) is an autoimmune disorder characterized by deposits of immune complex at the glomerular membrane. Methods of diagnosing MGN are known, such as those methods described in Maifata, Biomedicines, 7 (86): 1-14 (2019).

Neuromyelitis optica (NMO) is an autoimmune disorder characterized by optic neuritis and myelitis. Methods of diagnosing NMO are known, such as those methods described in Fujihara, Curr Opin Neurol 32 (3): 385-394 (2019).

Guillain-Barre syndrome (GBS) is an autoimmune disorder of the peripheral nerves and is the most common cause of acute flaccid paralysis. Methods of diagnosing GBS are known, such as those methods described in Leonhard, S. E., et al., Nature Reviews Neurology 15:671-683 (2019).

Chronic inflammatory demyelinating polyradiculoneuropathy (CIDP) is an autoimmune disorder affecting the peripheral nerve and nerve root. Methods of diagnosing CIDP are known, such as those methods described in Stino, A. M., et al., Muscle Nerve 63 (2): 157-169 (2020).

IgA nephropathy is an autoimmune disorder characterized by the deposition of IgA in the glomerular mesangium. Methods of diagnosing CIDP are known, such as those methods described in Selvaskandan, H., et al., Clinical Experimental Nephrology 23:577-588 (2019).

Goodpasture's syndrome, also called anti-glomerular basement membrane (anti-GBM) or Goodpasture's disease, is an autoimmune disorder characterized by autoantibodies to type IV collagen antigens in the glomerular and alveolar basement membranes. Methods of diagnosing Goodpasture's syndrome are known, such as those methods described in Moiseev, S., et al. Autoimmunity Reviews, 19 (9): 102618 (2020).

Rheumatoid arthritis (RA) is an autoimmune disorder affecting joints. Methods of diagnosing RA are known, such as those methods described in Lin et al., Cells, 9 (880): 1-43 (2020).

Scleroderma is an autoimmune disorder characterized by production of autoantibodies, fibroblast dysfunction, small vessel vasculopathy. Methods of diagnosing scleroderma are known, such as those methods described in van den Hoogen et al., Arthritis and Rheumatism, 65 (11): 2737-2747 (2013).

Granulomatosis with polyangiitis (GPA) and Microscopic polyangiitis (MPA) are autoimmune disorders characterized by vasculitis. GPA was formerly known as Wegener's Granulomatosis. Methods of diagnosing GPA and MPA are known, such as those methods described in Lutalo et al., Journal of Autoimmunity 48-49:94-98 (2014) and Chung., S. A. and P. Seo, Rheum. Dis. Clin. North. Am. 36:545-548 (2010).

Sjögren syndrome is an autoimmune disorder characterized by lymphocytic infiltrate of the exocrine glands. Methods of diagnosing Sjögren syndrome are known, such as those methods described in Thorne et al., British Journal of Hospital Medicine, 78 (8): 438-442 (2017).

Behcet's disease is an autoimmune disorder characterized by blood vessel inflammation. Methods of diagnosing Behcet's disease are known, such as those methods described in Davatchi, Pathology Research International, 2012 (607921): 1-5 (2012).

Alopecia areata is an autoimmune disorder characterized by hair loss. Methods of diagnosing alopecia areata are known, such as those methods described in Finner, A. M., Dermatologic Therapy, 24:348-354 (2011).

Immunoglobulin G4-related disease (IgG4-RD) is an autoimmune disorder characterized by elevated serum IgG4 levels. Methods of diagnosing IgG4-RD are known, such as those methods described in Umehara, H., et al., Modern Rheumatology, 31 (3): 529-533 (2021).

Phospholipase A2 receptor-associated membranous nephropathy (PLA2R MN) is an autoimmune disorder characterized by anti-PLA2R autoantibodies. Methods of diagnosing PLA2R MN are known, such as those methods described in VanBeek, C., et al., Clinical Nephrology, 84:1-9 (2015).

Myositis is an autoimmune disorder characterized by muscle weakness and includes four subtypes: dermatomyositis (DM), polymyositis (PM), necrotizing myopathy (NM), and inclusion body myositis (IBM). Methods of diagnosing myositis are known, such as those methods described in Carstens et al., Clinical and Experimental Immunology, 175:425-438 (2014).

Type 1 diabetes is an autoimmune disorder characterized by pancreatic β-cell loss resulting in insulin deficiency. Methods of diagnosing type 1 diabetes are known, such as those methods described in Katsarou, A., et al., Nature Reviews Disease Primers, 3 (17016): 1-17 (2017).

Systemic sclerosis is an autoimmune disorder characterized by vascular abnormalities. Methods of diagnosing systemic are known, such as those methods described in Hudson, M., et al., Journal of Autoimmunity, 48-49:38-41 (2014).

In some embodiments of the method, the subject has not received any prior autoimmune disorder therapies. In some alternative embodiments, the subject has received a prior autoimmune disorder therapy. In some embodiments, the subject is resistant and/or refractory to the prior autoimmune disorder therapy. In some embodiments, the prior autoimmune disorder therapy comprises administration of a biologic (biological product). In some embodiments, the biologic is an anti-CD38 antibody, such as daratumumab or isatuximab. In some embodiments, the biologic is an anti-CD20 antibody, such as rituximab. In some embodiments, the prior autoimmune disorder therapy comprises an anti-B-lymphocyte stimulator (BlyS) antibody, e.g., belimumab. Belimumab (sold under the tradename BENLYSTAR) is US FDA approved for treating SLE. See BENLYSTA® Prescribing Information, revised July 2022.

In some embodiments, the prior autoimmune disorder therapy comprises administration of a TNF inhibitor such as infliximab, adalimumab, or etanercept. Infliximab (sold under the tradename REMICADE®) is US FDA approved for treatment of rheumatoid arthritis. See REMICADE® Prescribing Information, revised October 2021. Adalimumab (sold under the tradename HUMIRA®) is US FDA approved for treatment of rheumatoid arthritis. See HUMIRA® Prescribing Information, revised February 2021. Etanercept (sold under the tradename ENBREL®) is US FDA approved for treatment of rheumatoid arthritis. See ENBREL® Prescribing Information, revised December 2012.

In some embodiments, the administering of the multimeric bispecific anti-CD38/anti-CD3 antibody occurs at least 1 week, at least 2 weeks, at least 1 month, at least 2 months, at least 3 months, at least 6 months, at least 9 months, at least 1 year, or at least 2 years after the prior autoimmune disorder therapy was last administered. In some other embodiments, the administering of the multimeric bispecific anti-CD38/anti-CD3 antibody occurs less than 2 years, e.g., less than 1 year, less than 9 months, less than 6 months, less than 3 months, less than two months, or less than 1 month after the prior autoimmune disorder therapy was last administered. In some embodiments, the administering of the multimeric bispecific anti-CD38/anti-CD3 antibody occurs 1 week to 2 years after the prior autoimmune disorder therapy was last administered, such as 2 weeks to 1 year, 1 month to 1 year, 2 months to 1 year, 3 months to 1 year, 6 months to 1 year, or 9 months to 1 year.

In some embodiments of the method, the multimeric bispecific anti-CD38/anti-CD3 antibody kills a greater percentage of B cells than a reference anti-CD38 antibody. In some further embodiments, the multimeric bispecific anti-CD38/anti-CD3 antibody kills a greater percentage of B cells than a reference anti-CD38 antibody in the blood and/or secondary or tertiary lymphoid organs including but not limited to spleen, mesenteric lymph node, bone marrow, or combinations thereof. In some embodiments, the multimeric bispecific anti-CD38/anti-CD3 antibody kills a greater percentage of activated memory B cells than a reference anti-CD38 antibody. In some embodiments, the multimeric bispecific anti-CD38/anti-CD3 antibody kills a greater percentage of activated memory B cells than a reference anti-CD38 antibody in the blood and/or secondary or tertiary lymphoid organs including but not limited to spleen, mesenteric lymph node, bone marrow, or combinations thereof. In some embodiments, the reference anti-CD38 antibody is daratumumab or isatuximab.

In its simplest form, a preparation to be administered to a subject comprises a multimeric bispecific anti-CD38/anti-CD3 antibody as described herein administered in a conventional dosage form, wherein such preparation further comprises a pharmaceutical excipient, carrier or diluent as described elsewhere herein, such as in a composition as described herein.

A multimeric bispecific anti-CD38/anti-CD3 antibody as described herein can be administered by any suitable method, e.g., parenterally, intraventricularly, orally, by inhalation (e.g., as a spray or dry powder), topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, administration of the multimeric bispecific anti-CD38/anti-CD3 antibody comprises intravenous infusion. In some embodiments, administration of the multimeric bispecific anti-CD38/anti-CD3 antibody comprises subcutaneous administration.

Also provided herein is the use of a multimeric bispecific anti-CD38/anti-CD3 antibody as described herein in the manufacture of a medicament to treat an autoimmune or inflammatory disorder. Also provided herein is a multimeric bispecific anti-CD38/anti-CD3 antibody as described herein or a pharmaceutical composition comprising the multimeric bispecific anti-CD38/anti-CD3 antibody as described herein for use in a method disclosed herein.

### Pharmaceutical Compositions and Administration Methods

In certain embodiments, the multimeric bispecific anti-CD38/anti-CD3 antibody administered in the methods disclosed herein is comprised in a composition, e.g., a pharmaceutical composition. A composition as provided herein can further include a pharmaceutically acceptable carrier and/or excipient and can be formulated so as to be suitable for a desired mode of administration. See, e.g., the Handbook of Pharmaceutical Excipients, 9th edition, eds. Rowe, R. C., et al.

Methods of preparing a multimeric bispecific anti-CD38/anti-CD3 antibody employed in the methods as provided herein can be determined by a skilled person in view of this disclosure. A suitable pharmaceutical composition comprising the multimeric bispecific anti-CD38/anti-CD3 antibody can include a buffer (e.g., acetate, phosphate, or citrate buffer), and/or a surfactant (e.g., polysorbate), optionally a stabilizer (e.g., human albumin), etc.

A multimeric bispecific anti-CD38/anti-CD3 antibody as provided herein can be administered in a pharmaceutically effective amount for the treatment of a subject in need thereof, e.g., a subject having an autoimmune disorder. In this regard, it will be appreciated that the multimeric bispecific anti-CD38/anti-CD3 antibody can be formulated to facilitate administration and promote stability of the antibody. The “effective amount” of the provided multimeric bispecific anti-CD38/anti-CD3 antibody to treat an autoimmune disorder according to methods provided in this disclosure can be determined, e.g., by a healthcare provider upon assessing the severity and nature of the subject's autoimmune disorder and the particular bispecific anti-CD38/anti-CD3 antibody to be administered.

Pharmaceutical compositions accordingly can include a pharmaceutically acceptable, non-toxic, sterile aqueous carrier such as physiological saline which may optionally include non-toxic buffers, preservatives, and the like. Suitable formulations are described in Remington's Pharmaceutical Sciences (Mack Publishing Co.) 23rd ed. (2020).

Certain pharmaceutical compositions provided herein can be orally administered in an acceptable dosage form including, e.g., capsules, tablets, aqueous suspensions, or solutions.

Certain pharmaceutical compositions also can be administered by nasal aerosol or by inhalation (i.e., pulmonary delivery, e.g., in a form suitable for aerosol administration by nebulizer, a metered dose inhaler, or a dry powder inhaler). Suitable compositions can be prepared as solutions in saline, employing benzyl alcohol or other suitable preservatives, absorption promoters to enhance bioavailability, and/or other conventional solubilizing or dispersing agents. In some embodiments, the pharmaceutical composition is administered by nasal aerosol. In some embodiments, the pharmaceutical composition, such as a pharmaceutical composition for administration by nasal aerosol, comprises one or more of the following: a pH adjuster, such as HCl; a buffer; an emulsifier such as, for example, polysorbate or carbomer; a sugar or mono- or polyol, such as a monosaccharide (e.g., glucose, dextrose, or fructose), disaccharide (e.g., sucrose, lactose, or maltose), ribose, glycerine, sorbitol, xylitol, inositol, propylene glycol, galactose, mannose, xylose, rhamnose, glutaraldehyde, ethanol, mannitol, polyethylene glycol, glycerol, chitosal, phenylethyl alcohol; a preservative; cellulose, such as microcrystalline cellulose or carboxymethylcellulose; or mixtures thereof.

In some embodiments, the pharmaceutical composition is administered by inhalation. In some embodiments, the pharmaceutical composition, such as a pharmaceutical composition for administration by inhalation, is in the form of a dry powder, e.g., suitable for administration via a dry powder inhaler, or is in the form of a liquid, e.g., suitable for administration via a nebulizer, such as an airjet-compressor nebulizer or a mesh-based nebulizer. In some embodiments, the pharmaceutical composition, such as a pharmaceutical composition for administration by inhalation, comprises one or more of the following: a sugar or mono- or polyol, such as lactose, trelose, mannitol, sorbitol; a buffer, such as histidine, proline, or arginine; saline; polysorbate; or mixtures thereof.

The amount of a multimeric bispecific anti-CD38/anti-CD3 antibody that is combined with carrier materials to provide a single dosage form will vary depending, e.g., upon the subject treated, the particular multimeric bispecific anti-CD38/anti-CD3 antibody to be administered, and the particular mode of administration. The composition can be administered as a single dose, as multiple doses, or over an established period of time as an infusion. Dosage regimens also can be adjusted to provide the optimum desired response (e.g., a therapeutic or prophylactic response).

In keeping with the scope of the present disclosure, a multimeric bispecific anti-CD38/anti-CD3 antibody as described herein can be administered to a subject in need of therapy in an amount sufficient to produce a therapeutic effect. A multimeric bispecific anti-CD38/anti-CD3 antibody as provided herein can be administered to the subject in a conventional dosage form prepared by combining the multimeric bispecific anti-CD38/anti-CD3 antibody described herein with one or more conventional pharmaceutically acceptable carrier or diluent according to known techniques. The form and character of the pharmaceutically acceptable carrier or diluent is typically dictated by the amount of active ingredient with which it is to be combined, the route of administration and other well-known variables.

### Multimeric Bispecific Anti-CD38/Anti-CD3 Antibodies

Antibodies that can multimerize, such as IgM and IgA antibodies, have emerged as promising drug candidates, e.g., in the fields of immuno-oncology and infectious diseases, allowing for improved specificity, improved avidity, and the ability to bind to multiple binding targets. See, e.g., U.S. Pat. Nos. 9,951,134, 9,938,347, 10,351,631, 10,400,038, 10,570,191, 10,604,559, 10,618,978, 10,689,449, 10,787,520, 10,899,835, 11,401,337, 11,555,075, and 11,639,389, U.S. Patent Application Publication Nos. US 2019-0338041, US 2022-0289856 and US 2020-0392239, and PCT Publication Nos. WO 2022/178047A1 and WO 2023/150677A2, the contents of which are incorporated herein by reference in their entireties.

Provided herein are methods that employ a multimeric bispecific anti-CD38/anti-CD3 antibody, which is a multimeric antibody comprising five bivalent binding units and a modified J chain. In some embodiments, each binding unit comprises two IgM heavy chains, each comprising a heavy chain variable region (VH) and an IgM constant region or multimerizing fragment or variant thereof, and two light chains, each comprising a light chain variable region (VL) and a light chain constant region. In certain embodiments, the VH is situated amino terminal to the heavy chain constant region. In some embodiments, the VL is situated amino terminal to the light chain constant region, e.g., a kappa or lambda constant region. In some embodiments, the VH and VL combine to form a CD38 binding domain. In some embodiments, the modified J chain comprises a single-chain variable region (scFv) that specifically binds to CD3.

In some embodiments, each IgM heavy chain comprises the amino acid sequence SEQ ID NO: 198, each light chain comprises the amino acid sequence SEQ ID NO: 199, and the modified J chain comprises the amino acid sequence SEQ ID NO: 175.

In some embodiments, the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 (defined by Kabat) collectively comprise, respectively, the amino acid sequences SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 201, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95. In some embodiments, the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71.

In some embodiments, the VH and VL comprising the CDRs recited above each comprise, respectively, an amino acid sequence that is at least 80%, 85%, 90%, 95%, or 100% identical to SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142. In some embodiments where the HCDR1, HCDR2, HCDR3, LCDR1, LCDR2, and LCDR3 comprise, respectively, the amino acid sequences SEQ ID NO: 65, SEQ ID NO: 66, SEQ ID NO: 67, SEQ ID NO: 69, SEQ ID NO: 70, and SEQ ID NO: 71, the VH and VL comprise, respectively, an amino acid sequence that is at least 80%, 85%, 90%, 95%, or 100% identical to SEQ ID NO: 128 and SEQ ID NO: 133. In certain embodiments the VH and VL comprise, respectively, the amino acid sequences SEQ ID NO: 128 and SEQ ID NO: 133. In certain embodiments, the IgM heavy chain comprises the amino acid sequence SEQ ID NO: 198 and the light chain comprises the amino acid sequence SEQ ID NO: 199.

In some embodiments, the modified J chain comprises a J chain or functional fragment or variant thereof indirectly or directly fused to an scFv fragment that specifically binds to CD3. In some embodiments, the scFv fragment that specifically binds to CD3 comprises a heavy chain variable region (VH) and a light chain variable region (VL), wherein the VH comprises VH complementarity-determining regions VHCDR1, VHCDR2, and VHCDR3 and the VL comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49. In some embodiments the VHCDR1, VHCDR2, VHCDR3, VLCDR1, VLCDR2, and VLCDR3 comprise, respectively, SEQ ID NO: 25, SEQ ID NO: 26, SEQ ID NO: 27, SEQ ID NO: 29, SEQ ID NO: 30, and SEQ ID NO: 31. In some embodiments the VH and VL of the scFv fragment comprising the CDRs recited above each comprise, respectively, an amino acid sequence that is at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; SEQ ID NO: 46 and SEQ ID NO: 48; or SEQ ID NO: 50 and SEQ ID NO: 52. In some embodiments, the scFv fragment comprises the VH and VL amino acid sequences SEQ ID NO: 24 and SEQ ID NO: 28, respectively. In some embodiments, the scFv fragment comprises the amino acid sequence of SEQ ID NO: 166, SEQ ID NO: 171, SEQ ID NO: 173, SEQ ID NO: 178, SEQ ID NO: 183, SEQ ID NO: 188, or SEQ ID NO: 193. In some embodiments the scFv fragment comprises the amino acid sequence of SEQ ID NO: 173. In some embodiments the modified J chain comprises the amino acid sequence SEQ ID NO: 175.

In some embodiments, the multimeric bispecific anti-CD38/anti-CD3 antibody is IgM B-2.

IgM B-2 is an IgM-based bispecific antibody with ten CD38 binding domains and one CD3 binding domain. IgM B-2 triggers a cytotoxic T-lymphocyte (CTL) response and complement-dependent cytotoxicity (CDC) against CD38-expressing cells. IgM B-2 has been shown to be active in preclinical models of multiple myeloma. See, e.g., PCT Publication No. WO 2023/150677A2 and Li et al., ASH ANNUAL MEETING & EXPOSITION, Dec. 10-13, 2022, Publication No. 2678.

IgM is the first immunoglobulin produced by B cells in response to stimulation by antigen. IgM is naturally present at around 1.5 mg/ml in serum with a half-life of about 5 days. IgM is typically a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (u) constant region of IgM additionally contains a fourth constant domain (CH4) and includes a C-terminal “tailpiece.” The human IgM constant region typically comprises the amino acid sequence SEQ ID NO: 1 (identical to, e.g., GenBank Accession Nos. pir∥S37768, CAA47708.1, and CAA47714.1, allele IGHM*03) or SEQ ID NO: 2 (identical to, e.g., GenBank Accession No. sp|P01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cu 4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Five IgM binding units can form a complex with an additional small polypeptide chain (the J chain), or a functional fragment, variant, or derivative thereof, to form a pentameric IgM antibody, as discussed elsewhere herein. The precursor form of the human J chain is presented as SEQ ID NO: 6. The signal peptide extends from amino acid 1 to about amino acid 22 of SEQ ID NO: 6, and the mature human J chain extends from about amino acid 23 to amino acid 159 of SEQ ID NO: 6. The mature human J chain includes the amino acid sequence SEQ ID NO: 7.

Exemplary variant and modified J chains are described herein. Without the J chain, an IgM antibody or IgM-like antibody typically assembles into a hexamer, comprising up to twelve antigen-binding domains. With a J chain, an IgM antibody or IgM-like antibody typically assembles into a pentamer, comprising up to ten antigen-binding domains, or more, if the J chain is a modified J chain comprising one or more heterologous polypeptides comprising an additional antigen-binding domain(s). The assembly of five or six IgM binding units into a pentameric or hexameric IgM antibody or IgM-like antibody is thought to involve the Cμ4 and u tailpiece domains. See, e.g., Braathen, R., et al., J. Biol. Chem. 277:42755-42762 (2002). Accordingly, a pentameric or hexameric IgM antibody typically includes at least the Cμ4 and u tailpiece domains. A “multimerizing fragment” of an IgM heavy chain constant region thus includes at least the Cμ4 and μ tailpiece domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, a multimeric bispecific anti-CD38/anti-CD3 antibody as described herein comprises a complete IgM heavy (u) chain constant domain, e.g., SEQ ID NO: 1 or SEQ ID NO: 2, or a variant, derivative, or analog thereof, e.g., as provided herein.

In certain embodiments, the two IgM heavy chain constant regions included in each binding unit are human heavy chain constant regions. In some embodiments, the heavy chains are glycosylated. In some embodiments, the heavy chains can be mutated to affect glycosylation.

### J Chains and Functional Fragments or Variants Thereof

The multimeric bispecific anti-CD38/anti-CD3 antibody employed in the methods provided herein comprises a modified J chain comprising a J chain or functional fragment or variant thereof associated with, e.g., directly or indirectly fused to an scFv fragment that specifically binds to CD3. In certain embodiments, the modified J chain of a multimeric bispecific anti-CD38/anti-CD3 antibody employed in the methods provided herein comprises a naturally occurring J chain, such as a mature human J chain sequence (e.g., SEQ ID NO: 7). Alternatively, in some embodiments, the modified J chain of a multimeric bispecific anti-CD38/anti-CD3 antibody employed in the methods provided herein can comprise a variant J chain, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., U.S. Pat. No. 10,899,835, which is incorporated herein by reference in its entirety. In some embodiments, the modified J chain of a multimeric bispecific anti-CD38/anti-CD3 antibody employed in the methods provided herein can comprise a functional fragment of a naturally occurring or variant J chain. As persons of ordinary skill in the art will recognize, “a functional fragment” or a “functional variant” in this context includes those fragments and variants that can associate with binding units to form a pentameric IgM antibody and/or can associate with certain immunoglobulin receptors, e.g., pIgR.

In certain embodiments, the modified J chain can be mutated or otherwise engineered to affect, e.g., enhance, the serum half-life of the multimeric bispecific anti-CD38/anti-CD3antibody. In certain embodiments the J chain can be mutated to affect glycosylation.

In certain embodiments, the modified J chain of the multimeric bispecific anti-CD38/anti-CD3 antibody employed in the methods provided herein comprises a functional variant J chain that includes one or more single amino acid substitutions, deletions, or insertions relative to a reference J chain identical to the variant J chain except for the one or more single amino acid substitutions, deletions, or insertions. For example, certain J chain amino acid substitutions, deletions, or insertions can result in a multimeric bispecific anti-CD38/anti-CD3 antibody exhibiting an increased serum half-life upon administration to a subject animal relative to a reference multimeric bispecific anti-CD38/anti-CD3 antibody that is identical except for the one or more single amino acid substitutions, deletions, or insertions in the variant J chain, and is administered using the same method to the same animal species. In certain embodiments the variant J chain can include one, two, three, or four single amino acid substitutions, deletions, or insertions relative to the reference J chain.

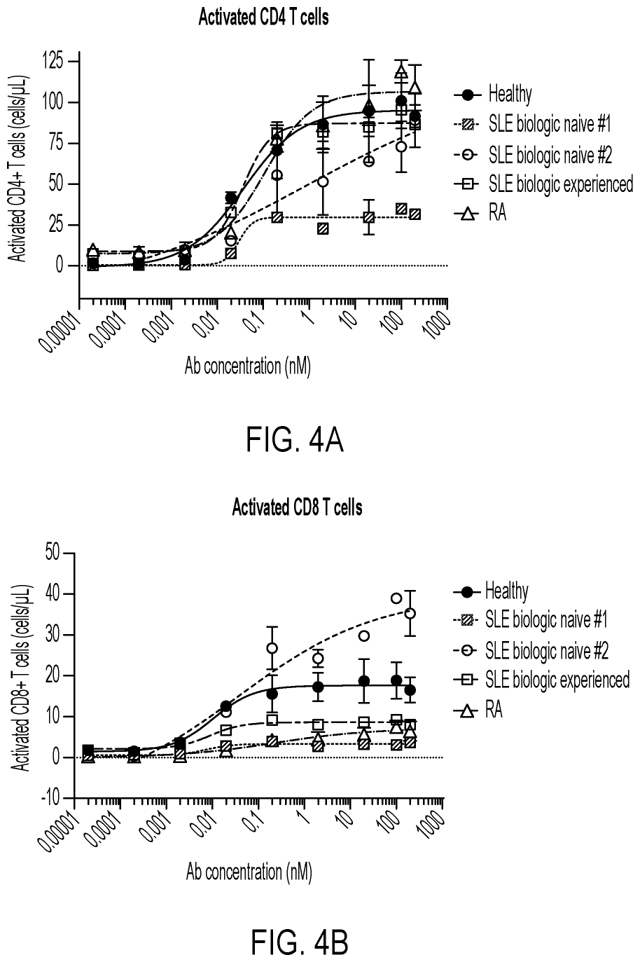

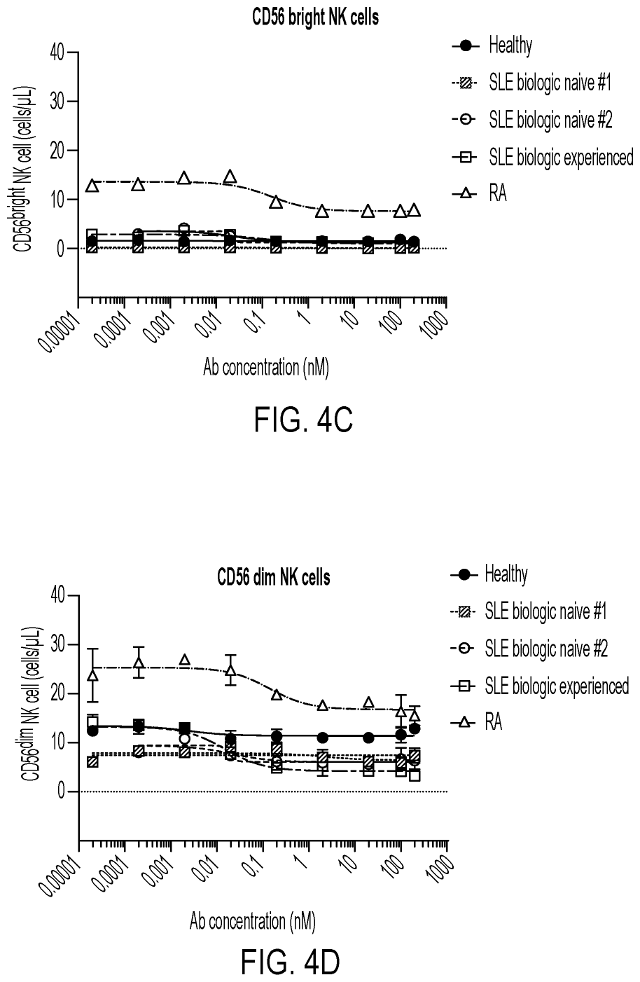

In certain embodiments, the modified J chain of a multimeric bispecific anti-CD38/anti-CD3 antibody employed in the methods provided herein comprises a variant J chain comprising an amino acid substitution at the amino acid position corresponding to amino acid Y102 of the mature wild-type human J chain (SEQ ID NO: 7). By “an amino acid corresponding to amino acid Y102 of the mature wild-type human J chain” is meant the amino acid in the sequence of the J chain, which is homologous to Y102 in the human J chain. For example, see U.S. Pat. No. 10,899,835, which is incorporated herein by reference in its entirety. The position corresponding to Y102 in SEQ ID NO: 7 is conserved in the J chain amino acid sequences of at least 43 other species. See FIG. 4 of U.S. Pat. No. 9,951,134, which is incorporated by reference herein. Certain mutations at the position corresponding to Y102 of SEQ ID NO: 7 can inhibit the binding of IgM pentamers comprising the variant J chain to certain immunoglobulin receptors, e.g., the human or murine Fcαμ receptor, the murine Fcμ receptor, and/or the human or murine polymeric Ig receptor (pIgR).

A multimeric bispecific anti-CD38/anti-CD3 antibody comprising a J chain mutation at the amino acid corresponding to Y102 of SEQ ID NO: 7, upon administration to a mammal, has a serum half-life that is improved over the serum half-life of a corresponding multimeric bispecific anti-CD38/anti-CD3 antibody that is identical except for the amino acid substitution, and which is administered to the same species in the same manner. In certain embodiments, the amino acid corresponding to Y102 of SEQ ID NO: 7 can be substituted with any amino acid. In certain embodiments, the amino acid corresponding to Y102 of SEQ ID NO: 7 can be substituted with alanine (A), serine(S) or arginine (R). In a particular embodiment, the amino acid corresponding to Y102 of SEQ ID NO: 7 can be substituted with alanine. In a particular embodiment, the J chain or functional fragment or variant thereof is a variant human J chain referred to herein as “J*,” and comprises the amino acid sequence SEQ ID NO: 8.

Wild-type J chains typically include one N-linked glycosylation site. In certain embodiments, a variant J chain or functional fragment thereof of a multimeric bispecific anti-CD38/anti-CD3 antibody described herein includes a mutation within the asparagine (N)-linked glycosylation motif N-X1-S/T, e.g., starting at the amino acid position corresponding to amino acid 49 (motif N6) of the mature human J chain (SEQ ID NO: 7) or J* (SEQ ID NO: 8), where Nis asparagine, X1 is any amino acid except proline, and S/T is serine or threonine, and where the mutation prevents glycosylation at that motif. As demonstrated in U.S. Pat. No. 10,899,835, J chain mutations preventing glycosylation at this site can result in a multimeric bispecific anti-CD38/anti-CD3 antibody exhibiting an increased serum half-life upon administration to a subject animal relative to a reference multimeric bispecific anti-CD38/anti-CD3 antibody that is identical except for the mutation or mutations preventing glycosylation in the variant J chain and is administered in the same way to the same animal species.

For example, in certain embodiments, the variant J chain or functional fragment thereof can include an amino acid substitution at the amino acid position corresponding to amino acid N49 or amino acid S51 of SEQ ID NO: 7 or SEQ ID NO: 8, provided that the amino acid corresponding to S51 is not substituted with threonine (T), or where the variant J chain comprises amino acid substitutions at the amino acid positions corresponding to both amino acids N49 and S51 of SEQ ID NO: 7 or SEQ ID NO: 8. In certain embodiments, the position corresponding to N49 of SEQ ID NO: 7 or SEQ ID NO: 8 is substituted with any amino acid, e.g., alanine (A), glycine (G), threonine (T), serine(S) or aspartic acid (D). In a particular embodiment, the position corresponding to N49 of SEQ ID NO: 7 or SEQ ID NO: 8 can be substituted with alanine (A). In another embodiment, the position corresponding to N49 of SEQ ID NO: 7 or SEQ ID NO: 8 can be substituted with aspartic acid (D). In some embodiments, the position corresponding to S51 of SEQ ID NO: 7 or SEQ ID NO: 8 is substituted with alanine (A) or glycine (G). In some embodiments, the position corresponding to S51 of SEQ ID NO: 7 or SEQ ID NO: 8 is substituted with alanine (A).

In certain embodiments, the modified J chain of the multimeric bispecific anti-CD38/anti-CD3 antibody employed in the methods provided herein comprises an scFv fragment that specifically binds to CD3 and can further comprise a heterologous moiety, e.g., a polypeptide, without interfering with the ability of the antibody to assemble and bind CD38. See, e.g., U.S. Pat. Nos. 9,951,134, 10,400,038, 10,618,978, and 11,639,389, each of which is incorporated herein by reference in its entirety.

Accordingly, in some embodiments, the anti-CD3 scFv fragment is fused to the J chain or fragment or variant thereof. In some embodiments, the scFv fragment is attached to the N-terminus of the J chain or functional fragment or variant thereof, the C-terminus of the J chain or functional fragment or variant thereof, or to both the N-terminus and C-terminus of the J chain or functional fragment or variant thereof. In certain embodiments, the scFv fragment is situated internally within the J chain or functional fragment or variant thereof. In some embodiments, the scFv fragment is situated at or near a glycosylation site in the J chain. In some embodiments, the scFv fragment is situated within about 10 amino acid residues from the C-terminus, or within about 10 amino acids from the N-terminus of the J chain. In certain embodiments, the scFv fragment and/or the heterologous polypeptide is situated in the mature human J chain of SEQ ID NO: 7 between cysteine residues 92 and 101 of SEQ ID NO: 7, or an equivalent location in a J chain sequence, e.g., a J chain variant or functional fragment of a J chain. In a further embodiment, the scFv fragment is situated in the mature human J chain of SEQ ID NO: 7 at or near a glycosylation site. In a further embodiment, the scFv fragment is situated in the mature human J chain of SEQ ID NO: 7 within about 10 amino acid residues from the C-terminus, or within about 10 amino acids from the N-terminus.

In certain embodiments, the anti-CD3 scFv fragment of the modified J chain of the multimeric bispecific anti-CD38/anti-CD3 antibody employed in the methods provided herein is fused in frame to the J chain. In certain embodiments, the anti-CD3 scFv fragment is fused to the J chain or functional fragment thereof via a peptide linker. Any suitable linker can be used, for example, the peptide linker can include at least 5 amino acids, at least ten amino acids, and least 20 amino acids, at least 30 amino acids or more, and so on. In certain embodiments, the peptide linker includes at least 5 amino acids, but no more than 25 amino acids. In certain embodiments, the peptide linker consists of 5 amino acids, 10 amino acids, 15 amino acids, 20 amino acids, or 25 amino acids. In certain embodiments, the peptide linker consists of GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), or GGGGSGGGGSGGGGSGGGGSGGGGS (SEQ ID NO: 13).

Additional heterologous moieties, e.g., heterologous polypeptides attached to a modified J chain for use in the methods provided herein, can include, without limitation, a binding moiety, e.g., an antibody or antigen-binding fragment thereof, e.g., a single chain Fv (scFv) molecule, a stabilizing peptide that can increase the half-life of the binding molecule, e.g., human serum albumin (HSA) or an HSA binding molecule, or a heterologous chemical moiety such as a polymer.

In certain embodiments, the scFv fragment that specifically binds to CD3 comprises a heavy chain variable region (VH) and a light chain variable region (VL), where the VH and VL collectively comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3, respectively, comprise the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49. In some embodiments, the VHCDR1, VHCDR2, VHCDR3, VLCDR1, VLCDR2, and VLCDR3 comprise, respectively, the amino acid sequences SEQ ID NO: 25, SEQ ID NO: 26, SEQ ID NO: 27, SEQ ID NO: 29, SEQ ID NO: 30, and SEQ ID NO: 31.

In some embodiments, the scFv fragment comprises an antibody VH and a VL comprising the CDRs recited above, where the VH and VL each comprise, respectively, amino acid sequences that are at least 80%, at least 85%, at least 90%, at least 95% or 100% identical to the amino acid sequences of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; SEQ ID NO: 46 and SEQ ID NO: 48; or SEQ ID NO: 50 and SEQ ID NO: 52. In some embodiments, the scFv fragment comprises the VH and VL amino acid sequences SEQ ID NO: 24 and SEQ ID NO: 28, respectively.

In some embodiments, the scFv fragment comprises the amino acid sequence of SEQ ID NO: 166, SEQ ID NO: 171, SEQ ID NO: 173, SEQ ID NO: 178, SEQ ID NO: 183, SEQ ID NO: 188, or SEQ ID NO: 193 In some embodiments the scFv fragment comprises the amino acid sequence of SEQ ID NO: 173.

In some embodiments, the modified J chain comprises the native human J chain amino acid sequence SEQ ID NO: 7 fused to an ScFv fragment that binds to CD3. In certain embodiments the modified J chain comprises the amino acid SEQ ID NO: 167, SEQ ID NO: 174, SEQ ID NO: 179, SEQ ID NO: 184, SEQ ID NO: 189, or SEQ ID NO: 194.

In some embodiments, the modified J chain of a multimeric bispecific anti-CD38/anti-CD3 antibody employed in the methods provided herein comprises a half-life extending moiety. In some embodiments, the half-life extending moiety is an albumin, such as a human serum albumin (HSA). In some embodiments, the modified J chain comprises the amino acid sequence SEQ ID NO: 169, SEQ ID NO: 176, SEQ ID NO: 181, SEQ ID NO: 186, SEQ ID NO: 191, or SEQ ID NO: 196. In some embodiments the modified J chain comprises a variant J chain sequence that extends the half-life of the multimeric bispecific anti-CD38/anti-CD3 antibody employed in the methods provided herein. In certain embodiments the modified J chain or functional fragment or variant thereof comprises an alanine (A) substitution at the amino acid position corresponding to amino acid Y102 of SEQ ID NO: 7. In certain embodiments the modified J chain or functional fragment or variant thereof comprises the amino acid sequence SEQ ID NO: 8. In certain embodiments the modified J chain comprises SEQ ID NO: 168, SEQ ID NO: 175, SEQ ID NO: 180, SEQ ID NO: 185, SEQ ID NO: 190, or SEQ ID NO: 195. In certain embodiments the modified J chain comprises the amino acid sequence SEQ ID NO: 175.

### Variant IgM Constant Regions

IgM heavy chain constant regions of a multimeric bispecific anti-CD38/anti-CD3 antibody employed in the methods provided herein can be engineered to confer certain desirable properties to the multimeric bispecific anti-CD38/anti-CD3 antibodies described herein. For example, in certain embodiments, IgM heavy chain constant regions can be engineered to confer enhanced serum half-life to multimeric bispecific anti-CD38/anti-CD3 antibodies as described herein. Exemplary IgM heavy chain constant region mutations that can enhance serum half-life of multimeric bispecific anti-CD38/anti-CD3 antibodies are disclosed in, e.g., U.S. Pat. No. 10,899,835, which is incorporated by reference herein in its entirety. For example, a variant IgM heavy chain constant region of a multimeric bispecific anti-CD38/anti-CD3 antibody described herein can include an amino acid substitution at a position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, such as e.g., alanine.

In certain embodiments, the IgM heavy chain constant regions of a multimeric bispecific anti-CD38/anti-CD3 antibody employed in the methods provided herein can be engineered to exhibit reduced complement-dependent cytotoxicity (CDC) activity to cells in the presence of complement, relative to a reference multimeric bispecific anti-CD38/anti-CD3 antibody with corresponding reference human IgM constant regions that are identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in U.S. Pat. No. 11,401,337, which is incorporated herein by reference in its entirety. Assays for measuring CDC are well known to those of ordinary skill in the art, and exemplary assays are described e.g., in U.S. Pat. No. 11,401,337.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid.

Human and certain non-human primate IgM constant regions typically include five (5) naturally occurring asparagine (N)-linked glycosylation motifs or sites. As used herein “an N-linked glycosylation motif” comprises or consists of the amino acid sequence N-X1-S/T, where Nis asparagine, X1 is any amino acid except proline (P), and S/T is serine(S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric bispecific anti-CD38/anti-CD3 antibody employed in the methods provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X1-S/T motifs can include an amino acid insertion, deletion, or substitution that prevents glycosylation at that motif. In certain embodiments, the IgM multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiments, the IgM constant region comprises one or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., PCT Publication No. WO 2021/041250, which is incorporated herein by reference in its entirety.

### Producing Multimeric Bispecific Anti-CD38/Anti-CD3 Antibodies

The multimeric bispecific anti-CD38/anti-CD3 antibodies used in methods disclosed herein can be produced from a host cell comprising one or more vectors encoding the polypeptide subunits of a multimeric bispecific anti-CD38/anti-CD3 antibody described herein using techniques known in the art. One vector can comprise nucleotide sequences encoding the heavy chain, light chain, and J chain. Alternatively, two or three vectors can encode any combination of the heavy chain, light chain, and J chain, such as one vector encoding the heavy chain and light chain, and a second vector encoding the J chain or three vectors, one encoding the heavy chain, one encoding the light chain, and one encoding the J chain.

### FURTHER EMBODIMENTS

Embodiment 1. A method of treating an autoimmune disorder comprising administering to a subject in need of treatment an effective amount of a multimeric bispecific anti-CD38/anti-CD3 antibody, which comprises five bivalent binding units and a modified J chain, wherein each binding unit comprises two IgM heavy chains, each comprising a heavy chain variable region (VH) and an IgM constant region or multimerizing fragment or variant thereof, and two light chains, each comprising a light chain variable region (VL) and a light chain constant region, wherein the VH comprises three immunoglobulin complementarity determining regions: HCDR1, HCDR2, and HCDR3, and the VL comprises three immunoglobulin complementarity determining regions: LCDR1, LCDR2, and LCDR3, wherein the HCDR1, HCDR2, HCDR3, LCDR1, LCDR2, and LCDR3 comprise, respectively, the amino acid sequences SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 201, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95, and wherein the modified J chain comprises a J chain or functional fragment or variant thereof indirectly or directly fused to an scFv molecule that specifically binds to CD3.

Embodiment 2. The method of embodiment 1, wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71.

Embodiment 3. The method of embodiment 1, wherein the VH and VL each comprise, respectively, an amino acid sequence that is at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142.

Embodiment 4. The method of embodiment 3, wherein the VH and VL each comprise, respectively, an amino acid sequence that is at least 80%, 85%, 90%, 95%, or 100% identical to the amino acid sequence of SEQ ID NO: 128 and SEQ ID NO: 133.

Embodiment 5. The method of embodiment 4, wherein the VH and VL comprise, respectively, the amino acid sequence of SEQ ID NO: 128 and SEQ ID NO: 133.

Embodiment 6. The method of any one of embodiments 1 to 5, wherein the IgM heavy chain constant region or multimerizing fragment or variant thereof comprises a Cμ4 region and a μ tailpiece region.

Embodiment 7. The method of embodiment 6, wherein the IgM heavy chain constant region or multimerizing fragment or variant thereof further comprises a Cμ1 region, a Cμ2 region, Cμ3 region, or any combination thereof.

Embodiment 8. The method of any one of embodiments 1 to 7, wherein the IgM heavy chain constant region or multimerizing fragment or variant thereof is a human IgM constant region.

Embodiment 9. The method of any one of embodiments 5 to 8, wherein the heavy chain comprises the amino acid sequence of SEQ ID NO: 198 and the light chain comprises the amino acid sequence of SEQ ID NO: 199.

Embodiment 10. A method of treating an autoimmune disorder comprising administering to a subject in need of treatment an effective amount of a multimeric bispecific anti-CD38/anti-CD3 antibody, which comprises five bivalent binding units and a modified J chain, wherein each binding unit comprises two IgM heavy chains, each comprising a heavy chain variable region (VH) and an IgM constant region or multimerizing fragment or variant thereof, and two light chains, each comprising a light chain variable region (VL) and a light chain constant region, wherein the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161, and wherein the modified J chain comprises a J chain or functional fragment or variant thereof indirectly or directly fused to an scFv molecule that specifically binds to CD3.

Embodiment 11. The method of any one of embodiments 1 to 10, wherein the scFv molecule that specifically binds to CD3 comprises a heavy chain variable region (VH) and a light chain variable region (VL), wherein the VH comprises VH complementarity-determining regions VHCDR1, VHCDR2, and VHCDR3 and the VL comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49.

Embodiment 12. The method of embodiment 11, wherein the VHCDR1, VHCDR2, VHCDR3, VLCDR1, VLCDR2, and VLCDR3 comprise, respectively, the amino acid sequences SEQ ID NO: 25, SEQ ID NO: 26, SEQ ID NO: 27, SEQ ID NO: 29, SEQ ID NO: 30, and SEQ ID NO: 31.

Embodiment 13. The method of embodiment 11, wherein the VH and VL of the scFv each comprise an amino acid sequence that is least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; SEQ ID NO: 46 and SEQ ID NO: 48; or SEQ ID NO: 50 and SEQ ID NO: 52, respectively.

Embodiment 14. The method of embodiment 13, wherein the scFv comprises the VH and VL amino acid sequences of SEQ ID NO: 24 and SEQ ID NO: 28, respectively.

Embodiment 15. The method of embodiment 13, wherein the scFv comprises the amino acid sequence of SEQ ID NO: 166, SEQ ID NO: 171, SEQ ID NO: 173, SEQ ID NO: 178, SEQ ID NO: 183, SEQ ID NO: 188, or SEQ ID NO: 193.

Embodiment 16. The method of embodiment 15, wherein the scFv comprises the amino acid sequence of SEQ ID NO: 173.

Embodiment 17. The method of any one of embodiments 1 to 16, wherein the modified J chain comprises the native human J chain amino acid sequence of SEQ ID NO:

7.

Embodiment 18. The method of embodiment 17, wherein the modified J chain comprises SEQ ID NO: 167, SEQ ID NO: 174, SEQ ID NO: 179, SEQ ID NO: 184, SEQ ID NO: 189, or SEQ ID NO: 194.

Embodiment 19. The method of embodiment 17, wherein the modified J chain further comprises human serum albumin indirectly or directly fused to the J chain or a functional fragment or variant thereof.

Embodiment 20. The method of embodiment 18, wherein the modified J chain comprises SEQ ID NO: 169, SEQ ID NO: 176, SEQ ID NO: 181, SEQ ID NO: 186, SEQ ID NO: 191, or SEQ ID NO: 196.

Embodiment 21. The method of any one of embodiments 1 to 16, wherein the modified J chain or functional fragment or variant thereof comprises an alanine (A) substitution at the amino acid position corresponding to amino acid Y102 of SEQ ID NO: 7.

Embodiment 22. The method of embodiment 21, wherein the modified J chain or functional fragment or variant thereof comprises the amino acid sequence of SEQ ID NO: 8.

Embodiment 23. The method of embodiment 22, wherein the modified J chain comprises SEQ ID NO: 168, SEQ ID NO: 175, SEQ ID NO: 180, SEQ ID NO: 185, SEQ ID NO: 190, or SEQ ID NO: 195.

Embodiment 24. The method of embodiment 23, wherein the modified J chain comprises SEQ ID NO: 175.

Embodiment 25. The method of embodiment 22, wherein the modified J chain further comprises human serum albumin indirectly or directly fused to the J chain or functional fragment or variant thereof.

Embodiment 26. The method of embodiment 23, wherein the modified J chain comprises SEQ ID NO: 170, SEQ ID NO: 177, SEQ ID NO: 182, SEQ ID NO: 187, SEQ ID NO: 192, or SEQ ID NO: 197.

Embodiment 27. The method of any one of embodiments 5 to 8, wherein the heavy chain comprises the amino acid sequence SEQ ID NO: 198, the light chain comprises the amino acid sequence SEQ ID NO: 199, and the modified J chain comprises the amino acid sequence SEQ ID NO: 175.

Embodiment 28. The method of any one of embodiments 1 to 27, wherein the autoimmune disorder is a disorder wherein CD38-expressing cells contribute to chronic inflammation in the subject.

Embodiment 29. The method of any one of embodiments 1 to 28, wherein the autoimmune disorder is systemic lupus erythematosus (SLE), antiphospholipid syndrome (APS), idiopathic thrombocytopenia purpura (ITP), warm autoimmune hemolytic anemia (wAIHA), multiple sclerosis (MS), myasthenia gravis (MG), pemphigus vulgaris (PV), anti-neutrophil cytoplasmic autoantibody (ANCA) associated vasculitis (AAV), thyroid eye disease (TED), membranous glomerulonephritis (MGN), Neuromyelitis optica (NMO), Guillain-Barré syndrome (GBS), chronic inflammatory demyelinating polyradiculoneuropathy (CIDP), IgA nephropathy, Goodpasture's syndrome, granulomatosis with polyangiitis (GPA), microscopic polyangiitis (MPA), Sjögren syndrome, Behcet's disease, alopecia areata, immunoglobulin G4-related disease (IgG4-RD), phospholipase A2 receptor-associated membranous nephropathy (PLA2R MN), myositis, type 1 diabetes, systemic sclerosis, or rheumatoid arthritis (RA).

Embodiment 30. The method of any one of embodiments 1 to 29, wherein the autoimmune disorder is SLE.

Embodiment 31. The method of embodiment 30, wherein the SLE comprises lupus nephritis (LN).

Embodiment 32. The method of any one of embodiments 1 to 29, wherein the autoimmune disorder is RA.

Embodiment 33. The method of any one of embodiments 1 to 29, wherein the autoimmune disorder is MG.

Embodiment 34. The method of any one of embodiments 1 to 33, wherein prior to the administering step, the subject had previously been treated with a biologic autoimmune disorder therapy.

Embodiment 35. The method of any one of embodiments 1 to 34, wherein prior to the administering step, the subject had previously been treated with an anti-CD20 antibody, an anti-B-lymphocyte stimulator (BlyS) antibody, or a TNF inhibitor.

Embodiment 36. The method of any one of embodiments 1 to 29, wherein the autoimmune disorder is SLE, and prior to the administering step, the subject had previously been treated with belimumab.

Embodiment 37. The method of any one of embodiments 1 to 29, wherein the autoimmune disorder is MS, and prior to the administering step, the subject had previously been treated with rituximab.

Embodiment 38. The method of any one of embodiments 1 to 29, wherein the autoimmune disorder is RA, and prior to the administering step, the subject had previously been treated with infliximab, adalimumab, or etanercept.

Embodiment 39. The method of any one of embodiments 1 to 38, wherein the administration of the multimeric bispecific anti-CD38/anti-CD3 antibody is via intravenous infusion.

Embodiment 40. The method of any one of embodiments 1 to 38, wherein the administration of the multimeric bispecific anti-CD38/anti-CD3 antibody is via subcutaneous injection.

Embodiment 41. The method of any one of embodiments 1 to 40 wherein the subject is a human.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All the references cited herein are incorporated herein by reference in their entireties.

The following examples are offered by way of illustration and not by way of limitation.

### EXAMPLES

### Example 1: Generation of CD38×CD3 Antibodies

Various anti-CD38×anti-CD3 IgM constructs were generated as described in PCT Publication No. WO 2023/150677A2 (which is incorporated herein in its entirety) using standard cloning protocols. In brief, heavy chain variable regions (VH) and light chain variable regions (VL) from anti-CD38 antibodies were cloned into an IgM format containing a modified J-chain comprising a CD3-binding single-chain variable fragment (scFv). The IgM antibodies that were generated are described in Table 2. The resulting constructs were expressed in Expi293 cells and purified according to the methods described in Keyt et al., Antibodies: 9:53, doi: 10.3390/antib9040053 (2020).

A CD38×CD3 IgG antibody comprising SEQ ID NOs: 162-164 was synthesized, expressed, and purified through a commercial vendor.

TABLE 2
Anti-CD38 × Anti-CD3 IgM Antibodies

| Antibody | | | J chain |
|---|---|---|---|
| Name | VH | VL | with scFv |
| IgM A-1 | SEQ ID NO: 56 | SEQ ID NO: 60 | SEQ ID NO: 200 |
| IgM B-1 | SEQ ID NO: 64 | SEQ ID NO: 68 | SEQ ID NO: 200 |
| IgM C-1 | SEQ ID NO: 72 | SEQ ID NO: 76 | SEQ ID NO: 200 |
| IgM E-1 | SEQ ID NO: 88 | SEQ ID NO: 92 | SEQ ID NO: 200 |
| IgM F-1 | SEQ ID NO: 96 | SEQ ID NO: 100 | SEQ ID NO: 200 |
| IgM H-1 | SEQ ID NO: 112 | SEQ ID NO: 116 | SEQ ID NO: 200 |
| IgM OKT10-2 | SEQ ID NO: 148 | SEQ ID NO: 151 | SEQ ID NO: 175 |
| IgM OKT10-4 | SEQ ID NO: 148 | SEQ ID NO: 151 | SEQ ID NO: 188 |
| IgM B-2 | SEQ ID NO: 128 | SEQ ID NO: 133 | SEQ ID NO: 175 |
| IgM B-3 | SEQ ID NO: 128 | SEQ ID NO: 133 | SEQ ID NO: 180 |
| IgM B-4 | SEQ ID NO: 128 | SEQ ID NO: 133 | SEQ ID NO: 188 |
| IgM B-5 | SEQ ID NO: 128 | SEQ ID NO: 133 | SEQ ID NO: 195 |
| IgM E-2 | SEQ ID NO: 136 | SEQ ID NO: 138 | SEQ ID NO: 175 |
| IgM E-3 | SEQ ID NO: 136 | SEQ ID NO: 138 | SEQ ID NO: 180 |
| IgM E-4 | SEQ ID NO: 136 | SEQ ID NO: 138 | SEQ ID NO: 188 |
| IgM E-5 | SEQ ID NO: 136 | SEQ ID NO: 138 | SEQ ID NO: 195 |

### Example 2: CD38×CD3 IgM Exhibits Reduced Fratricide Compared to CD38×CD3 IgG In Vitro

Designing anti-CD38×CD3 immunotherapies remains a challenge because CD38 is expressed on T cells, which are meant to be stimulated by a T cell engager to kill the target cell. While killing of CD38-expressing B cells is desired for treating autoimmune diseases characterized by production of autoantibodies, binding to CD38-expressing T cells can cause T cells to undergo self-killing or “fratricide,” reducing the effectiveness of the T cell engager therapy. To investigate the extent of fratricide from treatment with the CD38×CD3 IgM antibody, IgM B-2, the following experiments were performed.

T cell fratricide potential was tested in vitro using activated/expanded CD3+ pan T cells that have higher CD38 expression levels. Naïve T cells were activated and expanded using a T cell activation/expansion kit from Miltenyi Biotec (Cat #130-091-441). Activated T cells were then treated in vitro with 5 nM CD38×CD3 IgG or IgM B-2. T cell fratricide was evaluated at 72 h by flow cytometry analysis to quantitate live CD4+ and CD8+ T cells. The number of live cells per well was then normalized to the untreated control wells.

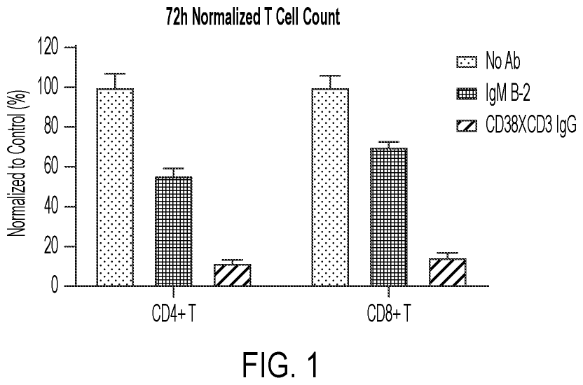

FIG. 1 shows that IgM B-2 favorably exhibits less T cell fratricide than CD38×CD3 IgG for both CD4+ and CD8+ T cells.

### Example 3: T Cell Dependent Cellular Cytotoxicity (TDCC)

2.5×10⁵ peripheral blood mononuclear cells (PBMCs) from various healthy and autoimmune disorder patient samples (1 healthy donor, 2 biologic naïve systemic lupus erythematosus (SLE) patients, 1 biologic experienced SLE patient, and 1 rheumatoid arthritis (RA) patient from Discovery Life Sciences and Sanguine Bio) were incubated in the presence of IgM B-2, at concentrations of 200 μg/mL, 100 μg/mL, and 20 μg/mL, followed by 10-fold serial dilutions from 2 to 0.00002 μg/mL in 200 μL of RPMI-1640 medium (Gibco, supplemented with heat-inactivated FBS, Glutamax, MEM amino acids solution, sodium pyruvate and penicillin-streptomycin) per well in a 96-well round bottom plate. After 72 hours at 37° C. in a 5% CO₂ incubator, the plates were centrifuged at 300×g at 4° C. The supernatants were recovered and tested for cytokine levels. The cells were then incubated for 15 minutes at room temperature with Fixable Viability Stain 780 (BD Biosciences). The plates were centrifuged at 300×g at 4° C. and washed twice in FACS stain buffer (phosphate buffered saline, heat-inactivated FBS, and EDTA) and then incubated with Fc Block (Thermo Fisher Scientific) for 30 min at 4° C. Following centrifugation at 300×g at 4° C., the plates were stained with the following BD Biosciences antibodies: R719 anti-human CD19, BV711 anti-human CD56, BUV737 anti-human CD69, PE anti-human CD38, APC anti-human CD4, V500 anti-human CD8, PerCPCy5.5 anti-human CD3, in Brilliant Stain buffer for 30 min at 4° C.

The plates were then centrifuged, washed, and the cells fixed in BD CYTOFIX™ fixation buffer for 15 minutes at 4° C. Cells were washed in FACS buffer and resuspended in 100 μL of FACS buffer with 10,000 COUNTBRIGHT™ Plus Absolute Counting Beads (Invitrogen) per well for acquisition on a BD FACSSYMPHONY™ A5 Cell Analyzer (BD Biosciences). The percent killing of CD19+CD38+B cells was quantified using the following equation:

$$100 \% - ( \% CD 19 + CD 38 + B cells in treatment sample \div \% CD 19 + CD 38 + B cells in untreated sample ) \times 100$$

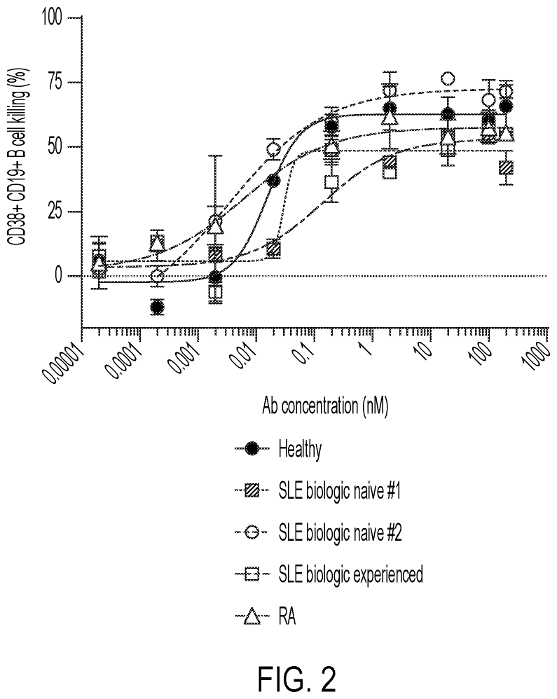

The results are shown in FIG. 2.

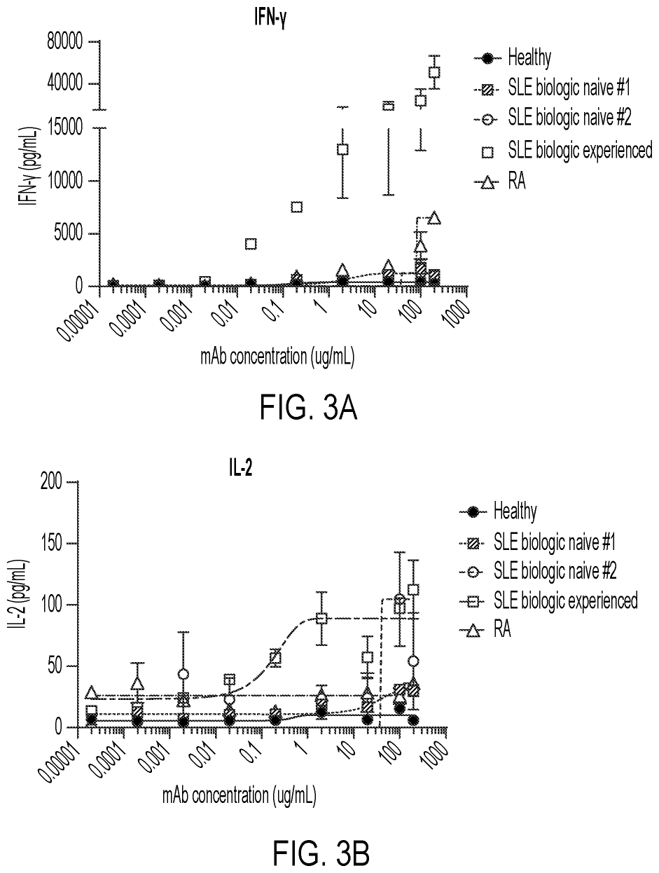

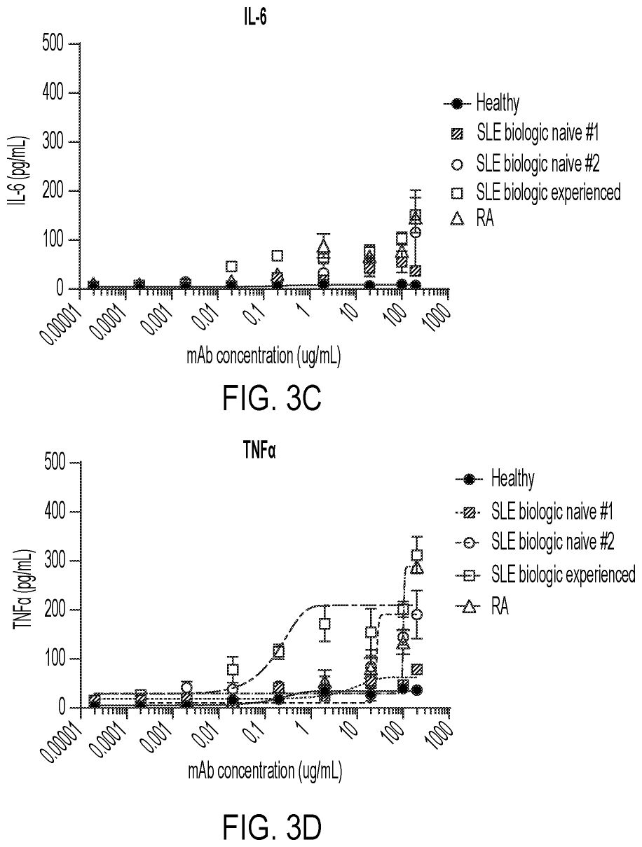

IFNγ, IL-2, IL-6, and TNFα release is shown in FIGS. 3A-3D, respectively, and were quantified in the supernatants of healthy (closed circle), SLE biologic naïve (closed square and open circle), SLE biologic experienced (open circle), and RA (closed triangle) PBMCs following 72 hour treatment with dilutions of IgM-B-2 as described above, using the V-PLEX Proinflammatory Panel 1 human kit from Meso Scale Discovery.

The number of activated CD4+ T cells (CD19-CD3+CD4+CD69+) and CD8+ T cells (CD19-CD3+CD8+CD69+) after treatment with IgM-B-2 as shown in FIGS. 4A and 4B were quantified using the COUNTBRIGHT™ Plus Absolute Counting Beads.

The number of peripheral NK cells (CD19-CD56bright) and hematic NK cells (CD19-CD56dim) after treatment with IgM B-2 are shown in FIGS. 4C and 4D and were quantified using the COUNTBRIGHT™ Plus Absolute Counting Beads.

### Example 4: T Cell Functional Assay

The impact of an anti-CD38×anti-CD3 IgM T cell engager (TCE) molecule (e.g., IgM B-2), as well as comparator molecules, on T cell activation and functional phenotypes in peripheral blood mononuclear cells (PBMCs) or bone marrow mononuclear cells (BMMCs) from healthy donors are assessed as follows. Fresh BMMCs were obtained from the University of Pennsylvania stem cell and xenograft core facility. Fresh PBMCs were isolated from whole blood, provided by BioIVT, with the SEPMATE™ PBMC Isolation Tubes (Stemcell Technologies) or frozen PBMCs were obtained from ALLCELLS™.

The cells (1.5×10⁵ cells/well) are plated in 100 μL of complete media (GIBCO® brand RPMI 1640 Medium, GlutaMAX Supplement, HEPES media with 10% (v/v) FBS, 1% (v/v) penicillin-streptomycin, 1% (v/v) MEM-NEAA, 1% (v/v) sodium pyruvate) in 96 well U bottom plates in duplicate per treatment.

Serial dilutions of IgM-B2, starting at a concentration 100 nM, are prepared. Serial dilutions of anti-CD38 IgG antibodies, e.g., daratumumab or isatuximab, starting at a concentration 1 μM, are likewise prepared. For a T cell activation positive control, an anti-human CD3 antibody, e.g., OKT3, available e.g., from Thermo Fisher Scientific, is prepared at a final assay concentration of 1 ng/mL. Each of the test articles and controls are prepared as a 2× concentrate.

Samples (100 μL) of the 2× antibody concentrates or of media are added to wells of the prepared 96-well plates and the samples are pipetted to mix. The plates are then incubated at 37° C. in a humidified incubator for 3 to 8 days.

On each collection day (starting on Day 3 post-antibody addition up to Day 8), plates to be harvested on that day are centrifuged at 300×g for 5 min. to pellet the cells. From each well, 100 μL of culture supernatant is collected and stored at −80° C. for subsequent cytokine analysis with the V-PLEX Proinflammatory Panel 1 human kit from Meso Scale Discovery.

Following recovery of the supernatants for cytokine analysis, 100 μL of complete media containing 2× cell stimulation and protein transport inhibitor cocktail (INVITROGEN®) is added to each well and pipetted to mix. The plates are then incubated for 4 hours in a 37° C., humidified incubator. At the end of the incubation, the plates are centrifuged at 300×g for 5 min. to pellet cells and the supernatants are removed.

To each well of the 96 well plates, 100 μL of Dulbecco's phosphate buffered saline (DPBS) is added and pipetted to mix. At this point, the duplicate wells are pooled together, each into a single well. The plates are then centrifuged at 300×g for 5 min. to pellet cells and supernatants removed.

To each sample well of the 96 well plates, 100 μL of a 1:200 dilution of live/dead stain (THERMOFISHER cat. #L34994 as described in Table 3 below) in DPBS is added and pipetted to mix. The plates are then incubated in the dark for 20 min. at room temperature. At the end of the incubation 100 μL of FACS buffer (DPBS with 1% (w/v) BSA and 5 mM EDTA) is added to each well. The plates are once again centrifuged at 300×g for 5 min. and supernatants removed.

A surface marker staining cocktail as per Table 3 is prepared by first adding Brilliant Stain buffer to the FACS buffer and then adding antibodies as indicated. 40.5 μL of cocktail is then added to each well and pipetted to mix. The plates are then incubated at 4° C. for 30 min. in the dark. 100 μL of FACS buffer is then added to each well. The plates are once again centrifuged at 300×g for 5 min. and supernatants removed.

TABLE 3
Flow cytometry staining panels post drug treatment

| | | | | | | Vol. (μL)/ |
|---|---|---|---|---|---|---|
| Marker | Fluorophore | Clone | Isotype | Vendor | Catalog # | sample |
| Live/Dead | Near IR | — | — | TF | L34994 | 1:200 Dil'n |

Surface staining reaction

| CD19 | APC | 4G7 | MG1 | BL | 392504 | 1.75 |
|---|---|---|---|---|---|---|
| CD3 | PerCP Cy5.5 | UCHT1 | MG1 | BL | 300430 | 1.75 |
| CD38 | FITC | Multi-Epitope | | BD | CYT-38F2-A | 2 |
| CD56 | BV711 | HCD56 | MG1 | BL | 318336 | 2.5 |
| CD25 | BV650 | M-A251 | MG1 | BD | 563719 | 1.75 |
| CD14 | BV605 | 63D3 | MG1 | BL | 367126 | 1.5 |
| CD8 | BV510 | SK1 | MG1 | BL | 344732 | 0.75 |
| CD69 | BUV737 | FN50 | MG1 | BD | 612817 | 1.5 |
| CD16 | BUV563 | 3G8 | MG1 | BD | 568289 | 1.5 |
| CD4 | BUV396 | SK3 | MG1 | BD | 563550 | 1.5 |

| | | | |
|---|---|---|---|
| Brilliant Stain Buffer Plus | BD | 566385 | 10 |

| | |
|---|---|
| FACS Buffer | 14.5 |

Intracellular staining reaction

| | | | | | | |
|---|---|---|---|---|---|---|
| TNFα | PE-Cy7 | MAb11 | MG1 | BL | 502930 | 2 |
| Ki67 | PE Dazzle 594 | Ki-67 | MG1 | BL | 350534 | 2 |
| Granzyme B | PE | QA16A02 | MG1 | BL | 372208 | 2 |
| Perforin | BV421 | dG9 | MG2 | BL | 308122 | 2 |

| | |
|---|---|
| 1 × Permeabilization buffer | 42 |

BD = Becton Dickinson;
BL = Biolegend;
MG1 = Mouse IgG1, κ;
MG2 = Mouse IgG2b, κ;
TF = ThermoFisher

Fixation and permeabilization buffers are prepared according to the manufacturer's instructions for the Foxp3/Transcription Factor Staining Buffer Set (INVITROGEN®). To each well of the 96 well plates, 200 μL of fixation/permeabilization buffer is added. The plates are then incubated in the dark at 4° C. for 45 min. The plates are then centrifuged at 300×g for 5 min. and supernatants removed. The plates are then washed two times with 200 μl of permeabilization buffer.

After washing, 50 μL of intracellular staining cocktail (see Table 3) is added to each well and pipetted to mix. The plates are then incubated in the dark at 4° C. for 30 min. An additional 150 μL of permeabilization buffer is then added to each well. The plates are then centrifuged at 300×g for 5 min. to pellet cells and supernatants removed. The plates are then washed two times with 150 μL of permeabilization buffer per well.

After removal of the second permeabilization buffer wash, each well is resuspended in 200 μL of FACS buffer. Data for each sample is acquired on a BD FACSYMPHONY™ A5 SE and analyzed in FLOWJO™. The gating hierarchy for acquired events is as follows: live→singlets→CD19⁻→CD56⁻→CD14→CD16⁻→CD3+→CD4×CD8. T cell activation is assessed by CD25 and CD69 upregulation on CD4 and CD8 populations. The percentage of CD4 and CD8 T cells expressing TNFα, perforin, granzyme B, IFNγ and Ki67 is quantified from the initial supernatants, as well as the gMFI of each marker.

### Example 5: TDCC Assay

The impact of an anti-CD38×anti-CD3 IgM T cell engager (TCE) molecule (e.g., IgM B-2), as well as comparator molecules, on T cell dependent cellular cytotoxicity (TDCC) in PBMCs or BMMCs from healthy donors is assessed as follows. Fresh BMMCs were obtained from the University of Pennsylvania stem cell and xenograft core facility. Fresh PBMCs were isolated from whole blood, provided by BioIVT, with the SEPMATE™ PBMC Isolation Tubes (Stemcell Technologies) or frozen PBMCs were obtained from ALLCELLS™.

The cells (2.5×10⁵ cells/well) are freshly isolated or thawed in complete media with 50 U/mL benzonase, are plated in 96 well U bottom plates in duplicate per treatment.

2-4×10⁶ cells per donor are reserved at this point to perform immuno-phenotyping and CD38 expression analysis at the time of assay setup (Day 0). These cells are stained according to the Day 0 flow cytometry staining in Table 4. Data for each sample is acquired on a BD FACSYMPHONY™ A5 SE and analyzed in FLOWJO™. CD38-HB7-PE expression (gMFI) is measured at baseline on cell populations described in Table 5.

TABLE 4
Flow cytometry staining panels

| | | | | | | Vol. (μL)/ |
|---|---|---|---|---|---|---|
| Marker | Fluorophore | Clone | Isotype | Vendor | Catalog # | sample |
| Live/Dead | Near IR | — | — | TF | L34994 | 1:200 Dil'n |

Day 0 immunophenotyping and CD38 expression analysis

| | | | | | | |
|---|---|---|---|---|---|---|
| CD19 | APC | 4G7 | MG1 | BL | 392504 | 1.75 |
| CD3 | PerCP Cy5.5 | UCHT1 | MG1 | BL | 300430 | 1.75 |
| CD45 | Spark Blue 574 | 2D1 | MG1 | BL | 368558 | 1.5 |
| CD138 | FITC | MI15 | MG1 | BL | 356508 | 2 |
| CD27 | PE-Cy7 | M-T271 | MG1 | BL | 356412 | 2 |
| CD319 | PE-Dazzle594 | 162.1 | MG2b | BL | 331812 | 2 |
| CD38 | PE | HB7 | MG1 | BD | 342371 | 20 |
| IgD | BV786 | IA6-2 | MG2a | BL | 348242 | 1.75 |
| CD45RA | BV750 | HI100 | MG2b | BL | 304166 | 1.75 |
| CD56 | BV711 | HCD56 | MG1 | BL | 318336 | 2.5 |
| CD25 | BV650 | M-A251 | MG1 | BD | 563719 | 1.75 |
| CD14 | BV605 | 63D3 | MG1 | BL | 367126 | 1.5 |
| CD8 | BV510 | SK1 | MG1 | BL | 344732 | 0.75 |
| CD20 | BV421 | S18015E | MG2a | BL | 375514 | 1.5 |
| CD24 | BUV805 | ML5 | MG2a | BD | 742010 | 1.5 |
| CD69 | BUV737 | FN50 | MG1 | BD | 612817 | 1.5 |
| CCR7 | BUV615 | 3D12 | RG2a | BD | 751103 | 1.5 |
| CD16 | BUV563 | 3G8 | MG1 | BD | 568289 | 1.5 |
| CD21 | BUV496 | B-ly4 | MG1 | BD | 750614 | 1.5 |
| CD4 | BUV396 | SK3 | MG1 | BD | 563550 | 1.5 |

| | | | |
|---|---|---|---|
| Brilliant Stain Buffer Plus | BD | 566385 | 10 |

Day 3 & day 5 TDCC assay

| CD19 | APC | 4G7 | MG1 | BL | 392504 | 1.75 |
|---|---|---|---|---|---|---|
| CD3 | PerCP Cy5.5 | UCHT1 | MG1 | BL | 300430 | 1.75 |
| CD45 | Spark Blue 574 | 2D1 | MG1 | BL | 368558 | 1.5 |
| CD38 | FITC | Multi-Epitope | | BD | CYT-38F2-A | 2 |
| CD27 | PE-Cy7 | M-T271 | MG1 | BL | 356412 | 2 |
| CD319 | PE-Dazzle594 | 162.1 | MG2b | BL | 331812 | 2 |
| CD138 | PE | MI15 | MG1 | BL | 356504 | 2 |
| IgD | BV786 | IA6-2 | MG2a | BL | 348242 | 1.75 |
| CD45RA | BV750 | HI100 | MG2b | BL | 304166 | 1.75 |
| CD56 | BV711 | HCD56 | MG1 | BL | 318336 | 2.5 |
| CD25 | BV650 | M-A251 | MG1 | BD | 563719 | 1.75 |
| CD14 | BV605 | 63D3 | MG1 | BL | 367126 | 1.5 |
| CD8 | BV510 | SK1 | MG1 | BL | 344732 | 0.75 |
| CD20 | BV421 | S18015E | MG2a | BL | 375514 | 1.5 |
| CD24 | BUV805 | ML5 | MG2a | BD | 742010 | 1.5 |
| CD69 | BUV737 | FN50 | MG1 | BD | 612817 | 1.5 |
| CCR7 | BUV615 | 3D12 | RG2a | BD | 751103 | 1.5 |
| CD16 | BUV563 | 3G8 | MG1 | BD | 568289 | 1.5 |
| CD21 | BUV496 | B-ly4 | MG1 | BD | 750614 | 1.5 |
| CD4 | BUV396 | SK3 | MG1 | BD | 563550 | 1.5 |

| | | | |
|---|---|---|---|
| Brilliant Stain Buffer Plus | BD | 566385 | 10 |

BD = Becton Dickinson;
BL = Biolegend;
MG1 = Mouse IgG1, κ;
MG2a = Mouse IgG2a, κ;
MG2b = Mouse IgG2b, κ;
RG2a = Rat IgG2a, κ;
TF = ThermoFisher

Serial dilutions of IgM-B2, starting at a concentration 100 nM, are prepared. Serial dilutions of anti-CD38 IgG antibodies, e.g., daratumumab or isatuximab, starting at a concentration of 1 μM, are likewise prepared. Each of the test articles and controls are prepared as a 2× concentrate.

To each well of the 96 well plates, 100 μL of the 2× antibody concentrates or media is added and pipetted to mix. The plates are then incubated at 37° C. in a humidified incubator for 3 or 5 days.

On each collection day (Day 3 post-antibody or Day 5 post-antibody) the plates centrifuged at 300×g for 5 min. to pellet cells. From each well, 175 μL of culture supernatant is collected and stored at −80° C. for subsequent cytokine analysis.

50 μL of PBS is added to each well and pipetted to mix. At this point, the non-adherent cells from duplicate wells are pooled together, each into a single well of a new 96 well U bottom plate.

Then, to remove adherent cells such as monocytes, 50 μL of pre-warmed TRYPLE™ cell detachment solution is added to each well of the original 96 well plates. The plates are then incubated for 5-7 minutes in a 37° C. humidified incubator. 50 μL of FACS buffer is then added to each well and pipetted to mix. Duplicate wells containing detached cells are pooled together into their corresponding well of the new set of plates to combine them with the non-adherent cell fraction from each sample. The plates are then spun at 300×g for 5 minutes and supernatants removed.

A surface marker staining cocktail as per Table 4 is prepared by first adding Brilliant Stain buffer to the FACS buffer and then adding antibodies as indicated. 43.5 μL of cocktail is then added to each well and pipetted to mix. The plates are then incubated at 4° C. for 30 min. in the dark. 150 μL of PBS is then added to each well. The plates are then centrifuged at 300×g for 5 min. and supernatants removed.

To each well of the 96 well plates, 100 μL of a 1:200 dilution of THERMOFISHER live/dead stain (cat. #L34994) in DPBS is added and pipetted to mix. The plates are then incubated in the dark for 20 min. at room temperature. At the end of the incubation, 100 μL of FACS buffer is added to each well. The plates are then centrifuged at 300×g for 5 min. and supernatants removed.

Cell pellets are then resuspended in 50 μL of fixation buffer and incubated in the dark for 15 minutes at room temperature. Following the incubation, 150 μL of FACS buffer is added to each well. The plates are then centrifuged at 300×g for 5 minutes and supernatants removed.

TABLE 5
Cell phenotyping strategy

| Population | Marker Phenotype (all | Population | Marker Phenotype (all |
|---|---|---|---|
| Name | CD45⁺, live, singlets) | Name | CD45⁺, live, singlets) |
| Plasma cells | CD3⁻CD19⁺CD20⁻CD27⁺ | Non-Classical | CD3⁻CD19⁻CD56⁻CD16⁺ |
| | CD138⁺CD319⁺ | Monocytes | CD14⁻ |
| Transitional B | CD3⁻CD19⁺IgD⁺CD27⁻ | CD4⁺ T cells | CD19⁻CD3⁺CD4⁺ |
| cells | CD24⁺FSC intermediate | | |
| Naïve B cells | CD3⁻CD19⁺IgD⁺CD27⁻SS | CD8⁺ T cells | CD19⁻CD3⁺CD8⁺ |
| | Clo | | |
| Double Neg. | CD3⁻CD19⁺IgD⁻CD27⁻ | CD4⁺CD38⁺ | CD19⁻CD3⁺CD4⁺CD38⁺ |
| (DN) B cells | | | |
| DN B cells- | CD3⁻CD19⁺IgD⁻CD27⁻ | CD8⁺CD38⁺ | CD19⁻CD3⁺CD8⁺CD38⁺ |
| Subset 1 | CD21⁻CD24⁺ | | |
| DN B cells- | CD3⁻CD19⁺IgD⁻CD27⁻ | Naïve CD4⁺ | CD19⁻CD3⁺CD4⁺CCR7⁺ |
| Subset 2 | CD21 + CD24 | T cells | CD45RA⁺ |
| Memory B | CD3⁻CD19⁺IgD⁻CD27⁺ | Naïve CD8 T | CD19⁻CD3⁺CD8⁺CCR7⁺ |
| cells | | cells | CD45RA⁺ |
| Unswitched B | CD3⁻CD19⁺IgD⁺CD27⁺ | CD4⁺ Eff. | CD19⁻CD3⁺CD4⁺CCR7⁻ |
| cells | | Memory | CD45RA⁻ |
| NK Cells | CD3⁻CD19⁻CD16⁺CD56 | CD4⁺ Central | CD19⁻CD3⁺CD4⁺CCR7⁺ |
| | intermediate | Memory | CD45RA⁻ |
| Non-classical | CD3⁻CD19⁻CD16⁻CD56⁻ | CD8⁺ Eff. | CD19⁻CD3⁺CD8⁺CCR7⁻ |
| NK Cells | hi | Memory | CD45RA⁻ |
| Classical | CD3⁻CD19⁻CD56⁻CD14⁺ | CD8⁺ Central | CD19⁻CD3⁺CD8⁺CCR7⁺ |
| Monocytes | CD16⁻ | Memory | CD45RA⁻ |
| Intermediate | CD3⁻CD19⁻CD56⁻CD16⁺ | | |
| Monocytes | CD14⁺ | | |

Each sample is resuspended in 200 μL of FACS buffer. Data for each sample is acquired on a BD FACSYMPHONY™ A5 SE and analyzed in FLOWJO™. CD38-HB7-PE expression (gMFI) is measured at baseline on cell populations described in Table 5. Percent killing for CD38+ cell populations is assessed on day 3 and day 5 using the following equation:

$$\% killing = ( 1 - ( cell count in treated sample / cell count in average of untreated samples ) ) \times 100.$$

T cell activation is assessed by CD25 and CD69 upregulation and NK cell activation by CD69 upregulation.

### Example 6: CD38×CD3 IgM Antibody IGM B-2 Kills Bone Marrow Derived Plasma Cells from Healthy Donors

Pathogenic autoantibodies are drivers of certain autoimmune diseases. Plasma cells and plasma blasts developing in the bone marrow express high levels of CD38. Depletion of these cell populations in autoimmune patients can lead to a reduction in circulating autoantibodies. See, e.g., Holzer, M T, et al., RMD Open 9 (4): e003604. doi: 10.1136/rmdopen-2023-003604 (2023).

The ability of IgM B-2 to kill plasma cells and plasmablasts in bone marrow cells from healthy donors was tested as follows. Freshly isolated bone marrow mononuclear cells (BMMCs) from 5 healthy donors were acquired from the University of Pennsylvania Stem Cell and Xenograft core facility. A T cell dependent cellular cytotoxicity (TDCC) assay was carried out as described in Example 5, using IgM B-2 as the test article, along with untreated control cells. BMMCs were incubated with the indicated dose range of IgM B-2 for 3 days. After the final recovery of cells, the abundance of CD138+CD319+ plasma cells (PCs) was measured by flow cytometry. Percent killing was then measured as follows:

$$\% killing = ( 1 - ( P C count in treated sample / P C count in untreated samples ) ) \times 100.$$

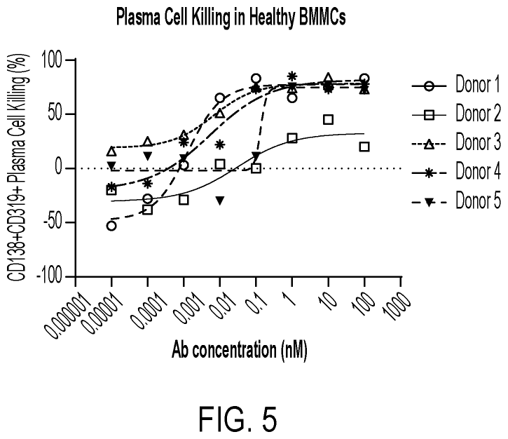

Data were plotted and modeled with a 4-parameter non-linear fit (FIG. 5). Four out of five donors demonstrated a maximum killing potential for IgM B-2 of 75%-85%.

TABLE 6
Sequences in the Disclosure

| | Nickname | |
|---|---|---|
| SEQ ID | (source) | Sequence |
| 1 | Human IgM | GSASAPTLFPLVSCENSPSDTSSVAVGCLAQDFLPDSITFSWKY |
| | Constant | KNNSDISSTRGFPSVLRGGKYAATSQVLLPSKDVMQGTDEHVVC |
| | region IMGT | KVQHPNGNKEKNVPLPVIAELPPKVSVFVPPRDGFFGNPRKSKL |
| | allele | ICQATGFSPRQIQVSWLREGKQVGSGVTTDQVQAEAKESGPTTY |
| | IGHM*03 | KVTSTLTIKESDWLSQSMFTCRVDHRGLTFQQNASSMCVPDQDT |
| | (GenBank: | AIRVFAIPPSFASIFLTKSTKLTCLVTDLTTYDSVTISWTRQNG |
| | pir\|S37768\|) | EAVKTHTNISESHPNATFSAVGEASICEDDWNSGERFTCTVTHT |
| | | DLPSPLKQTISRPKGVALHRPDVYLLPPAREQLNLRESATITCL |
| | | VTGFSPADVFVQWMQRGQPLSPEKYVTSAPMPEPQAPGRYFAHS |
| | | ILTVSEEEWNTGETYTCVVAHEALPNRVTERTVDKSTGKPTLYN |
| | | VSLVMSDTAGTCY |
| 2 | Human IgM | GSASAPTLFPLVSCENSPSDTSSVAVGCLAQDFLPDSITFSWKY |
| | Constant | KNNSDISSTRGFPSVLRGGKYAATSQVLLPSKDVMQGTDEHVVC |
| | region IMGT | KVQHPNGNKEKNVPLPVIAELPPKVSVFVPPRDGFFGNPRKSKL |
| | allele | ICQATGFSPRQIQVSWLREGKQVGSGVTTDQVQAEAKESGPTTY |
| | IGHM*04 | KVTSTLTIKESDWLGQSMFTCRVDHRGLTFQQNASSMCVPDQDT |
| | (GenBank: | AIRVFAIPPSFASIFLTKSTKLTCLVTDLTTYDSVTISWTRQNG |
| | sp\|P01871.4\|) | EAVKTHTNISESHPNATFSAVGEASICEDDWNSGERFTCTVTHT |
| | | DLPSPLKQTISRPKGVALHRPDVYLLPPAREQLNLRESATITCL |
| | | VTGFSPADVFVQWMQRGQPLSPEKYVTSAPMPEPQAPGRYFAHS |
| | | ILTVSEEEWNTGETYTCVVAHEALPNRVTERTVDKSTGKPTLYN |
| | | VSLVMSDTAGTCY |
| 3 | Human IgA1 | ASPTSPKVFPLSLCSTQPDGNVVIACLVQGFFPQEPLSVTWSES |
| | heavy chain | GQGVTARNFPPSQDASGDLYTTSSQLTLPATQCLAGKSVTCHVK |
| | constant | HYTNPSQDVTVPCPVPSTPPTPSPSTPPTPSPSCCHPRLSLHRP |
| | region, | ALEDLLLGSEANLTCTLTGLRDASGVTFTWTPSSGKSAVQGPPE |
| | e. g., amino | RDLCGCYSVSSVLPGCAEPWNHGKTFTCTAAYPESKTPLTATLS |
| | acids 144 to | KSGNTFRPEVHLLPPPSEELALNELVTLTCLARGFSPKDVLVRW |
| | 496 of | LQGSQELPREKYLTWASRQEPSQGTTTFAVTSILRVAAEDWKKG |
| | GenBank | DTFSCMVGHEALPLAFTQKTIDRLAGKPTHVNVSVVMAEVDGTC |
| | AIC59035.1 | Y |
| 4 | Human IgA2 | ASPTSPKVFPLSLDSTPQDGNVVVACLVQGFFPQEPLSVTWSES |
| | heavy chain | GQNVTARNFPPSQDASGDLYTTSSQLTLPATQCPDGKSVTCHVK |
| | constant | HYTNSSQDVTVPCRVPPPPPCCHPRLSLHRPALEDLLIGSEANL |
| | region, | TCTLTGLRDASGATFTWTPSSGKSAVQGPPERDLCGCYSVSSVL |
| | e.g., amino | PGCAQPWNHGETFTCTAAHPELKTPLTANITKSGNTFRPEVHLL |
| | acids 1 to | PPPSEELALNELVTLTCLARGFSPKDVLVRWLQGSQELPREKYL |
| | 340 of | TWASRQEPSQGTTTYAVTSILRVAAEDWKKGETFSCMVGHEALP |
| | GenBank | LAFTQKTIDRMAGKPTHINVSVVMAEADGTCY |
| | P01877.4 | |
| 5 | Precursor | MLLFVLTCLLAVFPAISTKSPIFGPEEVNSVEGNSVSITCYYPP |
| | Human | TSVNRHTRKYWCRQGARGGCITLISSEGYVSSKYAGRANLTNFP |
| | Secretory | ENGTFVVNIAQLSQDDSGRYKCGLGINSRGLSFDVSLEVSQGPG |
| | Component | LLNDTKVYTVDLGRTVTINCPFKTENAQKRKSLYKQIGLYPVLV |
| | | IDSSGYVNPNYTGRIRLDIQGTGQLLFSVVINQLRLSDAGQYLC |
| | | QAGDDSNSNKKNADLQVLKPEPELVYEDLRGSVTFHCALGPEVA |
| | | NVAKFLCRQSSGENCDVVVNTLGKRAPAFEGRILLNPQDKDGSF |
| | | SVVITGLRKEDAGRYLCGAHSDGQLQEGSPIQAWQLFVNEESTI |
| | | PRSPTVVKGVAGGSVAVLCPYNRKESKSIKYWCLWEGAQNGRCP |
| | | LLVDSEGWVKAQYEGRLSLLEEPGNGTFTVILNQLTSRDAGFYW |
| | | CLTNGDTLWRTTVEIKIIEGEPNLKVPGNVTAVLGETLKVPCHF |
| | | PCKFSSYEKYWCKWNNTGCQALPSQDEGPSKAFVNCDENSRLVS |
| | | LTLNLVTRADEGWYWCGVKQGHFYGETAAVYVAVEERKAAGSRD |
| | | VSLAKADAAPDEKVLDSGFREIENKAIQDPRLFAEEKAVADTRD |
| | | QADGSRASVDSGSSEEQGGSSRALVSTLVPLGLVLAVGAVAVGV |
| | | ARARHRKNVDRVSIRSYRTDISMSDFENSREFGANDNMGASSIT |
| | | QETSLGGKEEFVATTESTTETKEPKKAKRSSKEEAEMAYKDFLL |
| | | QSSTVAAEAQDGPQEA |
| 6 | Precursor | MKNHLLFWGVLAVFIKAVHVKAQEDERIVLVDNKCKCARITSRI |
| | Human J | IRSSEDPNEDIVERNIRIIVPLNNRENISDPTSPLRTRFVYHLS |
| | Chain | DLCKKCDPTEVELDNQIVTATQSNICDEDSATETCYTYDRNKCY |
| | | TAVVPLVYGGETKMVETALTPDACYPD |
| 7 | Mature Human | QEDERIVLVDNKCKCARITSRIIRSSEDPNEDIVERNIRIIVPL |
| | J Chain | NNRENISDPTSPLRTRFVYHLSDLCKKCDPTEVELDNQIVTATQ |
| | | SNICDEDSATETCYTYDRNKCYTAVVPLVYGGETKMVETALTPD |
| | | ACYPD |
| 8 | J Chain | QEDERIVLVDNKCKCARITSRIIRSSEDPNEDIVERNIRIIVPL |
| | Y102A | NNRENISDPTSPLRTRFVYHLSDLCKKCDPTEVELDNQIVTATQ |
| | mutation | SNICDEDSATETCATYDRNKCYTAVVPLVYGGETKMVETALTPD |
| | | ACYPD |
| 9 | “5” Peptide | GGGGS |
| | linker | |
| 10 | “10” Peptide | GGGGSGGGGS |
| | linker | |
| | (source) | |
| 11 | “15” Peptide | GGGGSGGGGSGGGGS |
| | linker | |
| 12 | “20” Peptide | GGGGSGGGGSGGGGSGGGGS |
| | linker | |
| 13 | “25” Peptide | GGGGSGGGGSGGGGSGGGGSGGGGS |
| | Linker | |
| 14 | human CD38 | MANCEFSPVSGDKPCCRLSRRAQLCLGVSILVLILVVVLAVVVP |
| | precursor | RWRQQWSGPGTTKRFPETVLARCVKYTEIHPEMRHVDCQSVWDA |
| | (GenBank: | FKGAFISKHPCNITEEDYQPLMKLGTQTVPCNKILLWSRIKDLA |
| | BAA18966.1) | HQFTQVQRDMFTLEDTLLGYLADDLTWCGEFNTSKINYQSCPDW |
| | | RKDCSNNPVSVFWKTVSRRFAEAACDVVHVMLNGSRSKIFDKNS |
| | | TFGSVEVHNLQPEKVQTLEAWVIHGGREDSRDLCQDPTIKELES |
| | | IISKRNIQFSCKNIYRPDKFLQCVKNPEDSSCTSEI |
| 15 | cyno CD38 | MANCEFSPVSGDKPCCRLSRRAQVCLGVCLLVLLILVVVVAVVL |
| | precursor | PRWRQQWSGSGTTSRFPETVLARCVKYTEVHPEMRHVDCQSVWD |
| | (GenBank: | AFKGAFISKYPCNITEEDYQPLVKLGTQTVPCNKTLLWSRIKDL |
| | AAT36330.1) | AHQFTQVQRDMFTLEDMLIGYLADDLTWCGEFNTFEINYQSCPD |
| | | WRKDCSNNPVSVFWKTVSRRFAETACGVVHVMLNGSRSKIFDKN |
| | | STFGSVEVHNLQPEKVQALEAWVIHGGREDSRDLCQDPTIKELE |
| | | SIISKRNIRFFCKNIYRPDKFLQCVKNPEDSSCLSGI |
| 162 | CD38 × CD3 IgG | EVQLVESGGGLVQPGGSLRLSCAASGFTFSTYAMNWVRQAPGKG |
| | scFv-Fc | LEWVGRIRSKANNYATYYADSVKGRFTISRDDSKNTLYLQMNSL |
| | chain | RAEDTAVYYCVRHGNFGDSYVSWFAYWGQGTLVTVSSGKPGSGK |
| | US20180305465 | PGSGKPGSGKPGSQAVVTQEPSLTVSPGGTVTLTCGSSTGAVTT |
| | | SNYANWVQQKPGKSPRGLIGGTNKRAPGVPARFSGSLLGGKAAL |
| | | TISGAQPEDEADYYCALWYSNHWFEGGGTKLTVLEPKSSDKTHT |
| | | CPPCPAPPVAGPSVFLFPPKPKDTLMISRTPEVTCVVVDVKHED |
| | | PEVKFNWYVDGVEVHNAKTKPREEQYNSTYRVVSVLIVLHQDWL |
| | | NGKEYKCKVSNKALPAPIEKTISKAKGQPREPQVYTLPPSREQM |
| | | TKNQVKLTCLVKGFYPSDIAVEWESNGQPENNYKTTPPVLDSDG |
| | | SFFLYSKLTVDKSRWQQGNVFSCSVMHEALHNHYTQKSLSLSPG |
| | | K |
| 163 | CD38 × CD3 IgG | DIVMTQSPSSLSASVGDRVTITCRASQNVDTWVAWYQQKPGQSP |
| | light chain | KALIYSASYRYSGVPDRFTGSGSGTDFTLTISSLQPEDFATYFC |
| | US20180305465 | QQYDSYPLTFGGGTKLEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| 164 | CD38 × CD3 IgG | EVQLVESGGGLVQPGGSLRLSCAASGFDFSRSWMNWVRQAPGKG |
| | heavy chain | LEWVSEINPDSSTINYATSVKGRFTISRDNSKNTLYLQMNSLRA |
| | US20180305465 | EDTAVYYCARYGNWFPYWGQGTLVTVSSASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSDTKVDKKVEPKSCD |
| | | KTHTCPPCPAPPVAGPSVFLFPPKPKDTLMISRTPEVTCVVVDV |
| | | KHEDPEVKFNWYVDGVEVHNAKTKPREEEYNSTYRVVSVLTVLH |
| | | QDWLNGKEYKCKVSNKALPAPIEKTISKAKGQPREPQVYTLPPS |
| | | REEMTKNQVSLTCDVSGFYPSDIAVEWESDGQPENNYKTTPPVL |
| | | DSDGSFFLYSKLTVDKSRWEQGDVFSCSVMHEALHNHYTQKSLS |
| | | LSPGK |
| 165 | HSA | DAHKSEVAHRFKDLGEENFKALVLIAFAQYLQQCPFEDHVKLVN |
| | | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKEGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFK |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGL |
| 166 | V scFv | QVQLVQSGAEVKKPGASVKVSCKASGYTFISYTMHWVRQAPGQG |
| | | LEWMGYINPRSGYTHYNQKLKDKATLTADKSASTAYMELSSLRS |
| | | EDTAVYYCARSAYYDYDGFAYWGQGTLVTVSSGGGGSGGGGSGG |
| | | GGSDIQMTQSPSSLSASVGDRVTITCSASSSVSYMNWYQQKPGK |
| | | APKRLIYDTSKLASGVPSRFSGSGSGTDFTITISSLQPEDFATY |
| | | YCQQWSSNPPTFGGGTKVEIK |
| 167 | V15J | QVQLVQSGAEVKKPGASVKVSCKASGYTFISYTMHWVRQAPGQG |
| | | LEWMGYINPRSGYTHYNQKLKDKATLTADKSASTAYMELSSLRS |
| | | EDTAVYYCARSAYYDYDGFAYWGQGTLVTVSSGGGGSGGGGSGG |
| | | GGSDIQMTQSPSSLSASVGDRVTITCSASSSVSYMNWYQQKPGK |
| | | APKRLIYDTSKLASGVPSRFSGSGSGTDFTITISSLQPEDFATY |
| | | YCQQWSSNPPTFGGGTKVEIKGGGGSGGGGSGGGGSQEDERIVL |
| | | VDNKCKCARITSRIIRSSEDPNEDIVERNIRIIVPLNNRENISD |
| | | PTSPLRTRFVYHLSDLCKKCDPTEVELDNQIVTATQSNICDEDS |
| | | ATETCYTYDRNKCYTAVVPLVYGGETKMVETALTPDACYPD |
| 168 | V15J* | QVQLVQSGAEVKKPGASVKVSCKASGYTFISYTMHWVRQAPGQG |
| | | LEWMGYINPRSGYTHYNQKLKDKATLTADKSASTAYMELSSLRS |
| | | EDTAVYYCARSAYYDYDGFAYWGQGTLVTVSSGGGGSGGGGSGG |
| | | GGSDIQMTQSPSSLSASVGDRVTITCSASSSVSYMNWYQQKPGK |
| | | APKRLIYDTSKLASGVPSRFSGSGSGTDFTLTISSLQPEDFATY |
| | | YCQQWSSNPPTFGGGTKVEIKGGGGSGGGGSGGGGSQEDERIVL |
| | | VDNKCKCARITSRIIRSSEDPNEDIVERNIRIIVPLNNRENISD |
| | | PTSPLRTRFVYHLSDLCKKCDPTEVELDNQIVTATQSNICDEDS |
| | | ATETCATYDRNKCYTAVVPLVYGGETKMVETALTPDACYPD |
| 169 | V15J15HSA | QVQLVQSGAEVKKPGASVKVSCKASGYTFISYTMHWVRQAPGQG |
| | | LEWMGYINPRSGYTHYNQKLKDKATLTADKSASTAYMELSSLRS |
| | | EDTAVYYCARSAYYDYDGFAYWGQGTLVTVSSGGGGSGGGGSGG |
| | | GGSDIQMTQSPSSLSASVGDRVTITCSASSSVSYMNWYQQKPGK |
| | | APKRLIYDTSKLASGVPSRFSGSGSGTDFTLTISSLQPEDFATY |
| | | YCQQWSSNPPTFGGGTKVEIKGGGGSGGGGSGGGGSQEDERIVL |
| | | VDNKCKCARITSRIIRSSEDPNEDIVERNIRIIVPLNNRENISD |
| | | PTSPLRTRFVYHLSDLCKKCDPTEVELDNQIVTATQSNICDEDS |
| | | ATETCYTYDRNKCYTAVVPLVYGGETKMVETALTPDACYPDGGG |
| | | GSGGGGSGGGGSDAHKSEVAHRFKDLGEENFKALVLIAFAQYLQ |
| | | QCPFEDHVKLVNEVTEFAKTCVADESAENCDKSLHTLEGDKLCT |
| | | VATLRETYGEMADCCAKQEPERNECFLQHKDDNPNLPRLVRPEV |
| | | DVMCTAFHDNEETFLKKYLYEIARRHPYFYAPELLFFAKRYKAA |
| | | FTECCQAADKAACLLPKLDELRDEGKASSAKQRLKCASLQKFGE |
| | | RAFKAWAVARLSQRFPKAEFAEVSKLVTDLTKVHTECCHGDLLE |
| | | CADDRADLAKYICENQDSISSKLKECCEKPLLEKSHCIAEVEND |
| | | EMPADLPSLAADFVESKDVCKNYAEAKDVFLGMELYEYARRHPD |
| | | YSVVLLLRLAKTYETTLEKCCAAADPHECYAKVFDEFKPLVEEP |
| | | QNLIKQNCELFKQLGEYKFQNALLVRYTKKVPQVSTPTLVEVSR |
| | | NLGKVGSKCCKHPEAKRMPCAEDYLSVVLNQLCVLHEKTPVSDR |
| | | VTKCCTESLVNRRPCFSALEVDETYVPKEFNAETFTFHADICTL |
| | | SEKERQIKKQTALVELVKHKPKATKEQLKAVMDDFAAFVEKCCK |
| | | ADDKETCFAEEGKKLVAASQAALGL |
| 170 | V15J*15HSA | QVQLVQSGAEVKKPGASVKVSCKASGYTFISYTMHWVRQAPGQG |
| | | LEWMGYINPRSGYTHYNQKLKDKATLTADKSASTAYMELSSLRS |
| | | EDTAVYYCARSAYYDYDGFAYWGQGTLVTVSSGGGGSGGGGSGG |
| | | GGSDIQMTQSPSSLSASVGDRVTITCSASSSVSYMNWYQQKPGK |
| | | APKRLIYDTSKLASGVPSRFSGSGSGTDFTITISSLQPEDEATY |
| | | YCQQWSSNPPTFGGGTKVEIKGGGGSGGGGSGGGGSQEDERIVL |
| | | VDNKCKCARITSRIIRSSEDPNEDIVERNIRIIVPLNNRENISD |
| | | PTSPLRTRFVYHLSDLCKKCDPTEVELDNQIVTATQSNICDEDS |
| | | ATETCATYDRNKCYTAVVPLVYGGETKMVETALTPDACYPD |
| | | GGGGSGGGGSGGGGSDAHKSEVAHRFKDLGEENFKALVLIAFAQ |
| | | YLQQCPFEDHVKLVNEVTEFAKTCVADESAENCDKSLHTLFGDK |
| | | LCTVATLRETYGEMADCCAKQEPERNECFLQHKDDNPNLPRLVR |
| | | PEVDVMCTAFHDNEETFLKKYLYEIARRHPYFYAPELLFFAKRY |
| | | KAAFTECCQAADKAACLLPKLDELRDEGKASSAKQRLKCASLQK |
| | | FGERAFKAWAVARLSQRFPKAEFAEVSKLVTDLTKVHTECCHGD |
| | | LLECADDRADLAKYICENQDSISSKLKECCEKPLLEKSHCIAEV |
| | | ENDEMPADLPSLAADFVESKDVCKNYAEAKDVFLGMELYEYARR |
| | | HPDYSVVLLLRLAKTYETTLEKCCAAADPHECYAKVFDEFKPLV |
| | | EEPQNLIKQNCELFKQLGEYKFQNALLVRYTKKVPQVSTPTLVE |
| | | VSRNLGKVGSKCCKHPEAKRMPCAEDYLSVVLNQLCVLHEKTPV |
| | | SDRVTKCCTESLVNRRPCFSALEVDETYVPKEFNAETFTFHADI |
| | | CTLSEKERQIKKQTALVELVKHKPKATKEQLKAVMDDFAAFVEK |
| | | CCKADDKETCFAEEGKKLVAASQAALGL |
| 171 | SP34 scFv | EVQLVESGGGLVQPKGSLKLSCAASGFTFNTYAMNWVRQAPGKG |
| | | LEWVARIRSKYNNYATYYADSVKDRFTISRDDSQSILYLQMNNL |
| | | KTEDTAMYYCVRHGNFGNSYVSWFAYWGQGTLVTVSSGGGGSGG |
| | | GGSGGGGSQAVVTQESALTTSPGETVTLTCRSSTGAVTTSNYAN |
| | | WVQEKPDHLFTGLIGGTNKRAPGVPARFSGSLIGDKAALTITGA |
| | | QTEDEAIYFCALWYSNLWVFGGGTKLTVL |
| 172 | SP3415J15HSA | EVQLVESGGGLVQPKGSLKLSCAASGFTFNTYAMNWVRQAPGKG |
| | | LEWVARIRSKYNNYATYYADSVKDRFTISRDDSQSILYLQMNNL |
| | | KTEDTAMYYCVRHGNFGNSYVSWFAYWGQGTLVTVSSGGGGSGG |
| | | GGSGGGGSQAVVTQESALTTSPGETVTLTCRSSTGAVTTSNYAN |
| | | WVQEKPDHLFTGLIGGINKRAPGVPARFSGSLIGDKAALTITGA |
| | | QTEDEAIYFCALWYSNLWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCYTYDRNKCYTAVVPLVYGGETKMVETALTPD |
| | | ACYPDGGGGSGGGGSGGGGSDAHKSEVAHRFKDLGEENFKALVL |
| | | IAFAQYLQQCPFEDHVKLVNEVTEFAKTCVADESAENCDKSLHT |
| | | LFGDKLCTVATLRETYGEMADCCAKQEPERNECFLQHKDDNPNL |
| | | PRLVRPEVDVMCTAFHDNEETFLKKYLYEIARRHPYFYAPELLF |
| | | FAKRYKAAFTECCQAADKAACLLPKLDELRDEGKASSAKQRLKC |
| | | ASLQKFGERAFKAWAVARLSQRFPKAEFAEVSKLVTDLTKVHTE |
| | | CCHGDLLECADDRADLAKYICENQDSISSKLKECCEKPLLEKSH |
| | | CIAEVENDEMPADLPSLAADFVESKDVCKNYAEAKDVFLGMFLY |
| | | EYARRHPDYSVVLLLRLAKTYETTLEKCCAAADPHECYAKVFDE |
| | | FKPLVEEPQNLIKQNCELFKQLGEYKFQNALLVRYTKKVPQVST |
| | | PTLVEVSRNLGKVGSKCCKHPEAKRMPCAEDYLSVVLNQLCVLH |
| | | EKTPVSDRVTKCCTESLVNRRPCFSALEVDETYVPKEENAETFT |
| | | FHADICTLSEKERQIKKQTALVELVKHKPKATKEQLKAVMDDFA |
| | | AFVEKCCKADDKETCFAEEGKKLVAASQAALGL |
| 173 | AA SCFV | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSNHWVFGGGTKLTVL |
| 174 | AA15J | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSNHWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCYTYDRNKCYTAVVPLVYGGETKMVETALTPD |
| | | ACYPD |
| 175 | AA15J* | EVQLLESGGGLVQPGGSLRLSCAASGFTEFTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSNHWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCATYDRNKCYTAVVPLVYGGETKMVETALTPD |
| | | ACYPD |
| 176 | AA15J15HSA | EVQLLESGGGLVQPGGSLRISCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSNHWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCYTYDRNKCYTAVVPLVYGGETKMVETALTPD |
| | | ACYPDGGGGSGGGGSGGGGSDAHKSEVAHRFKDLGEENFKALVL |
| | | IAFAQYLQQCPFEDHVKLVNEVTEFAKTCVADESAENCDKSLHT |
| | | LFGDKLCTVATLRETYGEMADCCAKQEPERNECFLQHKDDNPNL |
| | | PRLVRPEVDVMCTAFHDNEETFLKKYLYEIARRHPYFYAPELLF |
| | | FAKRYKAAFTECCQAADKAACLIPKLDELRDEGKASSAKQRLKC |
| | | ASLQKFGERAFKAWAVARLSQRFPKAEFAEVSKLVTDLTKVHTE |
| | | CCHGDLLECADDRADLAKYICENQDSISSKLKECCEKPLLEKSH |
| | | CIAEVENDEMPADLPSLAADFVESKDVCKNYAEAKDVFLGMFLY |
| | | EYARRHPDYSVVLLLRLAKTYETTLEKCCAAADPHECYAKVFDE |
| | | FKPLVEEPQNLIKQNCELFKQLGEYKFQNALLVRYTKKVPQVST |
| | | PTLVEVSRNLGKVGSKCCKHPEAKRMPCAEDYLSVVLNQLCVLH |
| | | EKTPVSDRVTKCCTESLVNRRPCFSALEVDETYVPKEFNAETFT |
| | | FHADICTLSEKERQIKKQTALVELVKHKPKATKEQLKAVMDDFA |
| | | AFVEKCCKADDKETCFAEEGKKLVAASQAALGL |
| 177 | AA15J*15HSA | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSNHWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCATYDRNKCYTAVVPLVYGGETKMVETALTPD |
| | | ACYPDGGGGSGGGGSGGGGSDAHKSEVAHRFKDLGEENFKALVL |
| | | IAFAQYLQQCPFEDHVKLVNEVTEFAKTCVADESAENCDKSLHT |
| | | LFGDKLCTVATLRETYGEMADCCAKQEPERNECFLQHKDDNPNL |
| | | PRLVRPEVDVMCTAFHDNEETFLKKYLYEIARRHPYFYAPELLF |
| | | FAKRYKAAFTECCQAADKAACLLPKLDELRDEGKASSAKQRLKC |
| | | ASLQKFGERAFKAWAVARLSQRFPKAEFAEVSKLVTDLTKVHTE |
| | | CCHGDLLECADDRADLAKYICENQDSISSKLKECCEKPLLEKSH |
| | | CIAEVENDEMPADLPSLAADFVESKDVCKNYAEAKDVFLGMFLY |
| | | EYARRHPDYSVVLLLRLAKTYETTLEKCCAAADPHECYAKVFDE |
| | | FKPLVEEPQNLIKQNCELFKQLGEYKFQNALLVRYTKKVPQVST |
| | | PTLVEVSRNLGKVGSKCCKHPEAKRMPCAEDYLSVVLNQLCVLH |
| | | EKTPVSDRVTKCCTESLVNRRPCFSALEVDETYVPKEFNAETFT |
| | | FHADICTLSEKERQIKKQTALVELVKHKPKATKEQLKAVMDDFA |
| | | AFVEKCCKADDKETCFAEEGKKLVAASQAALGL |
| 178 | AB SCFV | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSDLWVFGGGTKLTVL |
| 179 | AB15J | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSDLWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCYTYDRNKCYTAVVPLVYGGETKMVETALTPD |
| | | ACYPD |
| 180 | AB15J* | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSDLWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCATYDRNKCYTAVVPLVYGGETKMVETALTPD |
| | | ACYPD |
| 181 | AB15J15HSA | EVQLLESGGGLVQPGGSLRLSCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSDLWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCYTYDRNKCYTAVVPLVYGGETKMVETALTPD |
| | | ACYPDGGGGSGGGGSGGGGSDAHKSEVAHRFKDLGEENFKALVL |
| | | IAFAQYLQQCPFEDHVKLVNEVTEFAKTCVADESAENCDKSLHT |
| | | LFGDKLCTVATLRETYGEMADCCAKQEPERNECFLQHKDDNPNL |
| | | PRLVRPEVDVMCTAFHDNEETFLKKYLYEIARRHPYFYAPELLF |
| | | FAKRYKAAFTECCQAADKAACLLPKLDELRDEGKASSAKQRLKC |
| | | ASLQKFGERAFKAWAVARLSQRFPKAEFAEVSKLVTDLTKVHTE |
| | | CCHGDLLECADDRADLAKYICENQDSISSKLKECCEKPLLEKSH |
| | | CIAEVENDEMPADLPSLAADFVESKDVCKNYAEAKDVFLGMFLY |
| | | EYARRHPDYSVVLLLRLAKTYETTLEKCCAAADPHECYAKVFDE |
| | | FKPLVEEPQNLIKQNCELFKQLGEYKFQNALLVRYTKKVPQVST |
| | | PTLVEVSRNLGKVGSKCCKHPEAKRMPCAEDYLSVVLNQLCVLH |
| | | EKTPVSDRVTKCCTESLVNRRPCFSALEVDETYVPKEFNAETFT |
| | | FHADICTLSEKERQIKKQTALVELVKHKPKATKEQLKAVMDDFA |
| | | AFVEKCCKADDKETCFAEEGKKLVAASQAALGL |
| 182 | AB15J*15HSA | EVQLLESGGGLVQPGGSLRISCAASGFTFDTYAMNWVRQAPGKG |
| | | LEWVARIRSKYNNYATYYADSVKDRFTISRDDSKSTLYLQMESL |
| | | RAEDTAVYYCVRHANFGAGYVSWFAHWGQGTLVTVSSGGGGSGG |
| | | GGSGGGGSQTVVTQEPSLSVSPGGTVTLTCGSSTGAVTTSNYAN |
| | | WVQQTPGQAPRGLIGGTDKRAPGVPDRFSGSLLGDKAALTITGA |
| | | QAEDEADYYCALWYSDLWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCATYDRNKCYTAVVPLVYGGETKMVETALTPD |
| | | ACYPDGGGGSGGGGSGGGGSDAHKSEVAHRFKDLGEENFKALVL |
| | | IAFAQYLQQCPFEDHVKLVNEVTEFAKTCVADESAENCDKSLHT |
| | | LFGDKLCTVATLRETYGEMADCCAKQEPERNECFLQHKDDNPNL |
| | | PRLVRPEVDVMCTAFHDNEETFLKKYLYEIARRHPYFYAPELLF |
| | | FAKRYKAAFTECCQAADKAACLLPKLDELRDEGKASSAKQRLKC |
| | | ASLQKFGERAFKAWAVARLSQRFPKAEFAEVSKLVTDLTKVHTE |
| | | CCHGDLLECADDRADLAKYICENQDSISSKLKECCEKPLLEKSH |
| | | CIAEVENDEMPADLPSLAADFVESKDVCKNYAEAKDVFLGMFLY |
| | | EYARRHPDYSVVLLLRLAKTYETTLEKCCAAADPHECYAKVFDE |
| | | FKPLVEEPQNLIKQNCELFKQLGEYKFQNALLVRYTKKVPQVST |
| | | PTLVEVSRNLGKVGSKCCKHPEAKRMPCAEDYLSVVLNQLCVLH |
| | | EKTPVSDRVTKCCTESLVNRRPCFSALEVDETYVPKEFNAETFT |
| | | FHADICTLSEKERQIKKQTALVELVKHKPKATKEQLKAVMDDFA |
| | | AFVEKCCKADDKETCFAEEGKKLVAASQAALGL |
| 183 | AC scFv | QVQLVQSGAEVKKPGASVKVSCKASGFNIKDYYMHWVRQAPGQR |
| | | LEWMGWIDLENANTIYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCKQSYSRRTFGGGTKVEIK |
| 184 | AC15J | QVQLVQSGAEVKKPGASVKVSCKASGFNIKDYYMHWVRQAPGQR |
| | | LEWMGWIDLENANTIYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSIGERATINCKSSQSLLNARTGKNYLAWY |
| | | QQKPGQPPKLLIYWASTRESGVPDRFSGSGSGTDFTLTISSLQA |
| | | EDVAVYYCKQSYSRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCYTYDRNKCYTAVVPLVYGGETKMVETALTPDACY |
| | | PD |
| 185 | AC15J* | QVQLVQSGAEVKKPGASVKVSCKASGFNIKDYYMHWVRQAPGQR |
| | | LEWMGWIDLENANTIYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLINARTGKNYLAWY |
| | | QQKPGQPPKLLIYWASTRESGVPDRFSGSGSGTDFTLTISSLQA |
| | | EDVAVYYCKQSYSRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCATYDRNKCYTAVVPLVYGGETKMVETALTPDACY |
| | | PD |
| 186 | AC15J15HSA | QVQLVQSGAEVKKPGASVKVSCKASGFNIKDYYMHWVRQAPGQR |
| | | LEWMGWIDLENANTIYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCKQSYSRRTFGGGTKVEIKGGGGSGGGGSGGGGSDAH |
| | | KSEVAHRFKDLGEENFKALVLIAFAQYLQQCPFEDHVKLVNEVT |
| | | EFAKTCVADESAENCDKSLHTLFGDKLCTVATLRETYGEMADCC |
| | | AKQEPERNECFLQHKDDNPNLPRLVRPEVDVMCTAFHDNEETFL |
| | | KKYLYEIARRHPYFYAPELLFFAKRYKAAFTECCQAADKAACLL |
| | | PKLDELRDEGKASSAKQRLKCASLQKFGERAFKAWAVARLSQRF |
| | | PKAEFAEVSKLVTDLTKVHTECCHGDLLECADDRADLAKYICEN |
| | | QDSISSKIKECCEKPLLEKSHCIAEVENDEMPADLPSLAADFVE |
| | | SKDVCKNYAEAKDVFLGMFLYEYARRHPDYSVVLLLRLAKTYET |
| | | TLEKCCAAADPHECYAKVFDEFKPLVEEPQNLIKQNCELFKQLG |
| | | EYKFQNALLVRYTKKVPQVSTPTLVEVSRNLGKVGSKCCKHPEA |
| | | KRMPCAEDYLSVVLNQLCVLHEKTPVSDRVTKCCTESLVNRRPC |
| | | FSALEVDETYVPKEFNAETFTFHADICTLSEKERQIKKQTALVE |
| | | LVKHKPKATKEQLKAVMDDFAAFVEKCCKADDKETCFAEEGKKL |
| | | VAASQAALGL |
| 187 | AC15J*15HSA | QVQLVQSGAEVKKPGASVKVSCKASGFNIKDYYMHWVRQAPGQR |
| | | LEWMGWIDLENANTIYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCKQSYSRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCATYDRNKCYTAVVPLVYGGETKMVETALTPDACY |
| | | PDGGGGSGGGGSGGGGSDAHKSEVAHRFKDLGEENFKALVLIAF |
| | | AQYLQQCPFEDHVKLVNEVTEFAKTCVADESAENCDKSLHTLFG |
| | | DKLCTVATLRETYGEMADCCAKQEPERNECFLQHKDDNPNLPRL |
| | | VRPEVDVMCTAFHDNEETFLKKYLYEIARRHPYFYAPELLFFAK |
| | | RYKAAFTECCQAADKAACLLPKLDELRDEGKASSAKQRLKCASL |
| | | QKFGERAFKAWAVARLSQRFPKAEFAEVSKLVTDLTKVHTECCH |
| | | GDLLECADDRADLAKYICENQDSISSKLKECCEKPLLEKSHCIA |
| | | EVENDEMPADLPSLAADFVESKDVCKNYAEAKDVFLGMFLYEYA |
| | | RRHPDYSVVLLLRLAKTYETTLEKCCAAADPHECYAKVFDEFKP |
| | | LVEEPQNLIKQNCELFKQLGEYKFQNALLVRYTKKVPQVSTPTL |
| | | VEVSRNLGKVGSKCCKHPEAKRMPCAEDYLSVVLNQLCVLHEKT |
| | | PVSDRVTKCCTESLVNRRPCFSALEVDETYVPKEFNAETETFHA |
| | | DICTLSEKERQIKKQTALVELVKHKPKATKEQLKAVMDDFAAFV |
| | | EKCCKADDKETCFAEEGKKLVAASQAALGL |
| 188 | AD SCFV | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLINARTGKNYLAWY |
| | | QQKPGQPPKLLIYWASTRESGVPDRFSGSGSGTDFTLTISSLQA |
| | | EDVAVYYCKQSYFRRTFGGGTKVEIK |
| 189 | AD15J | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCKQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCYTYDRNKCYTAVVPLVYGGETKMVETALTPDACY |
| | | PD |
| 190 | AD15J* | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCKQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCATYDRNKCYTAVVPLVYGGETKMVETALTPDACY |
| | | PD |
| 191 | AD15J15HSA | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSIGERATINCKSSQSILNARTGKNYLAWY |
| | | QQKPGQPPKLLIYWASTRESGVPDRFSGSGSGTDFTLTISSLQA |
| | | EDVAVYYCKQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCYTYDRNKCYTAVVPLVYGGETKMVETALTPDACY |
| | | PDGGGGSGGGGSGGGGSDAHKSEVAHRFKDLGEENFKALVLIAF |
| | | AQYLQQCPFEDHVKLVNEVTEFAKTCVADESAENCDKSLHTLFG |
| | | DKLCTVATLRETYGEMADCCAKQEPERNECFLQHKDDNPNLPRL |
| | | VRPEVDVMCTAFHDNEETFLKKYLYEIARRHPYFYAPELLFFAK |
| | | RYKAAFTECCQAADKAACLLPKLDELRDEGKASSAKQRLKCASL |
| | | QKFGERAFKAWAVARLSQRFPKAEFAEVSKLVTDLTKVHTECCH |
| | | GDLLECADDRADLAKYICENQDSISSKLKECCEKPLLEKSHCIA |
| | | EVENDEMPADLPSLAADFVESKDVCKNYAEAKDVFLGMFLYEYA |
| | | RRHPDYSVVLLLRLAKTYETTLEKCCAAADPHECYAKVFDEFKP |
| | | LVEEPQNLIKQNCELFKQLGEYKFQNALLVRYTKKVPQVSTPTL |
| | | VEVSRNLGKVGSKCCKHPEAKRMPCAEDYLSVVLNQLCVLHEKT |
| | | PVSDRVTKCCTESLVNRRPCFSALEVDETYVPKEFNAETFTFHA |
| | | DICTLSEKERQIKKQTALVELVKHKPKATKEQLKAVMDDFAAFV |
| | | EKCCKADDKETCFAEEGKKLVAASQAALGL |
| 192 | AD15J*15HSA | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGRYFYDVWGQGTLVTVSSGGGGSGGGGSGGG |
| | | GSDIVMTQSPDSLAVSIGERATINCKSSQSLLNARTGKNYLAWY |
| | | QQKPGQPPKLLIYWASTRESGVPDRFSGSGSGTDFTLTISSLQA |
| | | EDVAVYYCKQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCATYDRNKCYTAVVPLVYGGETKMVETALTPDACY |
| | | PDGGGGSGGGGSGGGGSDAHKSEVAHRFKDLGEENFKALVLIAF |
| | | AQYLQQCPFEDHVKLVNEVTEFAKTCVADESAENCDKSLHTLFG |
| | | DKLCTVATLRETYGEMADCCAKQEPERNECFLQHKDDNPNLPRL |
| | | VRPEVDVMCTAFHDNEETFLKKYLYEIARRHPYFYAPELLFFAK |
| | | RYKAAFTECCQAADKAACLLPKLDELRDEGKASSAKQRLKCASL |
| | | QKFGERAFKAWAVARLSQRFPKAEFAEVSKLVTDLTKVHTECCH |
| | | GDLLECADDRADLAKYICENQDSISSKLKECCEKPLLEKSHCIA |
| | | EVENDEMPADLPSLAADFVESKDVCKNYAEAKDVFLGMFLYEYA |
| | | RRHPDYSVVLLLRLAKTYETTLEKCCAAADPHECYAKVFDEFKP |
| | | LVEEPQNLIKQNCELFKQLGEYKFQNALLVRYTKKVPQVSTPTL |
| | | VEVSRNLGKVGSKCCKHPEAKRMPCAEDYLSVVLNQLCVLHEKT |
| | | PVSDRVTKCCTESLVNRRPCFSALEVDETYVPKEFNAETFTFHA |
| | | DICTLSEKERQIKKQTALVELVKHKPKATKEQLKAVMDDFAAFV |
| | | EKCCKADDKETCFAEEGKKLVAASQAALGL |
| 193 | AE SCFV | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGQYFYDVWGQGTLVTVSSGGGGSGGGGSGGG |
| | | GSDIVMTQSPDSLAVSIGERATINCKSSQSLLNARTGKNYLAWY |
| | | QQKPGQPPKLLIYWASTRESGVPDRFSGSGSGTDFTLTISSLQA |
| | | EDVAVYYCTQSYFRRTFGGGTKVEIK |
| 194 | AE15J | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGQYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCTQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCYTYDRNKCYTAVVPLVYGGETKMVETALTPDACY |
| | | PD |
| 195 | AE15J* | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGQYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCTQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCATYDRNKCYTAVVPLVYGGETKMVETALTPDACY |
| | | PD |
| 196 | AE15J15HSA | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGQYFYDVWGQGTLVTVSSGGGGSGGGGSGGG |
| | | GSDIVMTQSPDSLAVSLGERATINCKSSQSLLNARTGKNYLAWY |
| | | QQKPGQPPKLLIYWASTRESGVPDRFSGSGSGTDFTLTISSLQA |
| | | EDVAVYYCTQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCYTYDRNKCYTAVVPLVYGGETKMVETALTPDACY |
| | | PDGGGGSGGGGSGGGGSDAHKSEVAHRFKDLGEENFKALVLIAF |
| | | AQYLQQCPFEDHVKLVNEVTEFAKTCVADESAENCDKSLHTLFG |
| | | DKLCTVATLRETYGEMADCCAKQEPERNECFLQHKDDNPNLPRL |
| | | VRPEVDVMCTAFHDNEETFLKKYLYEIARRHPYFYAPELLFFAK |
| | | RYKAAFTECCQAADKAACLLPKLDELRDEGKASSAKQRLKCASL |
| | | QKFGERAFKAWAVARLSQRFPKAEFAEVSKLVTDLTKVHTECCH |
| | | GDLLECADDRADLAKYICENQDSISSKLKECCEKPLLEKSHCIA |
| | | EVENDEMPADLPSLAADFVESKDVCKNYAEAKDVFLGMFLYEYA |
| | | RRHPDYSVVLLLRLAKTYETTLEKCCAAADPHECYAKVEDEFKP |
| | | LVEEPQNLIKQNCELFKQLGEYKFQNALLVRYTKKVPQVSTPTL |
| | | VEVSRNLGKVGSKCCKHPEAKRMPCAEDYLSVVLNQLCVLHEKT |
| | | PVSDRVTKCCTESLVNRRPCFSALEVDETYVPKEFNAETFTFHA |
| | | DICTLSEKERQIKKQTALVELVKHKPKATKEQLKAVMDDFAAFV |
| | | EKCCKADDKETCFAEEGKKLVAASQAALGL |
| 197 | AE15J*15HSA | QVQLVQSGAEVKKPGASVKVSCKASGFNIKDYYMHWVRQAPGQR |
| | | LEWIGWIDLENANTVYDAKFQGRVTITRDTSASTAYMELSSLRS |
| | | EDTAVYYCARDAYGQYFYDVWGQGTLVTVSSGGGGSGGGGSGGG |
| | | GSDIVMTQSPDSLAVSLGERATINCKSSQSLINARTGKNYLAWY |
| | | QQKPGQPPKLLIYWASTRESGVPDRFSGSGSGTDFTLTISSLQA |
| | | EDVAVYYCTQSYFRRTFGGGTKVEIKGGGGSGGGGSGGGGSQED |
| | | ERIVLVDNKCKCARITSRIIRSSEDPNEDIVERNIRIIVPLNNR |
| | | ENISDPTSPLRTRFVYHLSDLCKKCDPTEVELDNQIVTATQSNI |
| | | CDEDSATETCATYDRNKCYTAVVPLVYGGETKMVETALTPDACY |
| | | PDGGGGSGGGGSGGGGSDAHKSEVAHRFKDLGEENFKALVLIAF |
| | | AQYLQQCPFEDHVKLVNEVTEFAKTCVADESAENCDKSLHTLFG |
| | | DKLCTVATLRETYGEMADCCAKQEPERNECFLQHKDDNPNLPRL |
| | | VRPEVDVMCTAFHDNEETFLKKYLYEIARRHPYFYAPELLFFAK |
| | | RYKAAFTECCQAADKAACLLPKLDELRDEGKASSAKQRLKCASL |
| | | QKFGERAFKAWAVARLSQRFPKAEFAEVSKLVTDLTKVHTECCH |
| | | GDLLECADDRADLAKYICENQDSISSKLKECCEKPLLEKSHCIA |
| | | EVENDEMPADLPSLAADFVESKDVCKNYAEAKDVFLGMFLYEYA |
| | | RRHPDYSVVLLLRLAKTYETTLEKCCAAADPHECYAKVFDEFKP |
| | | LVEEPQNLIKQNCELFKQLGEYKFQNALLVRYTKKVPQVSTPTL |
| | | VEVSRNLGKVGSKCCKHPEAKRMPCAEDYLSVVLNQLCVLHEKT |
| | | PVSDRVTKCCTESLVNRRPCFSALEVDETYVPKEFNAETFTFHA |
| | | DICTLSEKERQIKKQTALVELVKHKPKATKEQLKAVMDDFAAFV |
| | | EKCCKADDKETCFAEEGKKLVAASQAALGL |
| 198 | IgM B-2 | QVQLVQSGAEVKKPGSSVKVSCKASGYTFTSYNMHWVRQAPGQG |
| | Heavy Chain | LEWMGYIYPGNGGTNYNQKFKGRVTITADESTSTAYMELSSLRS |
| | | EDTAVYYCARGASMITTGAWFAYWGQGTLVTVSSGSASAPTLEP |
| | | LVSCENSPSDTSSVAVGCLAQDFLPDSITFSWKYKNNSDISSTR |
| | | GFPSVLRGGKYAATSQVLLPSKDVMQGTDEHVVCKVQHPNGNKE |
| | | KNVPLPVIAELPPKVSVFVPPRDGFFGNPRKSKLICQATGFSPR |
| | | QIQVSWLREGKQVGSGVTTDQVQAEAKESGPTTYKVTSTLTIKE |
| | | SDWLSQSMFTCRVDHRGLTFQQNASSMCVPDQDTAIRVFAIPPS |
| | | FASIFLTKSTKLTCLVTDLTTYDSVTISWTRQNGEAVKTHTNIS |
| | | ESHPNATFSAVGEASICEDDWNSGERFTCTVTHTDLPSPLKQTI |
| | | SRPKGVALHRPDVYLLPPAREQLNLRESATITCLVTGFSPADVF |
| | | VQWMQRGQPLSPEKYVTSAPMPEPQAPGRYFAHSILTVSEEEWN |
| | | TGETYTCVVAHEALPNRVTERTVDKSTGKPTLYNVSLVMSDTAG |
| | | TCY |
| 199 | IgM B-2 | DIQMTQSPSSVSASVGDRVTITCRASESLDTYGNSFMYWYQQKP |
| | Light Chain | GKAPKLLIYLASSLESGVPSRFSGSGSGTDFTLTISSLQPEDFA |
| | | TYYCHQNHEDPWTFGQGTKVEIKRTVAAPSVFIFPPSDEQLKSG |
| | | TASVVCLLNNFYPREAKVQWKVDNALQSGNSQESVTEQDSKDST |
| | | YSLSSTLTLSKADYEKHKVYACEVTHQGLSSPVTKSFNRGEC |
| 200 | SJ* | EVQLVESGGGLVQPKGSLKLSCAASGFTFNTYAMNWVRQAPGKG |
| | | LEWVARIRSKYNNYATYYADSVKDRFTISRDDSQSILYLQMNNL |
| | | KTEDTAMYYCVRHGNFGNSYVSWFAYWGQGTLVTVSSGGGGSGG |
| | | GGSGGGGSQAVVTQESALTTSPGETVTLTCRSSTGAVTTSNYAN |
| | | WVQEKPDHLFTGLIGGTNKRAPGVPARFSGSLIGDKAALTITGA |
| | | QTEDEAIYFCALWYSNLWVFGGGTKLTVLGGGGSGGGGSGGGGS |
| | | QEDERIVLVDNKCKCARITSRIIRSSEDPNEDIVERNIRIIVPL |
| | | NNRENISDPTSPLRTRFVYHLSDLCKKCDPTEVELDNQIVTATQ |
| | | SNICDEDSATETCATYDRNKCYTAVVPLVYGGETKMVETALTPD |
| | | ACYPD |

TABLE 7
Sequences of exemplary CD3 binders
US5834597A
VH

| SEQ ID 16 | QVQLVQSGAEVKKPGASVKVSCKASGYTFISYTMHWVRQAPGQGLEWMGYINPRSGYTHYNQKLKDKATLTADK | | | | |
|---|---|---|---|---|---|
| | SASTAYMELSSLRSEDTAVYYCARSAYYDYDGFAYWGQGTLVTVSS | | | | |
| SEQ ID 17 | HCDR1 | | | | |
| | Sequence | SEQ ID | HCDR2 Sequence | SEQ ID | HCDR3 Sequence |
| | SYTMH | 18 | YINPRSGYTHYNQKLKD | 19 | SAYYDYDGFAY |

VL

| SEQ ID 20 | DIQMTQSPSSLSASVGDRVTITCSASSSVSYMNWYQQKPGKAPKRLIYDISKLASGVPSRFSGSGSGTDFTLTI | | | | |
|---|---|---|---|---|---|
| | SSLQPEDFATYYCQQWSSNPPTFGGGTKVEIK | | | | |
| | LCDR1 | | | | |
| | Sequence | SEQ ID | LCDR2 Sequence | SEQ ID | LCDR3 Sequence |
| SEQ ID 21 | SASSSVSYMN | 22 | DTSKLAS | 23 | QQWSSNPPT |

WO2018208864- xCD3 AA
VH

| SEQ ID 24 | EVQLLESGGGLVQPGGSLRLSCAASGFTFDTYAMNWVRQAPGKGLEWVARIRSKYNNYATYYADSVKDRFTISR | | | | |
|---|---|---|---|---|---|
| | DDSKSTLYLQMESLRAEDTAVYYCVRHANFGAGYVSWFAHWGQGTLVTVSS | | | | |
| | HCDR1 | | | | |
| | Sequence | SEQ ID | HCDR2 Sequence | SEQ ID | HCDR3 Sequence |
| SEQ ID 25 | TYAMN | 26 | RIRSKYNNYATYYADSVKD | 27 | HANFGAGYVSWFAH |

VL

| SEQ ID 28 | QTVVTQEPSLSVSPGGTVTLTCGSSTGAVTTSNYANWVQQTPGQAPRGLIGGTDKRAPGVPDRFSGSLLGDKAA | | | | |
|---|---|---|---|---|---|
| | LTITGAQAEDEADYYCALWYSNHWVFGGGTKLTVL | | | | |
| | LCDR1 | | | | |
| | Sequence | SEQ ID | LCDR2 Sequence | SEQ ID | LCDR3 Sequence |
| SEQ ID 29 | GSSTGAVTTSNYA | 30 | GTDKRAP | 31 | ALWYSNHWV |
| | N | | | | |

WO2018208864- xCD3 AB
VH

| SEQ ID 24 | EVQLLESGGGLVQPGGSLRLSCAASGFTFDTYAMNWVRQAPGKGLEWVARIRSKYNNYATYYADSVKDRFTISR | | | | |
|---|---|---|---|---|---|
| | DDSKSTLYLQMESLRAEDTAVYYCVRHANFGAGYVSWFAHWGQGTLVTVSS | | | | |
| | HCDR1 | | | | |
| | Sequence | SEQ ID | HCDR2 Sequence | SEQ ID | HCDR3 Sequence |
| SEQ ID 25 | TYAMN | 26 | RIRSKYNNYATYYADSVKD | 27 | HANFGAGYVSWFAH |

VL

| SEQ ID 32 | QTVVTQEPSLSVSPGGTVTLTCGSSTGAVTTSNYANWVQQTPGQAPRGLIGGTDKRAPGVPDRFSGSLLGDKAA | | | | |
|---|---|---|---|---|---|
| | LTITGAQAEDEADYYCALWYSDLWVFGGGTKLTVL | | | | |
| | LCDR1 | | | | |
| | Sequence | SEQ ID | LCDR2 Sequence | SEQ ID | LCDR3 Sequence |
| SEQ ID 29 | GSSTGAVTTSNYA | 30 | GTDKRAP | 33 | ALWYSDLWV |
| | N | | | | |

WO2018208864- xCD3 AC
VH

| SEQ ID 34 | QVQLVQSGAEVKKPGASVKVSCKASGFNIKDYYMHWVRQAPGQRLEWMGWIDLENANTIYDAKFQGRVTITRDT | | | | |
|---|---|---|---|---|---|
| | SASTAYMELSSLRSEDTAVYYCARDAYGRYFYDVWGQGTLVTVSS | | | | |
| | HCDR1 | | | | |
| | Sequence | SEQ ID | HCDR2 Sequence | SEQ ID | HCDR3 Sequence |
| SEQ ID 35 | DYYMH | 36 | WIDLENANTIYDAKFQG | 37 | DAYGRYFYDV |

VL

| SEQ ID 38 | DIVMTQSPDSLAVSLGERATINCKSSQSLLNARTGKNYLAWYQQKPGQPPKLLIYWASTRESGVPDRFSGSGSG | | | | |
|---|---|---|---|---|---|
| | TDFTLTISSLQAEDVAVYYCKQSYSRRTFGGGTKVEIK | | | | |
| | LCDR1 | | | | |
| | Sequence | SEQ ID | LCDR2 Sequence | SEQ ID | LCDR3 Sequence |
| SEQ ID 39 | KSSQSLLNARTGK | 40 | WASTRES | 41 | KQSYSRRT |
| | NYLA | | | | |

WO2018208864- xCD3 AD
VH

| SEQ ID 42 | QVQLVQSGAEVKKPGASVKVSCKASGFNIKDYYMHWVRQAPGQRLEWIGWIDLENANTVYDAKFQGRVTITRDT | | | | |
|---|---|---|---|---|---|
| | SASTAYMELSSLRSEDTAVYYCARDAYGRYFYDVWGQGTLVTVSS | | | | |
| | HCDR1 | | | | |
| | Sequence | SEQ ID | HCDR2 Sequence | SEQ ID | HCDR3 Sequence |
| SEQ ID 35 | DYYMH | 43 | WIDLENANTVYDAKEQG | 37 | DAYGRYFYDV |

VL

| SEQ ID 44 | DIVMTQSPDSLAVSIGERATINCKSSQSLLNARTGKNYLAWYQQKPGQPPKLLIYWASTRESGVPDRFSGSGSG | | | | |
|---|---|---|---|---|---|
| | TDFTLTISSLQAEDVAVYYCKQSYFRRTFGGGTKVEIK | | | | |
| | LCDR1 | | | | |
| | Sequence | SEQ ID | LCDR2 Sequence | SEQ ID | LCDR3 Sequence |
| SEQ ID 39 | KSSQSLLNARTGK | 40 | WASTRES | 45 | KQSYFRRT |
| | NYLA | | | | |

WO2018208864- xCD3 AE
VH

| SEQ ID 46 | QVQLVQSGAEVKKPGASVKVSCKASGFNIKDYYMHWVRQAPGQRLEWIGWIDLENANTVYDAKFQGRVTITRDT | | | | |
|---|---|---|---|---|---|
| | SASTAYMELSSLRSEDTAVYYCARDAYGQYFYDVWGQGTLVTVSS | | | | |
| | HCDR1 | | | | |
| | Sequence | SEQ ID | HCDR2 Sequence | SEQ ID | HCDR3 Sequence |
| SEQ ID 35 | DYYMH | 43 | WIDLENANTVYDAKFQG | 47 | DAYGQYFYDV |

VL

| SEQ ID 48 | DIVMTQSPDSLAVSLGERATINCKSSQSLLNARTGKNYLAWYQQKPGQPPKLLIYWASTRESGVPDRFSGSGSG | | | | |
|---|---|---|---|---|---|
| | TDFTLTISSLQAEDVAVYYCTQSYFRRTFGGGTKVEIK | | | | |
| | LCDR1 | | | | |
| | Sequence | SEQ ID | LCDR2 Sequence | SEQ ID | LCDR3 Sequence |
| SEQ ID 39 | KSSQSLLNARTGK | 40 | WASTRES | 49 | TQSYFRRT |
| | NYLA | | | | |

WO2015095392
VH

| SEQ ID 50 | EVQLVESGGGLVQPKGSLKLSCAASGFTFNTYAMNWVRQAPGKGLEWVARIRSKYNNYATYYADSVKDRFTISR | | | | |
|---|---|---|---|---|---|
| | DDSQSILYLQMNNLKTEDTAMYYCVRHGNFGNSYVSWFAYWGQGTLVTVSS | | | | |
| | HCDR1 | | | | |
| | Sequence | SEQ ID | HCDR2 Sequence | SEQ ID | HCDR3 Sequence |
| SEQ ID 25 | TYAMN | 26 | RIRSKYNNYATYYADSVKD | 51 | HGNFGNSYVSWFAY |

VL

| SEQ ID 52 | QAVVTQESALTTSPGETVILTCRSSTGAVTTSNYANWVQEKPDHLFTGLIGGTNKRAPGVPARFSGSLIGDKAA | | | | |
|---|---|---|---|---|---|
| | LTITGAQTEDEATYFCALWYSNLWVFGGGTKLTVL | | | | |
| | LCDR1 | | | | |
| | Sequence | SEQ ID | LCDR2 Sequence | SEQ ID | LCDR3 Sequence |
| SEQ ID 53 | RSSTGAVTTSNYA | 54 | GTNKRAP | 55 | ALWYSNLWV |
| | N | | | | |

TABLE 8
Anti-CD38 Sequences
Clone A
VH

| SEQ ID 56 | QVQLQQSGAELVKPGASVKISCKASGYTFSNYWIEWVKQRPGLGLEWIGDILPGSGTTNYNEKFKGKAT | | | | |
|---|---|---|---|---|---|
| | FTADTSSNTAYMQLSSLISEDSAVYYCAGYGNYLWFTYWGQGTLVTVSA | | | | |
| | HCDR1 Sequence | SEQ ID | HCDR2 Sequence | SEQ ID | HCDR3 Sequence |
| SEQ ID 57 | GYTFSNYWIE | 58 | DILPGSGTTNYNEKFKG | 59 | YGNYLWFTY |

VL

| SEQ ID 60 | DIQMNQSPSSLSASLGDTITITCHASQNINVWLSWYQQKPGNIPKLLIYKASNLHTGVPSRFSGSGSGT | | | | |
|---|---|---|---|---|---|
| | GFTLTISSLQPEDIATYYCQQGQSYPYTFGGGTKLEIK | | | | |
| | LCDR1 Sequence | SEQ ID | LCDR2 Sequence | SEQ ID | LCDR3 Sequence |
| SEQ ID 61 | HASQNINVWLS | 62 | KASNLHT | 63 | QQGQSYPYT |

Clone B
VH

| SEQ ID 64 | QAYLQQSGAEVVRSGASVKMSCKAAGYTFTSYNMHWVKQTPGQGLEWIGYIYPGNGGTNYNQKFKGKAT | | | | |
|---|---|---|---|---|---|
| | LTADTSSTTAYMQISSLISEDSAVYFCARGASMITTGAWFAYWGQGTLVTVSA | | | | |
| | HCDR1 Sequence | SEQ ID | HCDR2 Sequence | SEQ ID | HCDR3 Sequence |
| SEQ ID 65 | GYTFTSYNMH | 66 | YIYPGNGGTNYNQKFKG | 67 | GASMITTGAWFAY |

VL

| SEQ ID 68 | NIVLTQSPASLAVSLGQRATISCRASESLDTYGNSFMYWYQQKPGQPPKLLIYLASSLESGVPARFSGS | | | | |
|---|---|---|---|---|---|
| | GSRTDFTLTIDPVEADDAATYYCHQNHEDPWTFGGGTKLEIK | | | | |
| | LCDR1 Sequence | SEQ ID | LCDR2 Sequence | SEQ ID | LCDR3 Sequence |
| SEQ ID 69 | RASESLDTYGNSFMY | 70 | LASSLES | 71 | HQNHEDPWT |

Clone C
VH

| SEQ ID 72 | EVQLQQSGTVLARPGASVKMSCKASGYTFTRYWMHWVKQRPGQDLEWIGGFYPGNSDTRYNQKFKDKAK | | | | |
|---|---|---|---|---|---|
| | LTAVTSTSTAYMELSSLTNEDSAVYYCTRGDRHDEGFAYWGQGTLVTVSA | | | | |
| | HCDR1 Sequence | SEQ ID | HCDR2 Sequence | SEQ ID | HCDR3 Sequence |
| SEQ ID 73 | GYTFTRYWMH | 74 | GFYPGNSDTRYNQKFKD | 75 | GDRHDEGFAY |

VL

| SEQ ID 76 | DIQMTQSPASLSASVGETVTITCRASGNIHNYLAWYQQKQGKSPQLLVYNAKTLPDGVPSRFSGSGSGT | | | | |
|---|---|---|---|---|---|
| | QYSLKINSLQPEDFGTYYCQNIWTNYTFGGGTKLEIK | | | | |
| | LCDR1 Sequence | SEQ ID | LCDR2 Sequence | SEQ ID | LCDR3 Sequence |
| SEQ ID 77 | RASGNIHNYLA | 78 | NAKTLPD | 79 | QNIWTNYT |

Clone D
VH

| SEQ ID 80 | QIQLVQSGPELKKPGETVKISCKASGYTFTNYGMNWVKQAPGEDLKWMGWLNTNTGEPTYAEEFKGRFA | | | | |
|---|---|---|---|---|---|
| | FSLETSASTAYLQINNLRNEDTATYFCARWYYGSSDNSYAMDYWGQGTSVTVSS | | | | |
| | HCDR1 Sequence | SEQ ID | HCDR2 Sequence | SEQ ID | HCDR3 Sequence |
| SEQ ID 81 | GYTFTNYGMN | 82 | WLNTNTGEPTYAEEFKG | 83 | WYYGSSDNSYAMDY |

VL

| SEQ ID 84 | ETIVTQFPASLSMAIGEKVTIRCITSTDIDDDMNWYQQRPGEPPKLLISEGNTLRPGVPSRFSSSGYGT | | | | |
|---|---|---|---|---|---|
| | DFVFTFENMLSEDVADYYCLQSDSLPYTVGGGTRLEIK | | | | |
| | LCDR1 Sequence | SEQ ID | LCDR2 Sequence | SEQ ID | LCDR3 Sequence |
| SEQ ID 85 | ITSTDIDDDMN | 86 | EGNTLRP | 87 | LQSDSLPYT |

Clone E
VH

| SEQ ID 88 | QVQLQQSGAELVRPGTSVKMSCKAAGYTFTNHWIEWVKQRPGHGLEWIGDIFPGGGYTDYNEKFKGKAT | | | | |
|---|---|---|---|---|---|
| | LTADTSSSTAYMQLSSLTSEDSAIYYCARGRDLAMDYWGQGTSVTVSS | | | | |
| | HCDR1 Sequence | SEQ ID | HCDR2 Sequence | SEQ ID | HCDR3 Sequence |
| SEQ ID 89 | GYTFTNHWIE | 90 | DIFPGGGYTDYNEKFKG | 91 | GRDLAMDY |

VL

| SEQ ID 92 | DIQMNQSPSSLSASLGDTITITCHASQNINVWLGWYQQKPGNIPKLLIYKASNLHTGVPSRFSGSGSGT | | | | |
|---|---|---|---|---|---|
| | GFTLTISSLQPEDIATYYCQQGQSYPYTFGGGTKLEIK | | | | |
| | LCDR1 Sequence | SEQ ID | LCDR2 Sequence | SEQ ID | LCDR3 Sequence |
| SEQ ID 93 | HASQNINVWLG | 94 | KASNLHT | 95 | QQGQSYPYT |

Clone F
VH

| SEQ ID 96 | EVQLQQSGAEFVRPGASVKLSCTASGFNIKDSLMHWVKQRPEQGLEWIGWIDPEDDKTKYAPKFQDEAT | | | | |
|---|---|---|---|---|---|
| | LTADTSSNTAYLHLSSLTSEDTAIYYCARRFLRGFAYWGQGTLVTVSA | | | | |
| | HCDR1 Sequence | SEQ ID | HCDR2 Sequence | SEQ ID | HCDR3 Sequence |
| SEQ ID 97 | GFNIKDSLMH | 98 | WIDPEDDKTKYAPKFQD | 99 | RFLRGFAY |

VL

| SEQ ID 100 | DIVMTQSPSSLSVSAGEKVTMSCKSSQSLLNSGNQKNYLAWYQQKPGQPPKLLIYGASTRESGVPDRFT | | | | |
|---|---|---|---|---|---|
| | GSGSGTDFTLTISSVQAEDLAVYYCQNDHSYPYTFGGGTKLEIK | | | | |
| | LCDR1 Sequence | SEQ ID | LCDR2 Sequence | SEQ ID | LCDR3 Sequence |
| SEQ ID 101 | KSSQSLLNSGNQKNYLA | 102 | GASTRES | 103 | QNDHSYPYT |

Clone G
VH

| SEQ ID 104 | QVQLQQPGPELVKPVASVKISCKTSDYAFSSSWMNWVKQRPGKGLEWIGWIYPGDGDTNYNGKFKGKAT | | | | |
|---|---|---|---|---|---|
| | LTADKSSSTAYMQLSSLTSEDSAVYFCARGRWGMDNWGQGTSVTVSS | | | | |
| | HCDR1 Sequence | SEQ ID | HCDR2 Sequence | SEQ ID | HCDR3 Sequence |
| SEQ ID 105 | DYAFSSSWMN | 106 | WIYPGDGDTNYNGKFKG | 107 | GRWGMDN |

VL

| SEQ ID 108 | DIVMTQSHKFMSTSVGDRVSITCKASQNVVTAVAWYQQKPGQSPKLLIYWASSRHTGVPDRFTGSGSGT | | | | |
|---|---|---|---|---|---|
| | DFTLTISNVQSEDLADYFCQQYSNYPFTFGSGTKLEIK | | | | |
| | LCDR1 Sequence | SEQ ID | LCDR2 Sequence | SEQ ID | LCDR3 Sequence |
| SEQ ID 109 | KASQNVVTAVA | 110 | WASSRHT | 111 | QQYSNYPFT |

Clone H
VH

| SEQ ID 112 | QVPLHQSGPELIKPGASLKISCKASDSAFNNSWMNWVRQRPGKGLEWIGWIYPGDDDTKYNGKFMGKAT | | | | |
|---|---|---|---|---|---|
| | LTADKSSGTAYMQLSSLTSEDSAVYFCARGRWGMDYWGQGTSVTVSS | | | | |
| | HCDR1 Sequence | SEQ ID | HCDR2 Sequence | SEQ ID | HCDR3 Sequence |
| SEQ ID 113 | DSAFNNSWMN | 114 | WIYPGDDDTKYNGKFMG | 115 | GRWGMDY |

VL

| SEQ ID 116 | DIVMTQFHNFMSTSVGDRVNINCKASQNVVTAVAWYQQKPGQSPKLLIYWASTRHTGVPDRFTGSGSGT | | | | |
|---|---|---|---|---|---|
| | DFTLTISNVQSEDLADYFCQQYSNYPFTFGSGTKLEIK | | | | |
| | LCDR1 Sequence | SEQ ID | LCDR2 Sequence | SEQ ID | LCDR3 Sequence |
| SEQ ID 117 | KASQNVVTAVA | 118 | WASTRHT | 119 | QQYSNYPFT |

Clone I
VH

| SEQ ID 120 | QVPLQQSGPELVKPGASVKISCKASDSAFNNSWTIWVRQRPGKGLEWIGWIYPGDDDTKYNGKFMGKAT | | | | |
|---|---|---|---|---|---|
| | LTADKSSGTAYMQLSSLTSEDSAVYFCARGRWGMDYWGQGTSVTVSS | | | | |
| | HCDR1 Sequence | SEQ ID | HCDR2 Sequence | SEQ ID | HCDR3 Sequence |
| SEQ ID 121 | DSAFNNSWTI | 122 | WIYPGDDDTKYNGKFMG | 123 | GRWGMDY |

VL

| SEQ ID 124 | DIVMTQSRKFMSTSVGDRVSISCKASQNVVTAVAWYQQKPGQSPKLLIYWASTRHTGVPDRFTGSGSGT | | | | |
|---|---|---|---|---|---|
| | DFTLTISNVQSEDLADYFCQQYSNYPFTFGSGTELEIK | | | | |
| | LCDR1 Sequence | SEQ ID | LCDR2 Sequence | SEQ ID | LCDR3 Sequence |
| SEQ ID 125 | KASQNVVTAVA | 126 | WASTRHT | 127 | QQYSNYPFT |

Humanized Clone B
BVH1

| SEQ ID 128 | QVQLVQSGAEVKKPGSSVKVSCKASGYTFTSYNMHWVRQAPGQGLEWMGYIYPGNGGTNYNQKFKGRVT | | | | |
|---|---|---|---|---|---|
| | ITADESTSTAYMELSSLRSEDTAVYYCARGASMITTGAWFAYWGQGTLVTVSS | | | | |
| | HCDR1 Sequence | SEQ ID | HCDR2 Sequence | SEQ ID | HCDR3 Sequence |
| SEQ ID 65 | GYTFTSYNMH | 66 | YIYPGNGGTNYNQKFKG | 67 | GASMITTGAWFAY |

BVH2

| SEQ ID 129 | QVQLVQSGAEVKKPGSSVKVSCKASGYTFTSYNMHWVRQAPGQGLEWMGYIYPGNGGTNYNQKFKGRVT | | | | |
|---|---|---|---|---|---|
| | ITADTSTSTAYMELSSLRSEDTAVYYCARGASMITTGAWFAYWGQGTLVTVSS | | | | |
| | HCDR1 Sequence | SEQ ID | HCDR2 Sequence | SEQ ID | HCDR3 Sequence |
| SEQ ID 65 | GYTFTSYNMH | 66 | YIYPGNGGTNYNQKFKG | 67 | GASMITTGAWFAY |

BVH3

| SEQ ID 130 | QVQLVQSGAEVKKPGSSVKVSCKASGYTFTSYNMHWVRQAPGQGLEWMGYIYPGSGGTNYNQKFKGRVT | | | | |
|---|---|---|---|---|---|
| | ITADESTSTAYMELSSLRSEDTAVYYCARGASMITTGAWFAYWGQGTLVTVSS | | | | |
| | HCDR1 Sequence | SEQ ID | HCDR2 Sequence | SEQ ID | HCDR3 Sequence |
| SEQ ID 65 | GYTFTSYNMH | 134 | YIYPGSGGTNYNQKFKG | 67 | GASMITTGAWFAY |

BVH4

| SEQ ID 131 | QVQLVQSGAEVKKPGSSVKVSCKASGYTFTSYNMHWVRQAPGQGLEWMGYIYPGNGGTSYNQKFKGRVT | | | | |
|---|---|---|---|---|---|
| | ITADESTSTAYMELSSLRSEDTAVYYCARGASMITTGAWFAYWGQGTLVTVSS | | | | |
| | HCDR1 Sequence | SEQ ID | HCDR2 Sequence | SEQ ID | HCDR3 Sequence |
| SEQ ID 65 | GYTFTSYNMH | 201 | YIYPGNGGTSYNQKFKG | 67 | GASMITTGAWFAY |

BVH5

| SEQ ID 132 | QVQLVQSGAEVKKPGSSVKVSCKASGYTFTSYNMHWVRQAPGQGLEWMGYIYPGSGGTSYNQKFKGRVT | | | | |
|---|---|---|---|---|---|
| | ITADESTSTAYMELSSLRSEDTAVYYCARGASMITTGAWFAYWGQGTLVTVSS | | | | |
| | HCDR1 Sequence | SEQ ID | HCDR2 Sequence | SEQ ID | HCDR3 Sequence |
| SEQ ID 65 | GYTFTSYNMH | 135 | YIYPGSGGTSYNQKFKG | 67 | GASMITTGAWFAY |

BVL1

| SEQ ID 133 | DIQMTQSPSSVSASVGDRVTITCRASESLDTYGNSFMYWYQQKPGKAPKLLIYLASSLESGVPSRFSGS | | | | |
|---|---|---|---|---|---|
| | GSGTDFTLTISSLQPEDFATYYCHQNHEDPWTFGQGTKVEIK | | | | |
| | LCDR1 Sequence | SEQ ID | LCDR2 Sequence | SEQ ID | LCDR3 Sequence |
| SEQ ID 69 | RASESLDTYGNSFMY | 70 | LASSLES | 71 | HQNHEDPWT |

Humanized Clone E
EVH1

| SEQ ID 136 | QVQLVQSGAEVKKPGSSVKVSCKASGYTFTNHWIEWVRQAPGQGLEWMGDIFPGGGYTDYNEKFKGRVT | | | | |
|---|---|---|---|---|---|
| | ITADESTSTAYMELSSLRSEDTAVYYCARGRDLAMDYWGQGTLVTVSS | | | | |
| | HCDR1 Sequence | SEQ ID | HCDR2 Sequence | SEQ ID | HCDR3 Sequence |
| SEQ ID 89 | GYTFTNHWIE | 90 | DIFPGGGYTDYNEKFKG | 91 | GRDLAMDY |

EVH2

| SEQ ID 137 | QVQLVQSGAEVKKPGSSVKVSCKASGYTFTNHWIEWVRQAPGQGLEWIGDIFPGGGYTDYNEKFKGRAT | | | | |
|---|---|---|---|---|---|
| | ITADESTSTAYMELSSLRSEDTAVYYCARGRDLAMDYWGQGTLVTVSS | | | | |
| | HCDR1 Sequence | SEQ ID | HCDR2 Sequence | SEQ ID | HCDR3 Sequence |
| SEQ ID 89 | GYTFTNHWIE | 90 | DIFPGGGYTDYNEKFKG | 91 | GRDLAMDY |

EVL1

| SEQ ID 138 | DIQMTQSPSSLSASVGDRVTITCHASQNINVWLGWYQQKPGKAPKLLIYKASNLHTGVPSRFSGSGSGT | | | | |
|---|---|---|---|---|---|
| | DFTLTISSLQPEDFATYYCQQGQSYPYTFGQGTKVEIK | | | | |
| | LCDR1 Sequence | SEQ ID | LCDR2 Sequence | SEQ ID | LCDR3 Sequence |
| SEQ ID 93 | HASQNINVWLG | 94 | KASNLHT | 95 | QQGQSYPYT |

EVL2

| SEQ ID 139 | DIQMTQSPSSLSASVGDRVTITCRASQNINVWLGWYQQKPGKAPKLLIYKASNLHTGVPSRFSGSGSGT | | | | |
|---|---|---|---|---|---|
| | DFTLTISSLQPEDFATYYCQQGQSYPYTFGQGTKVEIK | | | | |
| | LCDR1 Sequence | SEQ ID | LCDR2 Sequence | SEQ ID | LCDR3 Sequence |
| SEQ ID 143 | RASQNINVWLG | 94 | KASNLHT | 95 | QQGQSYPYT |

EVL3

| SEQ ID 140 | DIQMTQSPSSLSASVGDRVTITCSASQNINVWLGWYQQKPGKAPKLLIYKASNLHTGVPSRFSGSGSGT | | | | |
|---|---|---|---|---|---|
| | DFTLTISSLQPEDFATYYCQQGQSYPYTFGQGTKVEIK | | | | |
| | LCDR1 Sequence | SEQ ID | LCDR2 Sequence | SEQ ID | LCDR3 Sequence |
| SEQ ID 144 | SASQNINVWLG | 94 | KASNLHT | 95 | QQGQSYPYT |

EVL4

| SEQ ID 141 | DIQMTQSPSSLSASVGDRVTITCGASQNINVWLGWYQQKPGKAPKLLIYKASNLHIGVPSRFSGSGSGT | | | | |
|---|---|---|---|---|---|
| | DFTLTISSLQPEDFATYYCQQGQSYPYTFGQGTKVEIK | | | | |
| | LCDR1 Sequence | SEQ ID | LCDR2 Sequence | SEQ ID | LCDR3 Sequence |
| SEQ ID 145 | GASQNINVWLG | 94 | KASNLHT | 95 | QQGQSYPYT |

EVL5

| SEQ ID 142 | DIQMTQSPSSLSASVGDRVTITCEASQNINVWLGWYQQKPGKAPKLLIYKASNLHTGVPSRFSGSGSGT | | | | |
|---|---|---|---|---|---|
| | DFTLTISSLQPEDFATYYCQQGQSYPYTFGQGTKVEIK | | | | |
| | LCDR1 Sequence | SEQ ID | LCDR2 Sequence | SEQ ID | LCDR3 Sequence |
| SEQ ID 146 | EASQNINVWLG | 94 | KASNLHT | 95 | QQGQSYPYT |

Humanized OKT10
OKT10VH1

| | |
|---|---|
| SEQ ID | QITLKESGPTLVKPTQTLTLTCTASGFDFSRSWMNWIRQPPGKALEWLAEINPDSSTINYTTSLKDRFT |
| 147 | ISRDTSKNQVVLTMTNMDPVDTATYYCARYGNWFPYWGQGTLVTVSS |

OKT10VH2

| | |
|---|---|
| SEQ ID | EVQLLESGGGLVQPGGSLRLSCAASGFDFSRSWMNWVRQAPGKGLEWVSEINPDSSTINYATSLKDRFT |
| 148 | ISRDNSKNTLYLQMNSLRAEDTAVYYCARYGNWFPYWGQGTLVTVSS |

OKT10VH3

| | |
|---|---|
| SEQ ID | QLQLQESGPGLVKPSETLSLTCTASGFDFSRSWMNWIRQPPGKGLEWIGEINPDSSTINYATSLKDRFT |
| 149 | ISRDTSKNQLSLKLSSVTAADTAVYYCARYGNWFPYWGQGTLVTVSS |

OKT10VL1

| | |
|---|---|
| SEQ ID | AIQLTQSPSSLSASVGDRVTITCKASQNVDTNVAWYQQKPGKAPKALIYSASYRYSGVPSRFSGSGSGT |
| 150 | DFTLTISSLQPEDFATYYCQQYDSYPLTFGQGTKVEIK |

OKT10VL2

| SEQ ID | DIQMTQSPSSLSASVGDRVTITCKASQNVDTNVAWYQQKPGKAPKALIYSASYRYSGVPSRFSGSGSGT |
|---|---|
| 151 | DFTLTISSLQPEDFATYYCQQYDSYPLTFGQGTKVEIK |
| | OKT10VL3 |
| SEQ ID | DIQMTQSPSSLSASVGDRVTITCRASQNVDTNVAWYQQKPGKAPKALIYSASYRYSGVPSRFSGSGSGT |
| 152 | DFTLTISSLQPEDFATYYCQQYDSYPLTFGQGTKVEIK |

OKT10VL4

| | |
|---|---|
| SEQ ID | DIQMTQSPSSLSASVGDRVTITCSASQNVDTNVAWYQQKPGKAPKALIYSASYRYSGVPSRFSGSGSGT |
| 153 | DFTLTISSLQPEDFATYYCQQYDSYPLTFGQGTKVEIK |

OKT10VL5

| | |
|---|---|
| SEQ ID | DIQMTQSPSSLSASVGDRVTITCGASQNVDTNVAWYQQKPGKAPKALIYSASYRYSGVPSRFSGSGSGT |
| 154 | DFTLTISSLQPEDFATYYCQQYDSYPLTFGQGTKVEIK |

OKT10VL6

| | |
|---|---|
| SEQ ID | DIQMTQSPSSLSASVGDRVTITCEASQNVDINVAWYQQKPGKAPKALIYSASYRYSGVPSRFSGSGSGT |
| 155 | DFTLTISSLQPEDFATYYCQQYDSYPLTFGQGTKVEIK |

Humanized HB7
HB7VH1

| | |
|---|---|
| SEQ ID | EVQLVESGGGLVQPGGSLRLSCAVSGFSLISYGVHWVRQAPGKGLEWLGVIWRGGSTDYNAAFMSRLTI |
| 156 | SKDNSKNTVYLQMNSLRAEDTAVYYCAKTLITTGYAMDYWGQGTLVTVSS |

HB7VH2

| | |
|---|---|
| SEQ ID | QVQLQESGPGLVKPSETLSLTCTVSGFSLISYGVHWVRQPPGKGLEWLGVIWRGGSTDYNAAFMSRLTI |
| 157 | SKDTSKNQVSLKLSSVTAADTAVYYCAKTLITTGYAMDYWGQGTLVTVSS |

HB7VH3

| | |
|---|---|
| SEQ ID | QVQLQESGPGLVKPSDTLSLTCAVSGFSLISYGVHWVRQPPGKGLEWLGVIWRGGSTDYNAAFMSRLTM |
| 158 | SKDTSKNQVSLKLSSVTAVDTAVYYCAKTLITTGYAMDYWGQGTLVTVSS |

HB7VL1

| | |
|---|---|
| SEQ ID | DIQMTQSPSSLSASVGDRVTITCKASEDIYNRLAWYQQKPGKAPKLLISGATSLETGVPSRFSGSGSGT |
| 159 | DYTLTISSLQPEDFATYYCQQYWSTPTFGQGTKVEIK |

HB7VL2

| | |
|---|---|
| SEQ ID | DIQMTQSPSSLSASVGDRVTITCKASEDIYNRLAWYQQKPGKAPKLLISGATSLETGVPSRFSGSGSGT |
| 160 | DYTFTISSLQPEDIATYYCQQYWSTPTFGQGTKVEIK |

HB7VL3

| | |
|---|---|
| SEQ ID | EIVLTQSPDFQSVTPKEKVTITCKASEDIYNRLAWYQQKPDQSPKLLISGATSLETGVPSRFSGSGSGT |
| 161 | DYTLTINSLEAEDAATYYCQQYWSTPTFGQGTKVEIK |

What is claimed is:

1. A method of treating an autoimmune disorder comprising administering to a subject in need of treatment an effective amount of a multimeric bispecific anti-CD38/anti-CD3 antibody that comprises five bivalent binding units and a modified J chain, wherein each binding unit comprises two IgM heavy chains, each comprising the amino acid sequence SEQ ID NO: 198, and two light chains, each comprising the amino acid sequence SEQ ID NO: 199, and wherein the modified J chain comprises the amino acid sequence SEQ ID NO: 175.

2. The method of claim 1, wherein the autoimmune disorder is one in which CD38-expressing cells contribute to chronic inflammation in the subject.

3. The method of claim 2, wherein the autoimmune disorder is systemic lupus erythematosus (SLE), antiphospholipid syndrome (APS), idiopathic thrombocytopenia purpura (ITP), warm autoimmune hemolytic anemia (wAIHA), multiple sclerosis (MS), myasthenia gravis (MG), pemphigus vulgaris (PV), anti-neutrophil cytoplasmic autoantibody (ANCA) associated vasculitis (AAV), thyroid eye disease (TED), membranous glomerulonephritis (MGN), Neuromyelitis optica (NMO), Guillain-Barré syndrome (GBS), chronic inflammatory demyelinating polyradiculoneuropathy (CIDP), IgA nephropathy, Goodpasture's syndrome, granulomatosis with polyangiitis (GPA), microscopic polyangiitis (MPA), Sjögren syndrome, Behcet's disease, alopecia areata, immunoglobulin G4-related disease (IgG4-RD), phospholipase A2 receptor-associated membranous nephropathy (PLA2R MN), myositis, type 1 diabetes, systemic sclerosis, or rheumatoid arthritis (RA).

4. The method of any one of claims 1 to 3, wherein the autoimmune disorder is SLE.

5. The method of claim 4, wherein the SLE comprises lupus nephritis (LN).

6. The method of any one of claims 1 to 3, wherein the autoimmune disorder is RA.

7. The method of any one of claims 1 to 3, wherein the autoimmune disorder is MG.

8. The method of any one of claims 1 to 7, wherein prior to the administering, the subject had previously been treated with a biologic autoimmune disorder therapy.

9. The method of claim 8, wherein the autoimmune disorder is SLE, and prior to the administering, the subject had previously been treated with belimumab.

10. The method of claim 8, wherein the autoimmune disorder is MS, and prior to the administering, the subject had previously been treated with rituximab.

11. The method of claim 8 wherein the autoimmune disorder is RA, and prior to the administering, the subject had previously been treated with infliximab, adalimumab, or etanercept.

12. The method of any one of claims 1 to 11, wherein the administration of the multimeric bispecific anti-CD38/anti-CD3 antibody is via intravenous infusion.

13. The method of any one of claims 1 to 11, wherein the administration of the multimeric bispecific anti-CD38/anti-CD3 antibody is via subcutaneous injection.

14. The method of any one of claims 1 to 13, wherein the subject is a human.
